SEQUENCE LISTING (2 sequences)

SEQ ID NO: 1 - DNA, 24 bp, Artificial Sequence
  custom primer
tgaaacgcttgctgcaacgccaaa

SEQ ID NO: 2 - DNA, 30 bp, Artificial Sequence
  custom primer
aaagcaattggcggtgatgtaaacactatg

(19) United States
(12) Patent Application Publication
(10) Pub. No.: US 2012/0071643 A1
(43) Pub. Date: Mar. 22, 2012

(54) SYSTEM AND METHODS FOR PURIFYING BIOLOGICAL MATERIALS

(75) Inventors: Jeffrey L. Helfer, Victor, NY (US); Rhiannon R. Gaborski, Rochester, NY (US); Maurice A. Kildal, Webster, NY (US)

(73) Assignee: DIFFINITY GENOMICS, INC., West Henrietta, NY (US)

(21) Appl. No.: 13/201,415

(22) PCT Filed: Feb. 13, 2010
(86) PCT No.: PCT/US2010/024201

§ 371 Date: Nov. 23, 2011

Related U.S. Application Data
(60) Provisional application No. 61/152,680, filed on Feb. 14, 2009.
(60) Provisional application No. 61/290,333, filed on Dec. 28, 2009.

(51) Int. Cl. C07H 21/00; B01D 43/00; B82Y 99/00

(52) U.S. Cl. 536/25.4; 422/261; 977/773

(57) ABSTRACT

Fluid sample purification systems and methods are provided for isolating molecules of interest in a fluid sample. The fluid sample purification system has a housing with a distal end and distal opening adapted for the passage of a fluid and a proximal end and proximal opening adapted for passage of a fluid. A distal retainer is located inside the housing and above the distal opening. A proximal retainer is located inside the housing between the distal retainer and the proximal opening, or is located adjacent to, in contact with, or over the proximal opening. The system also comprises adsorption material, e.g., functionalized particles, inside the housing and confined between the distal retainer and the proximal retainer. The adsorption material adsorbs undesirable material while simultaneously rejecting desirable materials. Methods are also provided for isolating molecules of interest using the fluid sample purification system.

### CROSS-REFERENCE TO RELATED APPLICATIONS

This application claims priority to and the benefit of co-pending U.S. provisional patent application Ser. No. 61/152,680, filed Feb. 14, 2009, entitled “Devices for Purifying Biological Materials” and Ser. No. 61/290,333 entitled “System and Methods for Purifying Biological Materials,” filed Dec. 28, 2009, both by Jeffrey L. Helfer and Rhiannon R. Gaborski, and each of which is incorporated herein by reference in its entirety.

### STATEMENT REGARDING FEDERALLY SPONSORED RESEARCH OR DEVELOPMENT

The disclosed invention was made with government support under STTR grant numbers R41RR024968-01 and 2R42RR024968-02 from the National Institutes of Health. The government has certain rights in this invention.

### 1. TECHNICAL FIELD

The invention relates to systems and methods for purifying fluids. The invention also relates to systems and methods for separating molecules of interest from undesirable material.

### 2. BACKGROUND OF THE INVENTION

Differences in individual nucleic acid (“NA”) sequences, such as DNA sequences are known to determine the presence of disease, predisposition to future medical conditions, and likely response to specific therapeutic treatments. Consequently, the analysis of NA is growing rapidly. For example, DNA sequence analysis and other types of DNA analysis are typically done following the use of DNA amplification methods, most commonly polymerase chain reaction (“PCR”), that are used to increase the amount of a desired genetic sequence or “target” in a sample. Purification of a particular DNA target from the PCR amplification mixture is necessary, for example, for effective DNA sequencing, since failure to remove impurities, such as the excess primers and nucleotides (“dNTPs”) introduced during the DNA amplification process, can interfere with sequencing chemistry and can lead to inaccurate results. Purification of PCR amplified DNA is required in many other applications, such as microarray analysis and cloning.

### 2.1 Purification of Nucleic Acids

Existing products for nucleic acid purification typically use two different purification technologies, enzymatic digestion and total nucleic acid (e.g., DNA) adsorption, each of which has serious shortcomings. Enzymatic digestion methods involve the addition of enzymes to chemically degrade impurities. The method uses a multi-step protocol that requires incubating samples at two temperatures for approximately 30 minutes or more to produce samples suitable for analysis. However, since enzymatic methods do not remove impurities, but rather, render them inactive, the presence of inactivated impurities in solution interferes with subsequent methods for nucleic acid quantification, i.e., the impurities still affect the UV absorbance methods that are often used to quantify nucleic acid target concentration. As a result, quantification of target nucleic acids (e.g., DNA, RNA, plasmids, etc.) purified by enzymatic methods is limited to techniques such as gel electrophoresis, which is semi-quantitative at best.

Existing total adsorption methods begin by retaining impurities as well as target DNA onto adsorbing surfaces, such as membranes or magnetic beads, requiring multiple, complex, time-consuming and expensive chemical-mechanical processes to purify and release the desired target nucleic acid. The latter purification methods are often referred to as bind-wash-elute methods.

Existing nucleic acid purification products are often criticized by users for leaving too many impurities in the product and thus resulting in poorer test results; high cost (both purification kit purchase price and end-user labor); and for being slow and requiring substantial “hands-on” time (10-20 minutes or more per sample). Total adsorption methods, in particular, require multiple steps to, e.g., immobilize all nucleic acid amplification reaction components and to selectively elute (extract) the desired nucleic acid target. The labor intensive nature of these products drives actual purification costs well above the $1-$2 (USD) disposable costs currently required to clean up a single nucleic acid sample, and provides many opportunities where operator error can adversely affect the purification process. Use of many of the existing nucleic acid purification products also involves the addition of solvents that can inhibit nucleic acid analysis if the solvents are not effectively removed.

Recovering a high percentage of a fluid sample from a sample purification process is also very important, particularly when small volumes of sample are available. Many existing sample purification processes often retain a high percentage of the fluid sample, owing in part to the use of multiple purification process steps, each of which can lose a small portion of the sample. Maintaining a high degree of fluid sample recovery is an important requirement of any sample purification process.

Existing purification products are generally sold as “kits”, which typically contain the manufacturers' materials, such as spin columns with adsorption membranes and special reagents. The end-user typically provides fluid handling consumables (e.g., pipette tips) and commonly used reagents.

### 2.2 Functional Pipette Tips

Chemical reactions and analysis involving fluids often use pipette tips to manipulate the fluids during the course of the reaction or analysis. To help improve the efficiency and reduce the cost of related workflows, manufacturers often integrate elements of the reaction or analysis into the pipette tip itself—what are typically referred to a “functional” pipette tips. These chemical reactions and analysis often include the use of specially treated particles within the pipette tip to improve the interaction between the sample and the chemistry provided by the particles.

Confining the particles with distally and proximally placed particle retainers often creates pipette tip performance problems. One such problem is a reduction in fluid aspirate and dispense times, due to the flow resistance created by the filters. Another problem is the propensity of the retainer to retain sample fluid, thereby reducing sample yield. Yet another problem is the variability in pore size within a filter, whether the filter is comprised of a ceramic or polymer frit or fragile membrane. Still another problem with existing filter designs is inconsistent fluid flow resistance across filters in multiple pipette tips. Still another problem with existing filter designs is high cost.

There are currently several functional pipette tips available commercially. One example is Thermo Fisher Scientifics Aspire™ pipette tip (similar to the tip disclosed in WO88/09201 entitled “Process and Device for Separating and Cleaning Molecules” by Colpan et al.). This design has considerable performance shortcomings. One shortcoming is long sample processing time. Underlying causes include low fluid flow rate due to the use of a high resistance filter as well as the use of a second filter to contain the adsorption media prior to and during use, hydrophobic adsorption media which require pre-wetting, entrapment of air with the reaction chamber, and clogging of the filters by the relatively large volume of adsorption media required

Another shortcoming is poor reliability. Underlying factors include the potential to entrap air within the reaction chamber.

Another shortcoming is inherently higher product manufacturing cost.

Underlying factors include the use of a second filter and relatively large volume of adsorption media required, which also increases retained sample fluid and increases pipette tip manufacturing cost.

Another example of a functional pipette tip is Millipore's Zip-Tip™ (similar tips known in the art, see, e.g., tip disclosed in U.S. Pat. No. 6,048,457 entitled Cast Membrane Structures for Sample Preparation by Kopaciewicz et al.). This design also has considerable performance shortcomings, which include long sample processing time. Underlying causes include high fluid flow resistance caused by the porous matrix used to contain adsorption material.

Another shortcoming is limited adsorption surface area. Underlying causes include the limited number of adsorption materials (e.g., particles) that can be immobilized within the porous matrix used, as well as the reduction in adsorption material surface area that occurs when adsorption materials are bound to a surface (e.g., reduction in exposed particle surface area).

Another shortcoming is potential for poor yield of target materials. The large surface area of the porous matrix, relative to for example, the surface area of adsorption particles immobilized within the matrix, can interact with samples and remove target materials thereby reducing target yield.

Yet another shortcoming is the large amount of sample fluid that can remain in the porous matrix used to contain the adsorption material.

There is therefore a need in the art for a fluid sample purification system that has high target yield, high reliability, rapid sample processing time, extensive adsorptive capacity, high sample volume recovery and low manufacturing cost.

Citation or identification of any reference in Section 2, or in any other section of this application, shall not be considered an admission that such reference is available as prior art to the invention.

### 3. SUMMARY OF THE INVENTION

A fluid sample purification system and method is provided that is rapid, user-friendly and entails no addition or subsequent removal of analytes, reagents, or reaction products. The fluid sample purification system and method reduce the potential for error and results in a user-friendly process that takes 1 minute or less and removes over 95% of the impurities (i.e., primers and unincorporated nucleotides) while leaving ample target biomolecules for subsequent analysis. Target biomolecules for analysis can include, but are not limited to single-stranded nucleic acids such as RNA or PCR primers, double-stranded nucleic acids, such as DNA (e.g., genomic DNA, plasmid DNA) or PCR primer-dimers, nucleotides such as ribonucleotides, enzymes such as DNA polymerases, RNA polymerases, labeled probes, restriction enzymes or reverse transcriptases, dyes such as intercalating agents, water, heavy metals, toxins such as blood-borne toxins (e.g., urea or creatinine), blood components including metabolites, electrolytes, hormones or drugs, and antibodies or antigens.

In one embodiment, a fluid sample purification system for isolating a molecule of interest in a fluid sample is provided. The fluid sample purification system can comprise a housing having a distal end with a distal opening adapted for the passage of a fluid and a proximal end with a proximal opening adapted for passage of a fluid; a distal retainer inside the housing and above the distal opening; and adsorption material, e.g., a plurality of functionalized particles.

The fluid sample purification system can also comprise a proximal retainer, wherein the proximal retainer is located inside the housing and between the distal retainer and the proximal opening, or wherein the proximal retainer is adjacent to, in contact with, covering or sealing the proximal opening.

The fluid sample purification system can also comprise a housing and adsorption material, but not comprise a distal or a proximal retainer.

In one embodiment, the housing is pipette tip. In another embodiment, the housing is a column, e.g., a capillary tube, a spin-column or a multi-well microtiter plate. In another embodiment, the system can comprise a plurality of housings.

In another embodiment, the adsorption material is a plurality of functionalized particles, wherein the plurality of functionalized particles is located inside the housing and confined between the distal retainer and the proximal retainer.

In another embodiment, the functionalized particles of the plurality adsorb undesirable material, material that is not an analyte of interest, or material to be separated from the molecule of interest.

In another embodiment, the functionalized particles of the plurality are differentially functionalized particles, i.e., the plurality can comprise two or more types of functionalized particles.

In another embodiment, the adsorption material is applied to or coated on the inside of the housing.

In another embodiment, loosely contained adsorption materials (e.g., particles) are contained within the pipette tip or column. In another embodiment, tightly packed or confined adsorption materials are used. In another embodiment adsorption materials are immobilized on a fluid-contactable surface or in nanopores or other features located in or on the fluid-contactable surface.

In another embodiment, the fluid sample purification system comprises adsorption materials within the housing, wherein the adsorption materials are immobilized on particles or in nanopores or other features located in or on the particles.

In another embodiment, the adsorption material is porous. In a specific embodiment, the functionalized particles are porous, thus providing a larger adsorption surface area with the ability to effectively scavenge a larger mass of impurities while simultaneously small enough to minimize the volume of retained fluid sample while still large enough to minimize filter or retainer clogging. In one embodiment, the porous particles significantly increase (i.e., 10-20×) adsorption capacity.

In another embodiment, the adsorption material or the functionalized particles of the plurality reject (or exclude) the molecule of interest. In another embodiment, undesirable materials in the fluid sample are adsorbed while the molecule of interest or desirable material in the fluid sample is simultaneously repelled or rejected.

In another embodiment, the distal retainer and the proximal retainer are spaced sufficiently far apart to form a void in the presence of the plurality of functionalized particles, and wherein the void is dimensioned so that functionalized particles can travel freely within the void allowing for thorough mixing between the functionalized particles and the fluid sample when the fluid sample is in the void.

In another embodiment, the distal retainer and the proximal retainer are spaced sufficiently close together to confine the plurality of functionalized particles in closely packed configuration so as to form little or no void between the members of the plurality of functionalized particles.

In another embodiment, the system comprises fluid flow conduits associated with the distal retainer. The fluid flow conduits can run axially on or in the inner surface of the housing and an insert. In a specific embodiment, the axially-running fluid flow conduits and the insert form the distal retainer.

In another embodiment, the axially-running fluid flow conduits are formed by the space between the housing and the insert.

In another embodiment, the widths of the flow conduits are chosen to minimize fluid flow resistance.

In another embodiment, the insert is positioned within the housing by insert positioning tabs, thereby creating axially-running fluid flow conduits, wherein the axially-running fluid flow conduits and the insert form the distal retainer.

In another embodiment, the axially-running fluid flow conduits are positioned in the inner wall of the housing, wherein the widths of the flow conduits are chosen to minimize fluid flow resistance.

In another embodiment, the axially-running fluid flow conduits are hemispherical shaped conduits created between the distal end and the distal retainer, and the conduits provide a large flow-through area and low resistance to fluid flow.

In another embodiment, the axially-running fluid flow conduits create less resistance to fluid flow than an equivalent thickness of porous material and thickness of the flow restrictor is minimized.

In another embodiment, the length of each conduit is less than the diameter of the conduit and wherein the length of the conduit is between 10% and 50% of the size of distal retainer.

In another embodiment, the distal retainer has:
a. sufficiently small pore size or fluid flow conduits to retain the plurality of functionalized particles, and
b. sufficiently high fluid sample flow rate at a desired applied pressure to avoid damaging the molecule of interest.

In another embodiment, the adsorption materials within the housing adsorb undesirable materials (e.g., unincorporated nucleic acid amplification primers and nucleotides) while simultaneously rejecting desirable materials (e.g., PCR amplicons) in a single step.

In another embodiment, the fluid sample purification system preferentially adsorbs (or binds) or rejects different types of molecules of interest, such as biomolecules, e.g., single-stranded nucleic acids such as RNA or PCR primers, double-stranded nucleic acids, such as DNA (e.g., genomic DNA, plasmid DNA) or PCR primer-dimers, nucleotides such as ribonucleotides, enzymes such as DNA polymerases, RNA polymerases, labeled probes, restriction enzymes or reverse transcriptases, dyes such as intercalating agents, water, heavy metals, toxins such as blood-borne toxins (e.g., urea or creatinine), blood components including metabolites, electrolytes, hormones or drugs, and antibodies or antigens.

In one embodiment, the fluid sample purification system comprises functionalized particles that are small enough in size to avoid rapid sedimentation in solution, while simultaneously large enough to enable the use of high-flow rate filters and avoid suspension in air (i.e., “dusting”) during product manufacturing.

In another embodiment, the distal or proximal retainer can comprise a material selected from the group consisting of porous polymer or ceramic matrix; perforated polymer, ceramic or metal (e.g., a solid retainer or “plug”); porous polymer, glass or metal fabric; and porous polymer, glass, or metal filaments in the form of a wool.

In another embodiment, the fluid sample purification system can comprise a distal retainer with a flow conduit or flow channel size that effectively retains the optimized particle size described above while simultaneously enabling a high fluid sample flow rate at available applied pressures and avoiding damage to the target material (e.g., ds-DNA).

In one embodiment in which the distal retainer is a filter, the filter pore size is 10-40 μm.

In another embodiment, the fluid sample purification system can comprise a distal retainer (e.g., filter) that is of sufficient size to provide consistent resistance, i.e., low tip-to-tip variation in resistance, to fluid flow at low applied pressures, while simultaneously small enough in size to minimize retained sample volume and by placement nearer the distal end of the housing (e.g., the pipette tip nozzle), minimize sample dead volume within the housing. In one embodiment, the retainer (or filter) has a physical size of 1.0 to 2.0 mm in diameter.

In another embodiment, the fluid sample purification system can comprise a distal retainer that comprises (or is treated with) easily wettable (e.g., hydrophilic) material that enables low fluid intrusion pressure (i.e. minimize the pressure required to initiate the flow of fluid through the retainer) and low fluid extrusion pressure (i.e. minimize the pressure required to force fluid out of the retainer) while not adsorbing target.

In another embodiment, the adsorption material (e.g., porous functionalized particles) is treated to increase wettability.

In another embodiment, the adsorption material is a porous dissolvable material.

In another embodiment, the fluid sample purification system comprises a distal retainer that uses a pore size that enables low fluid intrusion and extrusion pressures while not passing functionalized particles.

In another embodiment, the fluid sample purification system comprises a distal retainer material that provides both high structural strength and satisfies user requirements such as larger pore size and high flow rate, size, and chemical characteristics.

In another embodiment, the fluid sample purification system comprises a pipette tip that integrates particles and retainer (or filter) while simultaneously minimizing sample dead volume, total internal air volume and DNA adsorption.

In another embodiment, the proximal retainer is a proximal filter.

In another embodiment, the proximal retainer is a pierceable foil seal or cap.

In another embodiment, the undesirable material is preferentially bound to the functionalized particles relative to the molecule of interest.

In another embodiment, the preferential adsorption is on the basis of size, hydrophobicity, charge or affinity.

In another embodiment, the target material can be either a desirable or undesirable material, such as a single-stranded nucleic acid, double-stranded nucleic acid, genomic DNA, plasmid DNA, PCR prime-dimer, nucleotide, ribonucleotide, labeled probe, enzyme, dye, intercalating agents, water, heavy metal, toxin, metabolite, electrolyte, hormone, drugs, antibody or antigen.

In another embodiment, the housing is a pipette tip or column that comprises glass or an inexpensive or commodity plastic. In another embodiment, the inexpensive or commodity plastic is a polyolefin material. In another embodiment, the polyolefin material is polyethylene, polypropylene, polyethylene-terephthalate, polytetrafluoroethylene, or polystyrene.

In another embodiment, the system is disposable.

In another embodiment, the functionalized particles are of sufficiently small size to avoid rapid sedimentation in solution.

In another embodiment, the functionalized particles are of sufficiently large size to enable the use of a high-flow rate filter as the distal retainer and to avoid suspension of the functionalized particles in the air during product manufacturing.

In another embodiment, the distal retainer has sufficiently small pore size to retain the plurality of functionalized particles, while simultaneously sufficiently large to enable a high fluid sample flow rate at a desired applied pressure and avoid damaging the molecule of interest.

In another embodiment, the distal retainer is of sufficient dimensions to provide low tip-to-tip variation in resistance to fluid flow to fluid flow at desired low applied pressures, minimize retained sample volume, and minimize sample dead volume within the pipette tip.

In another embodiment, the distal retainer has sufficiently large pore size that enables low fluid intrusion and extrusion pressures, and sufficiently small pore size to prevent passage of functionalized particles.

In another embodiment, total internal air volume is less than approximately 200 μl.

In another embodiment, total internal air volume is no more than 8-10× the sample volume.

In another embodiment, the system comprises a reaction chamber within the housing and positioned in the space between the distal retainer and the proximal retainer.

Methods for isolating a molecule of interest using fluid sample purification systems are also provided. In some embodiments, the method comprises detecting the isolated molecule of interest.

In one embodiment, the method can comprise:
a. providing a fluid sample to be tested for the presence of the molecule of interest;
b. providing a fluid sample purification system of the invention;
c. drawing the fluid sample into the fluid sample purification system;
d. contacting the plurality of the functionalized particles or adsorption material with the fluid sample; and
e. aspirating and dispensing the fluid sample using an aspirating/dispensing cycle and fluid flow rate that maintains a consistent and consistently high rate of fluid flow, thereby expelling the fluid sample from the fluid sample purification system.

The method can further comprise the step of recovering the molecule of interest from the expelled fluid sample.

In a specific embodiment, a method for isolating a molecule of interest is provided comprising the steps of:
a. providing a fluid sample purification system, wherein the system comprises:
   i. a housing having a distal end with a distal opening adapted for the passage of a fluid and a proximal end with a proximal opening adapted for passage of a fluid;
   ii. a distal retainer inside the housing and above the distal opening;
   iii. proximal retainer, wherein the proximal retainer is located:
inside the housing and between the distal retainer and the proximal opening, or adjacent to, in contact with, covering or sealing the proximal opening; and
   iv. a plurality of functionalized particles, wherein:
the plurality of functionalized particles is inside the housing and confined between the distal retainer and the proximal retainer,
the functionalized particles of the plurality adsorb undesirable material, and
the functionalized particles of the plurality are differentially functionalized particles;
b. drawing a fluid sample into the fluid sample purification system;
c. contacting the plurality of the functionalized particle with the fluid sample; and
d. aspirating and dispensing the fluid sample using an aspirating/dispensing cycle and fluid flow rate that maintains a consistent and consistently high rate of fluid flow;
thereby expelling the fluid sample from the fluid sample purification system.

In one embodiment, a yield of at least 70% of the molecule of interest is obtained.

In another embodiment, a yield of at least 95% of the molecule of interest is obtained.

### 4. BRIEF DESCRIPTION OF THE DRAWINGS

The fluid sample purification system is described herein with reference to the accompanying drawings FIGS. 1-48, in which similar reference characters denote similar elements throughout the several views. It is to be understood that in some instances, various aspects of the invention may be shown exaggerated or enlarged to facilitate an understanding of the invention.

FIG. 1. Table showing the relationship between specific sub-system components and performance benefits for the fluid sample purification system.

FIG. 2. Cross-sectional schematic of a prior art pipette tip.

FIG. 3. Schematic of an embodiment of the fluid sample purification system and method in which DNA is being purified. The left sketch depicts aspiration of target DNA (“blue symbol”), unamplified PCR primers (“black symbols”) and unincorporated nucleotides (“green dots”) in PCR solution into a chamber containing adsorption particles. The expelled solution in the right hand sketch contains target DNA free of impurities (e.g., primers and nucleotides).

FIG. 4. Retention of amplicons (1956 bp), “primer dimers,” (ds 7 bp), single strands (ss 20 bp) and dNTPs from stock solutions from a fluid sample purification using the system of the invention. The order of each set of 4 results bars (% DNA remaining) is B, Blue; R, Red; G, Green; P, Purple. This “BRGP” order is repeated for each entry listed on the X axis.

FIGS. 5a-b. Embodiments of the fluid sample purification system in pipette tip format. (a) Embodiment in which the bead diameter is greater than the distal (nozzle) end of the pipette tip. (b) Embodiment in which the tip has ribs and a plug as anti-clogging features to “engineer” or direct the flow of the fluid sample in the tip housing.

FIGS. 6a-b. An embodiment of the fluid sample purification system in “pipette tip format,” i.e., that comprises a housing that is a pipette tip.

FIG. 7a. An embodiment of the fluid sample purification system that comprises a housing that is a pipette tip and a pierceable seal or cap. This particular embodiment has pre-formed perforations in the seal.

FIG. 7b. An embodiment of the fluid sample purification system that comprises a housing that is a pipette tip and a removable cap.

FIG. 8. PCR purification conducted on single solutions to compare the performance of a prior art (“Competitor Q,” “Competitor I”) purification product with the performance of embodiments of the fluid sample purification system of the invention in pipette tip (“Diffinity tip”) and column (“Diffinity tube”) formats.

FIG. 9. PCR stock solution was prepared from purified PCR amplicons and impurities (462 ng/μl dNTP, 2.4 ng/μl primer). This stock solution was then subjected to PCR purification with either a prior art product (Q, I, and E) or an embodiment of the fluid sample purification system (functional tip or spin column).

FIG. 10. Sanger sequencing is a quantitative functional test of purified PCR solutions. Unpurified simulated PCR solution was purified with several competitors' products (Q, I, and E) as well as with the Diffinity tip and spin column according to manufacturer's protocols. Left: Two quantitative measures of sequence quality, the continuous read length (CRL) and quality value (QV20) are plotted above with each column representing the average of two identical, independent sequence reactions. Longer, higher quality read lengths have higher values while unpurified sample acts as a negative control and has lower values. Right: Sequence chromatograms from the middle region of the available sequence visually show an improvement in peak quality between unpurified and most purified samples as exhibited with an improved signal-to-noise ratio. These results are representative of those observed in more than 5 similar experiments.

FIG. 11. Comparison of sequence after cleanup with a control system and an embodiment of the fluid sample purification system. Independently run beta tests were conducted at 5 sites to compare an embodiment of the fluid sample purification system in pipette tip format (“Diffinity functional PCR purification tip”) to methods currently used by the respective testing labs. PCR samples were divided to be used with either control (80.92+/−6.59%) or Diffinity (80.99+/−6.51%) PCR Purification. After sequencing, the continuous read length (CRL) was normalized as a percentage of the expected read length and averaged for each group. Shown is the average of 30 trials with standard error bars.

FIGS. 12a-c. Typical sample aspirate and dispense pressures within a commercially available Thermo Fisher Scientific Aspire™ pipette tip (“Prior Art Tip”) as it aspirates and dispenses water.

FIGS. 13a-c. Typical sample aspirate and dispense pressures within a pipette tip of the fluid sample purification system as it aspirates and dispenses water.

FIG. 14. Embodiment of the fluid sample purification system in which the housing is one or more fluid ducts (e.g., columns or capillary tubes; Capillary #1, Capillary #1+M (integer), Capillary #N (integer), etc.). Section A-A shows a cross-section of multiple nested fluid ducts. Capillary radius r indicated at distal end at lower right.

FIG. 15a. Schematic of non-circular support used in one embodiment of the fluid sample purification system. In this particular example, the housing is a pipette tip and the width of the support (show as “Minimum Support Width”) is wider than the diameter of the pipette orifice.

FIG. 15b. Schematic of an embodiment of the fluid sample purification system wherein a spherical particle is retained by a non-circular (elliptically shaped) pipette nozzle. In this particular embodiment, the minimum diameter of the particle is greater than the maximum length of the minor axis of the elliptically shaped nozzle.

FIG. 16. Calculations for predicted particle settling times using methods described by Bird, Stewart and Lightfoot (1960, Transport Phenomena, John Wiley & Sons, ISBN 047107392X).

FIGS. 17a-d. Fluid aspirate and dispense cycle times from pipette tips that were loaded with either 2.5 or 10.0 milligrams of unfunctionalized particles with a hydrophobic surface. Fluid was then aspirated and dispensed from the tips.

FIGS. 18a-d. Experiments showing the results of particles forced against the surface of a distal retainer (filter) during the sample dispense cycle in an embodiment of the fluid sample purification system in pipette tip format. Particles sized at less than 53 μm, 53-74 μm, 74-100 μm and 105-149 μm create maximum dispense pressures of 3.4 KPa, 3.1 KPa, 3.0 KPa and 2.5 KPa, respectively.

FIG. 19. Schematic of an embodiment of the fluid sample purification system in pipette tip format comprising distal and proximal retainers for retaining the particles, beads or other adsorption materials. In the schematic, a proximal retainer and a distal retainer are shown. (a) is an enlarged view of the pipette tip showing the distal retainer.

FIG. 20a. Schematic of an embodiment of the fluid sample purification system in pipette tip format comprising distal and proximal retainers for retaining the particles, beads or other adsorption materials. In the schematic, a proximal porous matrix retainer and a distal porous matrix retainer are shown. At left is an enlarged view of the pipette tip showing the distal retainer.

FIG. 20b. Schematic of an embodiment of the fluid sample purification system in pipette tip format showing the use of a retention element (rib) integral to the housing for retaining the distal retainer.

FIG. 20c. Schematic of an embodiment of the fluid sample purification system in pipette tip format showing the use of a retention element (flap) integral to the housing for retaining the distal retainer.

FIG. 20d. Schematic of an embodiment of the fluid sample purification system in pipette tip format showing the relationship of particle void volume to pipette tip free volume, wherein free volume≧particle void volume.

FIG. 21. An embodiment of the fluid sample purification system that comprises a retention element (rib) integral to the housing for retaining the internal filter(s).

FIG. 22. Another embodiment of the fluid sample purification system that comprises a retention element (rib) for the distal retainer (filter).

FIG. 23. An embodiment of the fluid sample purification system in pipette tip format. The retainer(s) can be held in place within the housing using internal raised elements, such as barbs, which utilize mechanical interference to secure the retainer.

FIG. 24. An embodiment of the fluid sample purification system in which a filter material (e.g., ceramic) is used that provides the structural strength required to reliably maintain pore size. The figure illustrates the high forces placed upon filters and hence, the need for high filter structural strength.

FIG. 25. An embodiment of the fluid sample purification system in pipette tip format. Pipette tip ‘A’ includes insert ‘B’ of thickness ‘t’ positioned within pipette tip ‘A’ by insert positioning tabs ‘C’ creating fluid flow conduits ‘D’ of width ‘E’. Cross-section A-A through pipette tip ‘A’ illustrates fluid flow through the axially-running fluid flow conduits ‘D’ formed by the space between pipette tip ‘A’ and insert ‘B’. Conduit width ‘E’ is precisely determined by the recess in insert ‘B’, such as for example, by the injection mold that precisely forms this feature of the part during the pipette tip injection molding process. The width ‘E’ of flow conduits ‘D’ are chosen to minimize fluid flow resistance, while in one application preventing larger objects (e.g. particles) from passing into or out of pipette tip ‘A’.

FIG. 26. An embodiment of the fluid sample purification system in pipette tip format. Pipette tip ‘A’ includes insert ‘B’ of thickness ‘t’ positioned within the pipette tip by insert positioning tabs ‘C’ creating fluid flow conduits ‘D’ of width ‘E’. Unlike the embodiment of the fluid sample purification system shown in FIG. 25, the flow conduits are created in the inner wall of pipette tip ‘A’. Cross-section A-A through pipette tip ‘A’ illustrates fluid flow through the axially-running fluid flow conduits ‘D’ formed into the wall of pipette tip ‘A’. Conduit width ‘E’ is precisely determined by the “core” of the pipette tip injection molds that precisely forms the internal surfaces of the pipette tip ‘A’ during the pipette tip injection molding process. As before, the width ‘E’ of flow conduits ‘D’ are chosen to minimize fluid flow resistance, while in one application preventing larger objects (e.g. particles) from passing into or out of pipette tip ‘A’.

FIG. 27. Absence of DNA damage due to small filter pore size. DNA treated with an embodiment of the fluid sample purification system in pipette tip format (“Diffinity RapidTip” embodiment) shows no evidence of shear damage after either 15 or 30 aspiration-dispense cycles.

FIGS. 28a-f. Schematics of embodiments of the fluid sample purification system in pipette tip format comprising particles or beads in various formats. (a) Bead macro-package. (b) Bead micro-package. (c) Singe (large) bead volume. (d) Multiple (small) bead volume. (e) Multiple (small) bead volumes with multiple adsorption agents, one agent per bead volume. (f) Multiple (small) bead volumes with multiple adsorption agents on each bead volume.

FIG. 29. Embodiment of the fluid sample purification system in pipette tip format in which an adsorption agent is applied to or coated on the inner surface of the housing (i.e., the pipette tip).

FIG. 30. Embodiment of the fluid sample purification system in pipette tip format in which an adsorption agent is incorporated into a compressed thin film or into filaments and inserted inside the housing.

FIG. 31. Embodiment of the fluid sample purification system in pipette tip format in which one or more features are added to enhance the internal wetted surface area of the housing (pipette tip). The adsorption material or agents are attached to multiple features within the pipette tip that serve to increase wettable surface area, e.g., multiple, internal, axial running ribs, such as those shown in this figure. Section A-A is a cross-sectional view of the features, which in this embodiment are surface area enhancing ribs.

FIG. 32 An embodiment of the fluid sample purification system that accommodates filters with larger cross-sectional area and hence has lower resistance to fluid flow, while simultaneously minimizing sample dead volume. The pipette tip has a small bore at the pipette nozzle and rapidly increasing the internal diameter of the pipette tip to accommodate the larger filter.

FIG. 33. Embodiment of the fluid sample purification system comprising a vessel and adsorption materials or agents in the sample solution. According to this embodiment the adsorption material or agents are placed upon beads or particles that are either added to a vessel containing the sample to be purified, such as a test tube or PCR reaction vessel, or contained in the vessel prior to adding the sample fluid, whereby they are allowed to attach to materials to be removed from the solution.

FIG. 34. Embodiment of the fluid sample purification system in which the adsorption material or agents are attached to the surface of a film which is placed inside the housing (e.g., pipette tip). Preferably, the film is rolled up into a column (e.g., a spiral) so as to provide a large film surface area. The diameter of the coil is preferably larger than the diameter of the pipette nozzle so as to contain the film within the pipette tip. Section A-A shows a cross-sectional view of the coil.

FIG. 35. Embodiment of a “porous media retainer,” i.e., a fluid sample purification system in tube (here a capillary tube) or column format. Adsorption material or agents can be attached to a high surface area material, such as the internal and/or external surfaces of a volume or volumes of a porous media. The sample fluid to be purified is then brought into contact with the distal end of the porous media retainer.

FIG. 36. Embodiment of the fluid sample purification system comprising a self-contained purification package. According to this embodiment, adsorption material (e.g., particle)-containing package is inserted in the sample fluid and agitated, such as by the use of stirring or repeated insertion and removal, to enhance the movement of fluid through the sample vessel, thereby enhancing the likelihood of encountering materials to be adsorbed, thereby increasing its adsorption efficiency and reducing the time required to do so.

FIG. 37. DNA retention (adsorption) by various types of filter material.

FIG. 38. DNA retention by various filter pore size.

FIG. 39. Interaction of various commercially available plastic pipette tips with DNA. Pipette tips were obtained from several suppliers and used to repetitively aspirate and dispense DNA solution for 90 seconds. DNA concentration was measured using the absorbance at 260 nm before and after treatment and is reported as the % initial ([ ]after treatment/[ ] before treatment*100). Data is shown as the,average of three replicates with error bars showing standard deviation.

FIGS. 40a-b. (a) A conventional pipette tip design with typical pipette tip dimensions. (b) An embodiment of the fluid sample purification system comprising a pipette tip with a reaction (or mixing) chamber, and showing that the dimensions of this pipette tip design create an internal slenderness ratio (i.e., the ratio of height to diameter) that is preferably approximately 1.0.

FIG. 41. Embodiment of the fluid sample purification system in pipette tip format that minimizes pipette tip residual volume. The distal retainer (or filter) minimizes, in the wetted housing (pipette tip), the volume of fluid that is retained between the distal end of the distal retainer and the distal end of the pipette tip, as shown by dimension “X.” Preferably, the particle retainer is positioned axially such that this “dead volume” is no larger than 10% of the total volume of sample fluid aspirated into the pipette tip.

FIG. 42a. Representative mixing protocols used to achieve successful purification. All experimental conditions were identical except the mixing protocol. Protocol C has faster, more vigorous mixing which results in the fastest purification time.

FIG. 42b. Protocol changes affect purification time. Trial A, B, and C used identical DNA solutions and embodiments of the fluid sample purification system in pipette tip format with the only difference being the mixing protocol. DNA concentration was measured during treatment by the absorbance at 260 nm and normalized as a percentage of the pre-treatment concentration. For each trial, a dotted vertical line marks the time required to achieve adequate purification (<10% impurities remaining). As protocol improvements were made, the purification time drops from 2 minutes in trial A to 75 seconds in trial B and finally to 45 seconds in trial C.

FIG. 43. Binding kinetics for functionalized particles confined in the “large bore” Pipette tip shown in FIG. 20b. 2.5 mg of functionalized particles was loosely inserted into a pipette tip and 25 μl of DNA solution was treated using a pipettor volume of 50 μl.

FIG. 44. An embodiment of the fluid sample purification system comprising a low internal volume functional pipette tip.

FIG. 45. An embodiment of the fluid sample purification system comprising a filtration column and a cap.

FIG. 46. Another embodiment of the fluid sample purification system comprising a filtration column, integrated reagents and a cap.

FIGS. 47a-b. Performance data from two tests of the embodiment of the fluid sample purification system discussed in Section 6.1 (Example 1). The system shows very consistent fluid aspirate and dispense pressures over multiple aspiration and dispense cycles.

FIG. 48. A summary table of fluid shear rates for a wide range of pipette tip nozzle or particle retainer perforation diameters (columns) and fluid flow rates (rows). See Section 5.7 (pipette nozzle and particle retainer orifice sizes and fluid dispense velocities) for details.

### 5. DETAILED DESCRIPTION OF THE INVENTION

Systems, devices and methods for purifying fluids are provided that use adsorption materials. Systems, devices and methods are also provided for containing and manipulating fluids to be purified.

In one embodiment, a fluid sample purification system for isolating a molecule of interest in a fluid sample is provided. The fluid sample purification system comprises a housing having a distal end with a distal opening adapted for the passage of a fluid and a proximal end with a proximal opening adapted for passage of a fluid; a distal retainer inside the housing and above the distal opening; proximal retainer, wherein the proximal retainer is located inside the housing and between the distal retainer and the proximal opening, or adjacent to, in contact with, covering or sealing the proximal opening; and a plurality of functionalized particles.

In one embodiment, the housing is pipette tip.

In another embodiment, the housing is a column or tube (e.g., capillary tube).

In another embodiment, the fluid sample purification system comprises a housing (e.g., a vessel or tube) but has neither a distal or proximal retainer. One or more openings in the housing can be adapted for the passage of fluid.

Fluids can be in a liquid or a gaseous form. According to the invention, any fluid can be purified, whereby components in the fluid can be distinguished by size, charge, hydrophobicity or hydrophilicity, or affinity for other molecules.

Also provided arc methods for collectively optimizing and integrating adsorption-system components to provide a fluid sample purification system that achieves a level of system performance that would not otherwise be attainable without such component-level and system-level optimization and integration. According to the invention, a greater level of system-level performance is achieved through optimization and integration of multiple, inter-dependent, sub-system components to meet often conflicting design constraints.

In one embodiment, the fluid sample to be purified by the fluid sample purification system and methods of the invention is a biological fluid. A biological fluid is a fluid located within, or produced by, an organism or a component of an organism. Major components of organisms can include, but are not limited cells and macromolecules. Cells can include but are not limited to eubacteria, archaea and eukaryotes. Sub-cellular components of an organism can include, but are not limited to nuclei, mitochondria and chloroplasts, endoplasmic reticulum, and microbodies. Components that are macromolecules can include proteins and nucleic acids, polysaccharides, lipids and complex assemblies of macromolecules, such as nucleoproteins (i.e., associations of nucleic acids and proteins) and glycoproteins (i.e., associations of protein and carbohydrates). Nucleic acids can be any known in the art, for example, deoxyribonucleic acid (DNA) including plasmids; and ribonucleic acid (RNA) including mRNA, tRNA, rRNA, snRNA, other non-coding RNA, PCR primers, nucleic acid probes, etc. Proteins can any known in the art, for example, polypeptides, peptides, amino acids, and antibodies.

The invention also provides systems and methods for purifying other types of fluid samples.

The fluid sample purification methods of the invention can be distinguished from currently available particle-filtration based purification processes used, for example for purifying nucleic acids in that these other purification processes adsorb both the target nucleic acid and impurities and take advantage of the differential solubility of the desired target and impurities by utilizing a series of chemical-mechanical processes to selectively and sequentially elute impurities and desired target before passing either impurities and/or desired target through a filtration membrane. By comparison, the fluid sample purification system works by adsorbing only the impurities and allowing the desired target to pass through the fluid sample purification system in a single step.

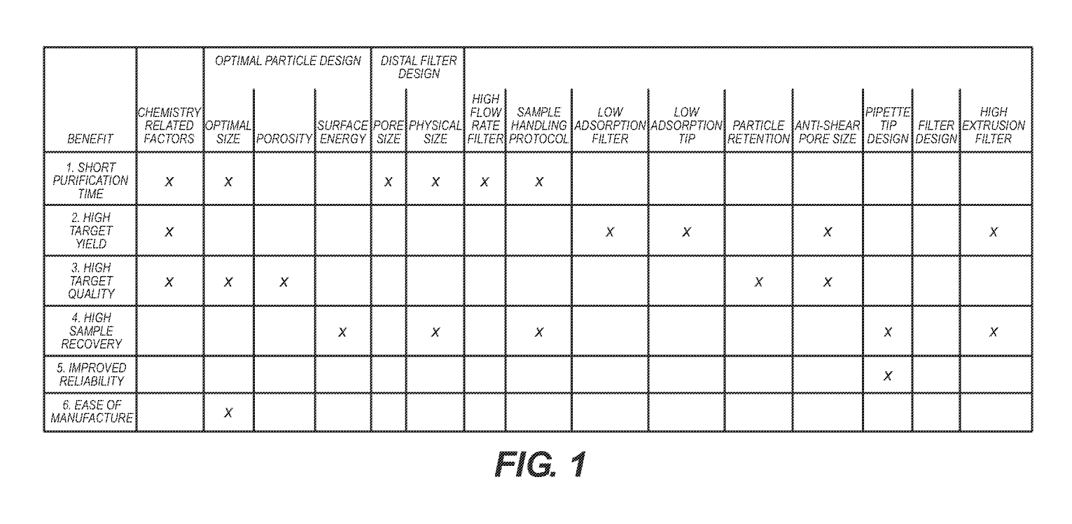

The benefits achieved by this component-level and system-level optimization include very short sample processing time, high target yield, high target quality, increased adsorption capacity, high sample volume recovery, improved reliability of operation and ease and cost of product manufacturing. The relationship between specific sub-system components and system performance benefits is shown in FIG. 1.

In contrast to the existing technologies and products described above and known in the art, the present fluid sample purification system, in one embodiment, uses only a single disposable and single processing step and no addition or subsequent removal of reagents. This single-step method reduces the potential for operator error and results in a very rapid, easy to use and low cost process. When applied to the purification of DNA from PCR solutions, for example, the process takes approximately 30-60 seconds to complete (versus 15-30 minutes for existing technologies and products) and removes over 90% of the impurities (i.e., unincorporated primers and nucleotides) while leaving ample target DNA for subsequent analysis. Additional system performance benefits are described later in this document.

Preferably, the adsorption material selectively adsorbs undesirable materials while simultaneously rejecting desirable materials during a single exposure to the sample solution (a single “exposure” can include multiple fluid aspiration and dispense cycles).

In one embodiment, the undesirable material is preferentially bound to, attracted to or adsorbed by the functionalized particles relative to the molecule of interest, owing to differences in, e.g., size, hydrophobicity, charge or affinity.

The differentially adsorbed (or rejected) material, depending upon the application, can be, for example, single-stranded nucleic acids such as RNA or PCR primers, double-stranded nucleic acids, such as DNA (e.g., genomic DNA, plasmid DNA) or PCR primer-dimers, nucleotides such as ribonucleotides, enzymes such as DNA polymerases, RNA polymerases, labeled probes, restriction enzymes or reverse transcriptases, dyes such as intercalating agents, water, heavy metals, toxins such as blood-borne toxins (e.g., urea or creatinine), blood components including metabolites, electrolytes, hormones or drugs, and antibodies or antigens.

Table 1 describes various exemplary applications in which the systems and methods for fluid sample purification can be employed.

TABLE 1

| Life Science Application | Adsorbed materials | Rejected materials |
|---|---|---|
| PCR Cleanup | Nucleotides | ds-DNA (PCR |
| | Primers | amplicons) |
| | Primer-dimers | |
| | Polymerase | |
| Genomic DNA | RNA | Genomic DNA |
| Extraction | Proteins | |
| | Cellular debris | |
| Plasmid Prep | Genomic DNA | Plasmid DNA |
| | RNA | |
| | Protein | |
| | Cellular debris | |
| RNA Purification | DNA | Target RNA |
| | Protein | |
| | Cellular debris | |
| Gel Extraction | Agarose | ds-DNA (PCR |
| | Ethidium bromide | amplicons) |
| | Intercalating dye | |
| Next-Gen Sequencing | Primers/Primer-dimers | ds-DNA (PCR |
| Barcoded Samples | Nucleotides | amplicons) |
| | Enzymes | |
| Next-Gen Sequencing | Primers/Primer-dimers | ds-DNA (PCR |
| Library prep | Nucleotides | amplicons) |
| End repair | Enzymes | |
| 3′A addition | Water (sample | |
| Adaptor ligation | concentration) | |
| PCR/Size selection | | |
| Restriction Digest | Short ds-DNA fragments | ds-DNA (Plasmid) |
| Purification | Restriction enzymes | |
| In vitro Transcription | Ribonucleotides | RNA |
| | RNA polymerase | |
| Reverse Transcription | Reverse transcriptase | ds-DNA |
| PCR | Primers/Primer-dimers | |
| | Nucleotides | |
| | Polymerase | |
| Molecular diagnostics | Unincorporated labeled | Target containing |
| | nucleic acid probe | incorporated nucleic |
| | | acid probe |
| Dialysis | Urea | All other blood- |
| | Creatine | borne components |

Table 1 describes various exemplary applications in which the systems and methods for fluid sample purification can be employed.

Many amplification, purification, diagnostic or assay methods for biomolecules can employ the fluid sample purification system and methods of the invention, including, but not limited to PCR cleanup, genomic DNA extraction, plasmid preparation, RNA purification, gel extraction, next-gen sequencing (e.g., barcoded samples, library preparation, end repair 3′ addition, adaptor ligation, PCR/size selection), restriction digest purification, in vitro transcription, reverse transcription, PCR, molecular diagnostics, dialysis (see Table 1). To achieve selective removal of contaminants from reactions in such methods or assays, the pores of the nanoporous materials can be coated so as to strongly adsorb a material while the exterior of the nanoporous materials (the non-pore surface) is made resistant to adsorption of the material. This results in spatially inhomogeneous functionalization of the materials, which is generally intended to mean that the nanoporous material comprises surfaces that bear at least two distinct functional groups.

In one embodiment, nanopores are functionalized with different materials providing some functionalized nanopore surface which is effective at removing unincorporated nucleotides (dNTP) and primers. An example of such a material is positively charged or hydrophobic compounds. The positively charges or hydrophobic compounds adsorb contaminates, such as dNTPs, primers, ssDNA, and the like. To impart a positive charge to the surface of the pores, amine or metal ion containing compounds can be used. An example of an amine containing compound is aminopropyltrimethoxysilane (APTMS). The positive charge can be introduced using silanization chemistry (e.g., reaction with amino-silanes such as APTMS) or self-assembly of positively charged polyelectrolytes with low molecular weight (e.g., poly allylamine, polyethyleneimine or polylysine).

According to this embodiment, the external (non-nanopore) surface of the material can be functionalized with compounds that resist adsorption of ds nucleic acids, e.g., DNA PCR amplicons, which diffuse poorly into the nanopores. Suitable adsorption resistant materials can include negatively charged or hydrophilic compounds, or any compound known to be resistant to adsorption of biomolecules (e.g., polyethylene glycol). Suitable negatively charged compounds include but are not limited to anions, polyanions, polyelectrolytes, carboxylate, oligoethylene glycol, hydroxide compounds, and combinations thereof.

One useful aspect of the fluid sample purification system is the ability of the adsorption material to simultaneously serve as an exclusion material, i.e., undesirable materials in a solution are adsorbed while desirable materials in the same solution are simultaneously repelled (rejected) in a single step.

In various embodiments, a fluid sample purification system is provided that uses system and sub-system component designs and materials for preferentially adsorbing various types of NA, such as for example, the removal of impurities such as oligonucleotides and single-stranded primers from a PCR solution, while simultaneously rejecting the desired PCR product (i.e., double-stranded DNA amplicons). This enables purification of samples to take place in a single step. This is unlike existing products and technologies, in which impurities, as well as target materials (e.g., target DNA), are adsorbed onto adsorbing surfaces, and that require multiple, complex, and time-consuming steps to remove impurities and release the desired target DNA.

In a preferred embodiment, a fluid sample purification system is provided that comprises a housing that is a functional, disposable pipette tip that fits on any single or multiple, manual or automated, laboratory pipettor. For many NA purification applications, this general design embodiment achieves in a single pipette tip all of the purification capability that existing purification products achieve through the use of multiple disposables, multiple reagents, and complex mechanical-chemical processes.

The fluid sample purification system can be packaged as a kit. Such a kit can comprise, for example, a housing (e.g., a pipette tip or capillary tube), adsorption material (contained within the housing or in a separate container), and a user protocol. The kit can also comprise one or more (integrated) retainers or filters or suitable reagents for the desired fluid sample purification. In another other embodiment, the kit can comprise adsorption material, retainers and a user protocol, with the end-user typically providing fluid handling consumables (e.g., housings such as pipette tips) and commonly used reagents.

The manner in which these components are optimized and integrated together creates a superior-performing fluid sample purification system that satisfies many competing sub-system performance requirements. A series of system and sub-system component design concepts, optimal system and sub-system component design parameters, and means of integrating the sub-system components described hereinbelow together to achieve superior-performing fluid sample purification systems are described.

For clarity of disclosure, and not by way of limitation, the detailed description of the invention is divided into the subsections set forth below.

### 5.1 Pipette Tips and Fluid Sample Purification Systems Comprising Pipette Tips

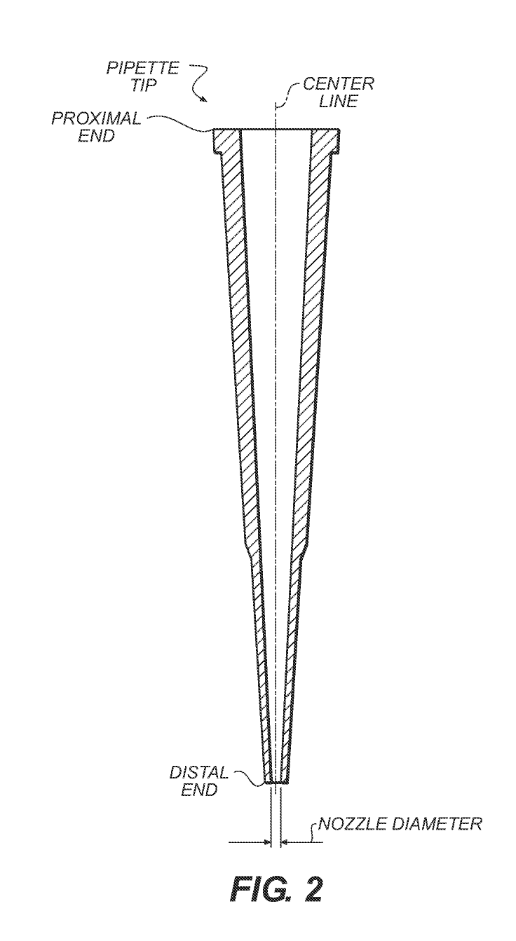

A fluid sample purification system is provided. In one embodiment, the fluid sample purification system can comprise a pipette tip that fits on any conventional single or multiple, manual or automated, laboratory pipettor. A typical prior art pipette tip design is shown in FIG. 2. In other embodiments, the fluid sample purification system can comprise a component in column format rather than in pipette tip format.

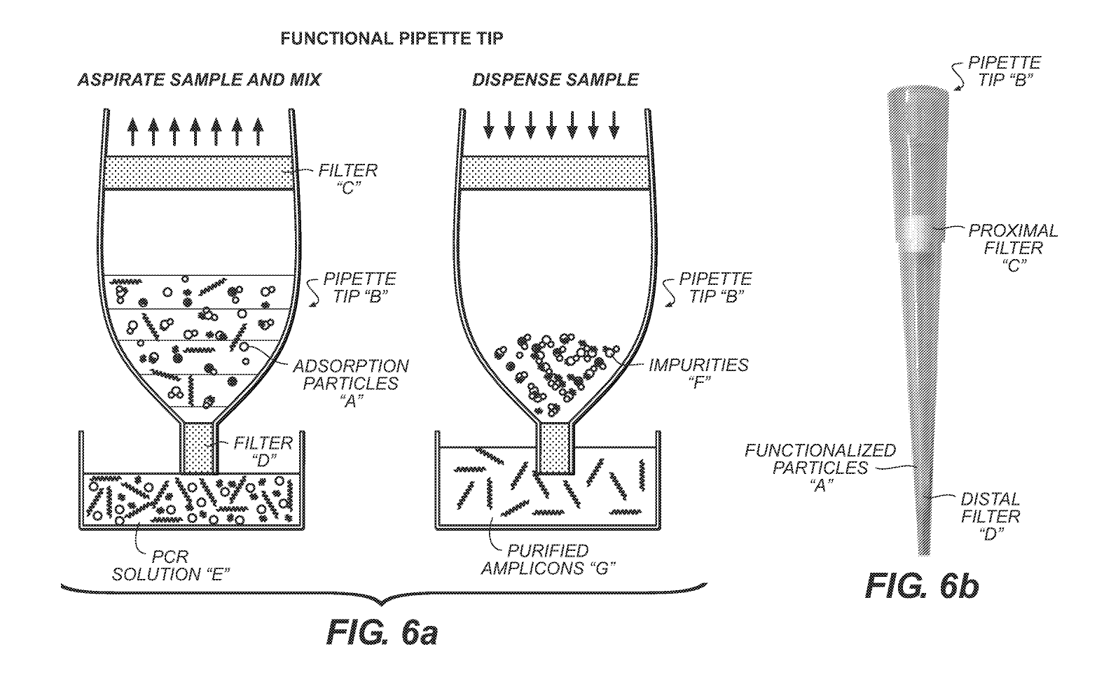

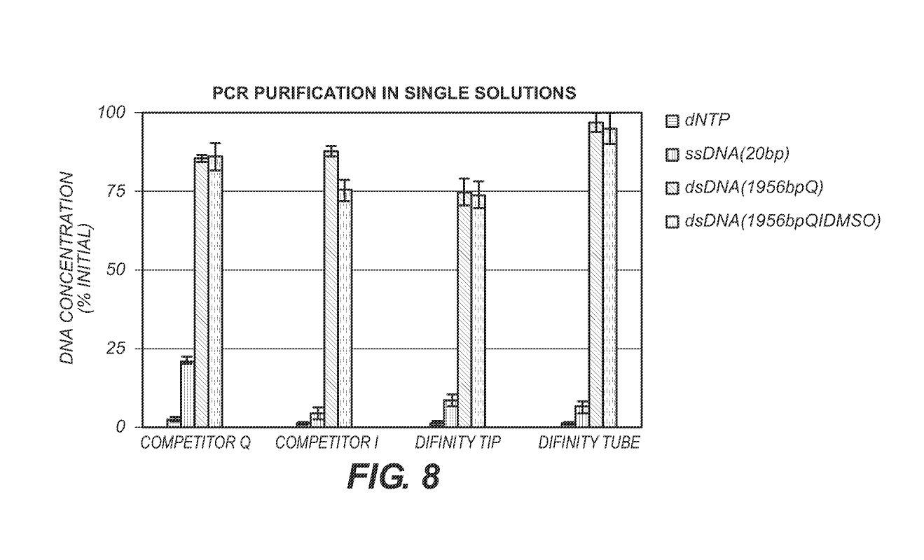

In one embodiment in which the fluid sample purification system comprises a pipette tip, the tip can be packed with particles containing adsorption materials (FIG. 6). To use this embodiment, a user aspirates into the pipette tip the solution to be processed, such as a PCR solution containing amplified double-stranded DNA and impurities. The impurities, such as unincorporated single-stranded primers and oligonucleotides, are adsorbed by the particles within the pipette tip. Dispensing the solution from the pipette tip yields solution containing purified double-stranded DNA, the desired PCR amplified product. Solution purification can be accomplished using handheld pipettors that are standard equipment in most labs or with standard robotic liquid handlers used in high throughput applications. Results demonstrating efficacy of the process are presented in FIGS. 8, 9, 10 and 11.

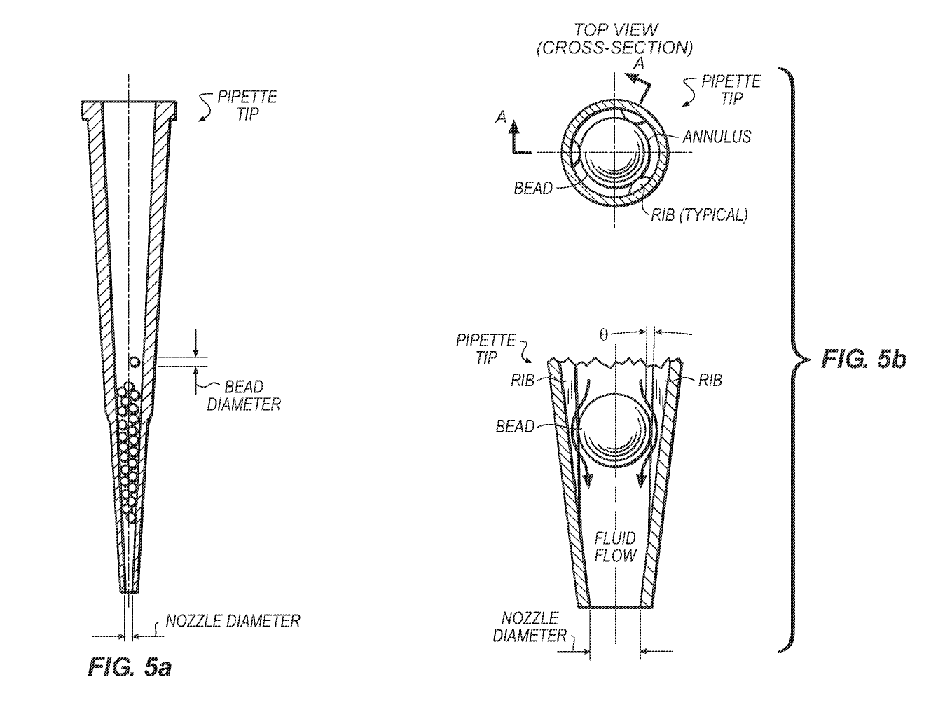

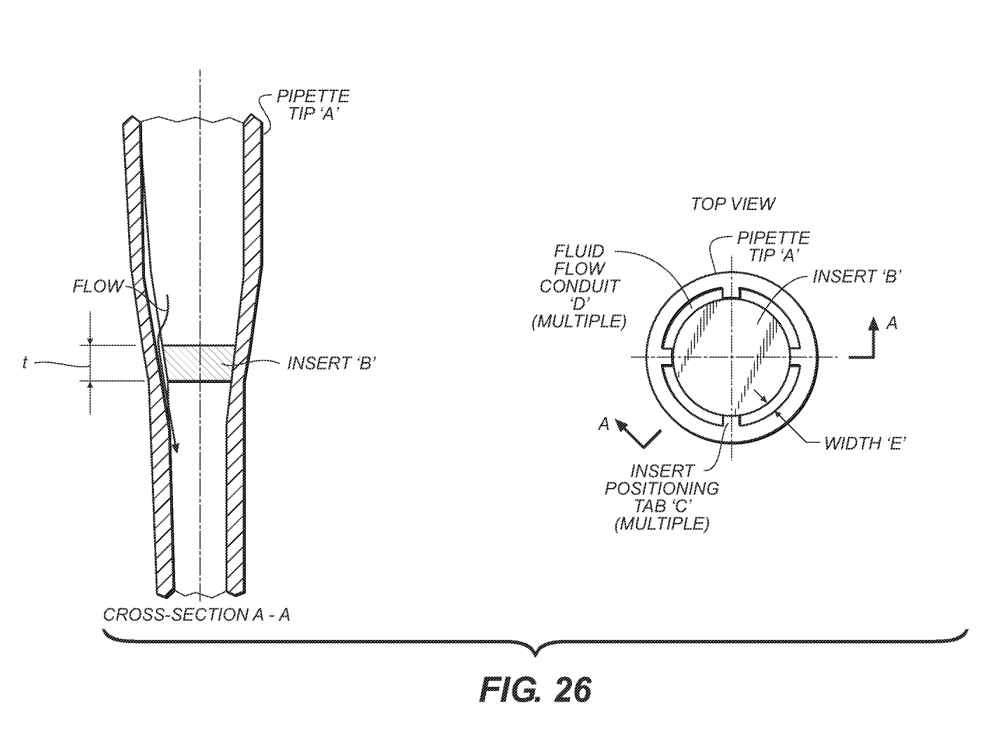

FIGS. 5a-b and FIG. 26 show embodiments of the fluid sample purification system in pipette tip format. In FIG. 5a, the bead diameter is greater than the distal (nozzle) end of the pipette tip. In FIGS. 5b and 26, the tip has ribs and a plug as anti-clogging features to “engineer” or direct the flow of the fluid sample in the tip housing.

Internal, integral pipette features, such as the axially running ribs shown in FIG. 5b can be used to contain particles within the housing while simultaneously allowing fluid to pass around the exterior of the particle, into and out of the housing. The ribs may be, for example, a feature molded into the housing or a separate part that is inserted into the housing to provide the same particle retention function. This latter feature can provide the additional advantage of preventing spherically or near-spherically shaped particles from plugging the nozzle of the pipette tip or other housing. These same axially running ribs can also be used to establish the fluid flow duct shown in FIG. 26.

In a specific embodiment, the adsorption materials are immobilized on particles contained within (or packed into) the pipette tip. The particles can be free to move about within a pipette tip or they can be confined or constrained to one or more regions within the pipette tip.

In another embodiment, the interior surface of the housing fluid sample purification system comprises a differentially functionalized surface (e.g., a coated capillary tube for processing extremely small sample volumes).

In another embodiment, the adsorption materials preferentially adsorbs single- or double-stranded DNA, primers, oligonucleotides, other biological materials or other reagents involved in the purification of nucleic acids, proteins or other biomolecules.

In one preferred embodiment, a fluid sample purification system is provided that comprises a pipette tip packed with “functionalized” particles comprising one or more adsorption materials (FIG. 6a). Adsorption materials A, in this case particles, are packed into the distal end of pipette tip B and are retained within the pipette tip by filters C and D. Filter C is positioned near the proximal end of the pipette tip where it serves to retain the particles in the pipette tip. Filter D is positioned near the distal end of the pipette tip to retain the particles and also to minimize the volume of fluid contained between the pipette tip nozzle and (distal) end of filter D. A photograph of a representative product is shown in FIG. 6b.

FIG. 6a also shows an embodiment of the fluid sample purification system. A user aspirates into the pipette tip the solution to be purified, such as a PCR solution E containing amplified double-stranded DNA and impurities. The impurities, such as single-stranded primers, primer-dimers and oligonucleotides, are adsorbed onto the particles within the pipette tip. Dispensing the solution from the pipette tip yields a solution containing purified double-stranded DNA G, the desired PCR amplified product, in a single step. Impurities F are adsorbed onto adsorption materials A which are retained within the pipette tip B by distal filter (retainer) D whose pore size is chosen to freely pass the sample solution but not the adsorption particles containing the impurities. The solution may be aspirated and dispensed from the pipette tip several times to help ensure adequate interaction between the solution and adsorption particles. Solution purification can be accomplished using handheld pipettors that are standard equipment in most labs or with standard robotic liquid handlers used in high throughput applications.

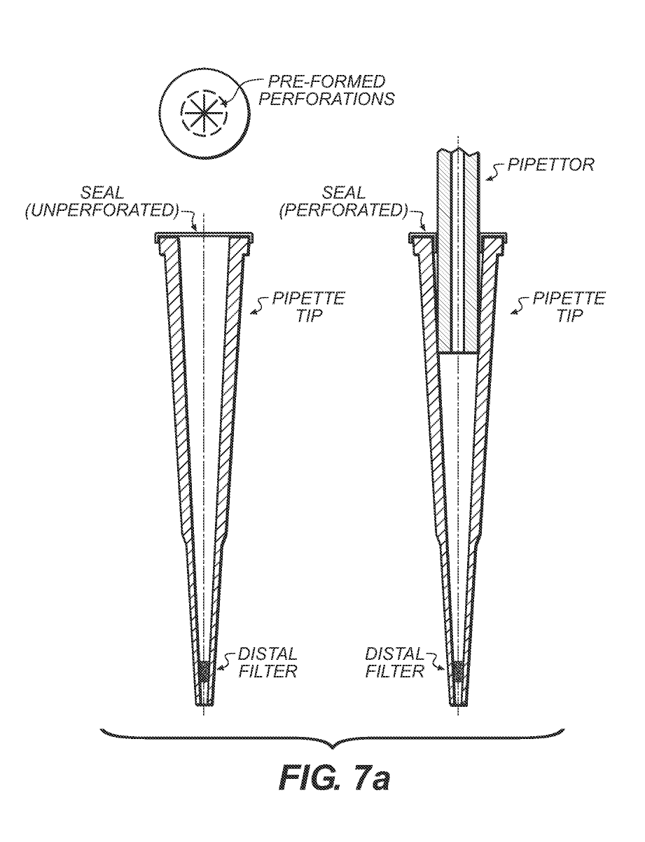

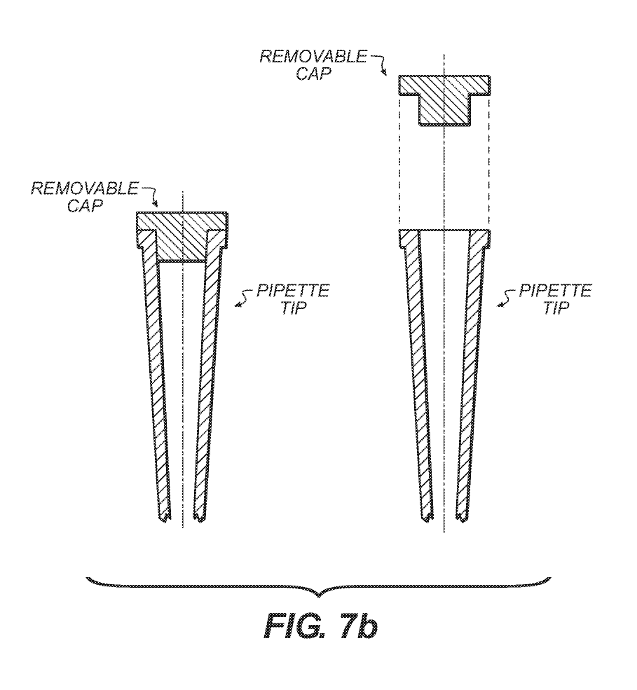

FIG. 7b shows another embodiment in which proximal filter C is replaced by a removable seal at the proximal end of the pipette tip.

FIG. 7a shows another embodiment in which the fluid sample purification system comprises a seal at the proximal end of the pipette tip. The seal preferably comprises a thinner, non-shedding material that can be easily pierced (e.g., by the pipettor). Such materials for seals are well known in the art and can be, for example, a metal foil, a polymer membrane or a tightly woven fabric or paper material. The seal may or may not be pre-scored so as to help ensure proper perforation and/or piercing.

To achieving a consistently high level of product performance, optimization and integration of system and sub-system components can be carried out as described hereinbelow. Although the optimization and integration of the sub-system components is discussed in a serial fashion, it will he understood by the skilled practitioner that these activities can occur in parallel, as is typically the case with system design optimization. The choice of component design concept and optimal design parameters values frequently involve making tradeoffs between competing product performance requirements.

The characteristics of pipette tips of the fluid sample purification system include the number and type of filters used, reaction chamber design, adsorption material design (e.g., type, size and volume of adsorption particles), sample dead volume, total internal air volume, target adsorption characteristics, pipettor compatibility, and other design features disclosed herein.

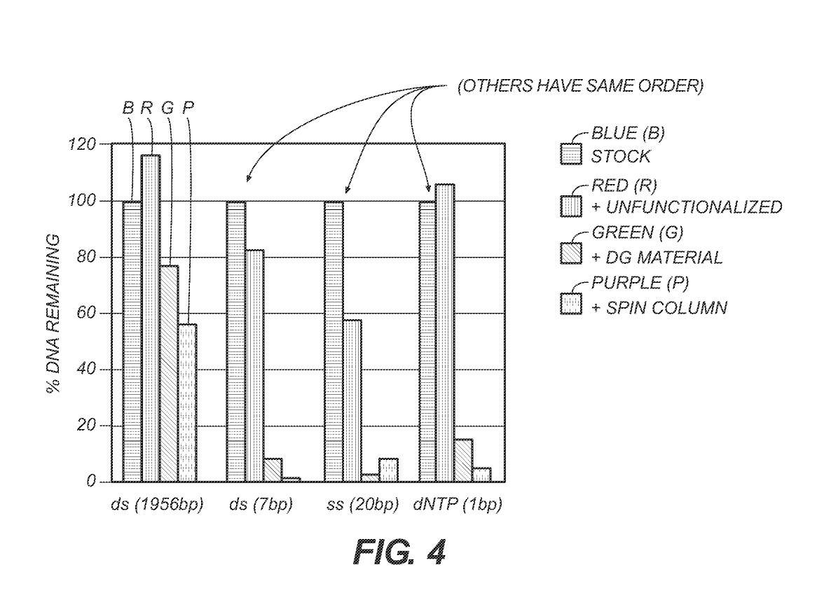

FIG. 8 demonstrates experimental performance of embodiments of the fluid sample purification system. PCR purification was conducted on single solutions to compare the performance of a prior art (“competitor”) purification product with the performance of embodiments of the fluid sample purification system of the invention (“Diffinity”) (FIG. 4). DNA absorbance (at 260 nm) was measured using the Nanodrop 1000 before and after PCR purification. PCR purification was performed according to the manufacturer's protocol. DNA solutions were treated in the Diffinity tip (i.e., pipette tip) for 75 seconds and in the Diffinity tube (i.e., column) for 30 seconds. DNA concentration is shown as % initial concentration where a theoretically perfect purification product that removed all impurities and retained all target amplicon would have 0% dNTP, 0% single stranded DNA (ssDNA), and 100% double stranded DNA (dsDNA). Each bar represents the average of 3 separate experiments+/−standard error of the mean. Enzymatic purification methods are not shown due to their incompatibility with absorbance measurements of concentration. In additional experiments, target yields up to 95±5% of initial DNA concentration have been obtained in the fluid sample purification system in pipette tip format (“Diffinity tip”) (data not shown).

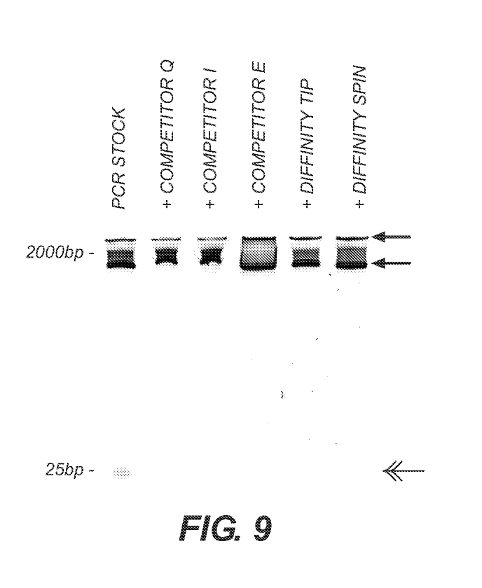

In FIG. 9, a PCR stock solution was prepared from purified PCR amplicons and impurities (462 ng/μl dNTP, 2.4 ng/μl primer). This stock solution was then subjected to PCR purification with either a prior art product (Q, I, and E) or an embodiment of the fluid sample purification system (functional tip or spin column). Manufacturer's protocols were followed and equivalent amounts of purified and stock PCR were run on adjacent lanes of a 5% non-denaturing polyacrylamide gel. The black arrow points to PCR template (lambda genomic DNA), the grey arrow points to the PCR amplicon (1956 bp band) and the double arrow points to the primer (24 bp). Distorted amplicon bands are visible in lanes purified with either competitor Q or I; the distortion is due to extra loading dye in these lanes to compensate for their tendency to float away (residual ethanol contamination). This gel is one of two identical, independent experiments. These results are representative of those observed in more than 5 similar experiments.

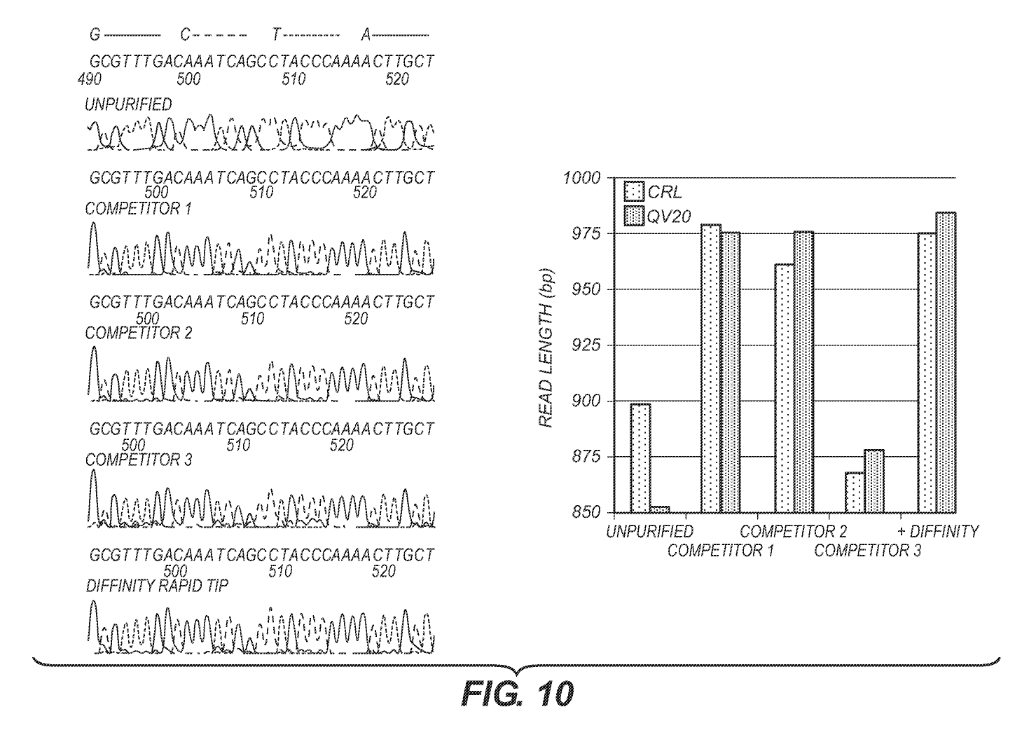

FIG. 10 shows Sanger sequencing is a quantitative functional test of purified PCR solutions. Unpurified simulated PCR solution was purified with several competitors' products (Q, I, and E) as well as with the Diffinity tip and spin column according to manufacturer's protocols or as described in Methods. Left: Two quantitative measures of sequence quality, the continuous read length (CRL) and quality value (QV20) are plotted above with each column representing the average of two identical, independent sequence reactions. Longer, higher quality read lengths have higher values while unpurified sample acts as a negative control and has lower values. Right: Sequence chromatograms from the middle region of the available sequence visually show an improvement in peak quality between unpurified and most purified samples as exhibited with an improved signal-to-noise ratio. These results are representative of those observed in more than 5 similar experiments.

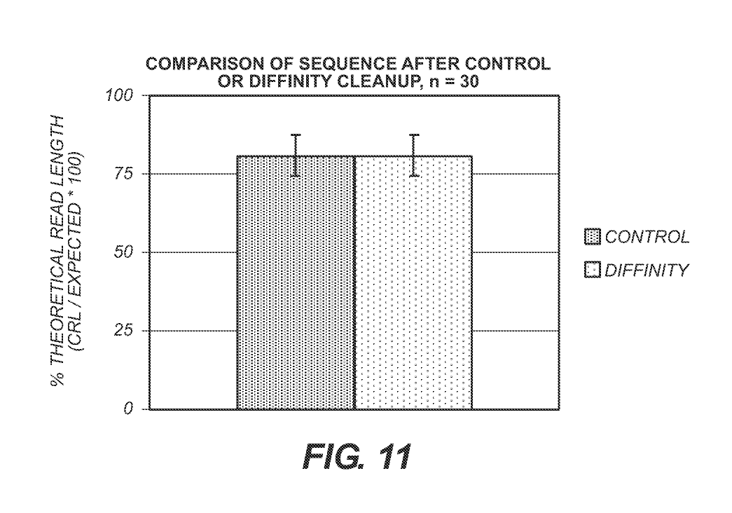

FIG. 11 is a comparison of sequences after cleanup with a control purification system and an embodiment of the fluid sample purification system (“Diffinity functional PCR purification tip,” “Diffinity tip”). Independently run beta tests were conducted at 5 sites to compare the Diffinity functional PCR purification tip to methods currently used by the respective testing labs. PCR samples were divided to be used with either control (80.92+/−6.59%) or Diffinity (80.99+/−6.51%) PCR Purification. After sequencing, the continuous read length (CRL) was normalized as a percentage of the expected read length and averaged for each group. Shown is the average of 30 trials with standard error bars.

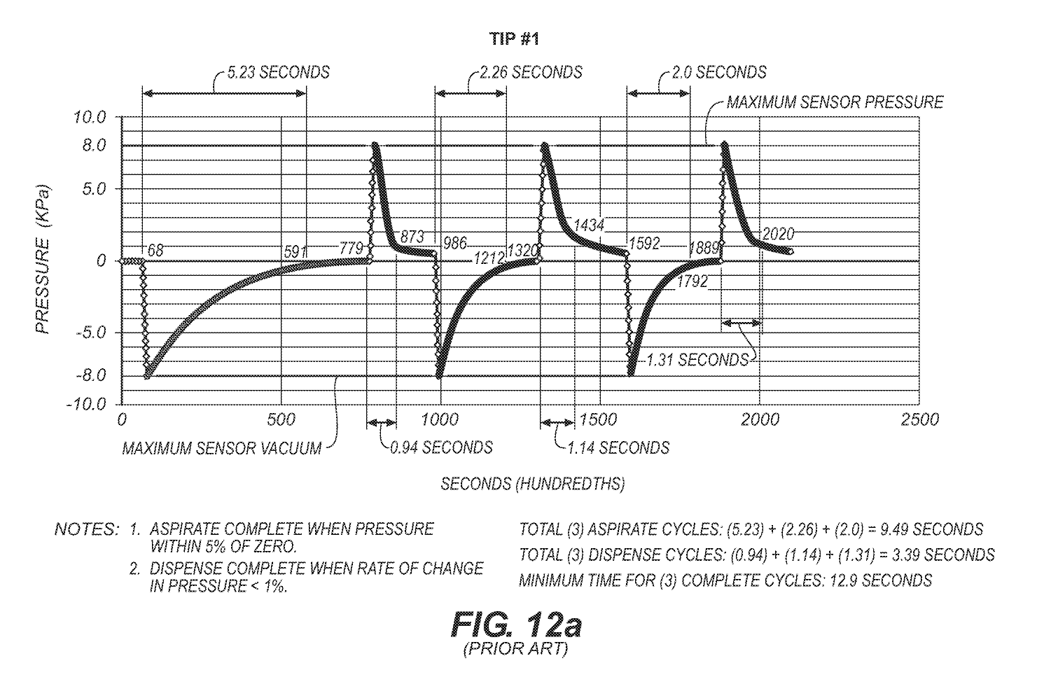

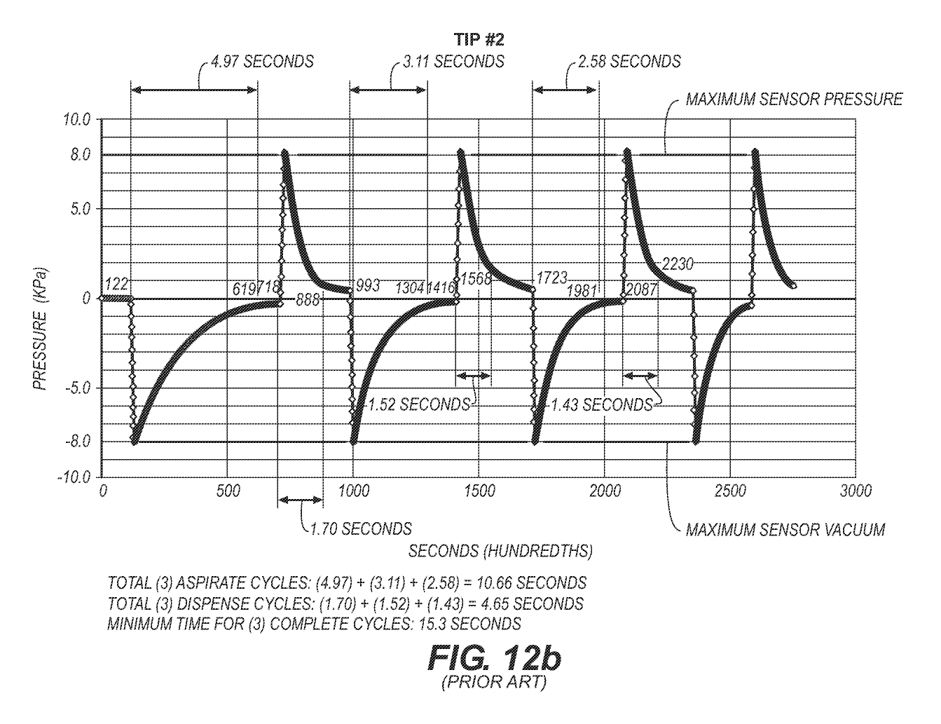

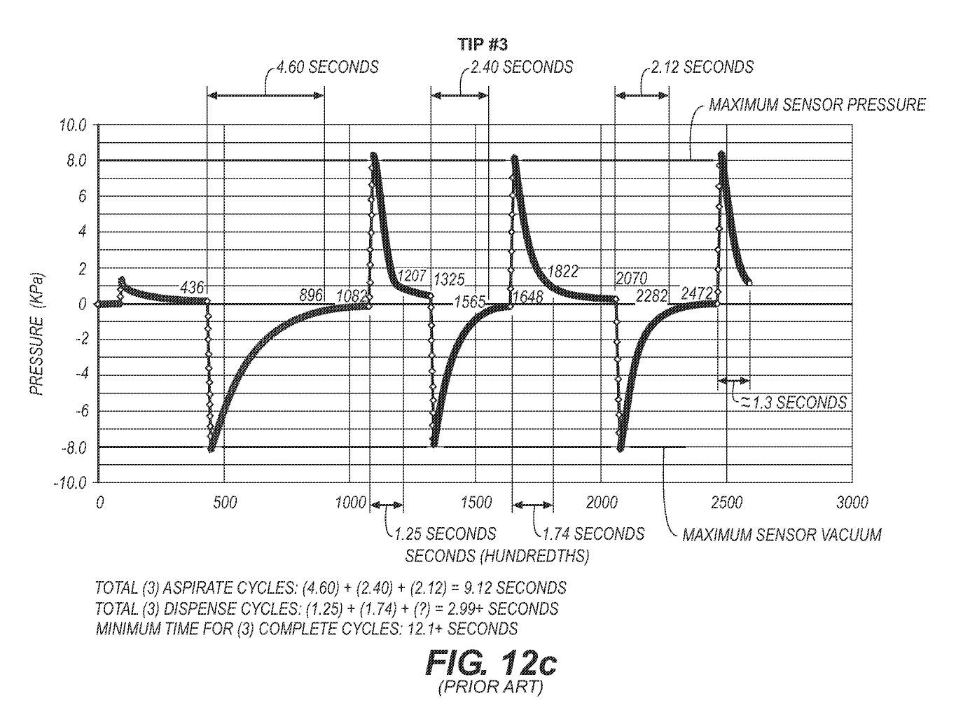

FIGS. 12a-c shows typical sample aspirate and dispense pressures within a commercially available Thermo Fisher Scientific Aspire™ pipette tip (“Prior Art Tip”) as it aspirates and dispenses water. As shown in FIGS. 12a-c, a typical minimum total cycle time for three successive sample aspirate and dispense cycles is approximately 13.4 seconds. The average sample aspiration cycle time (first three aspiration cycles) is approximately 3.3 seconds per aspiration. The average sample aspiration cycle time includes a average first aspiration cycle time of approximately 4.9 seconds

Removing the first aspiration cycle time from subsequent aspiration cycles provides an average cycle time of approximately 2.4 seconds each for aspiration cycles #2 and #3. Average sample dispense cycle time for three successive sample dispense cycles is approximately 1.3 seconds per dispense.

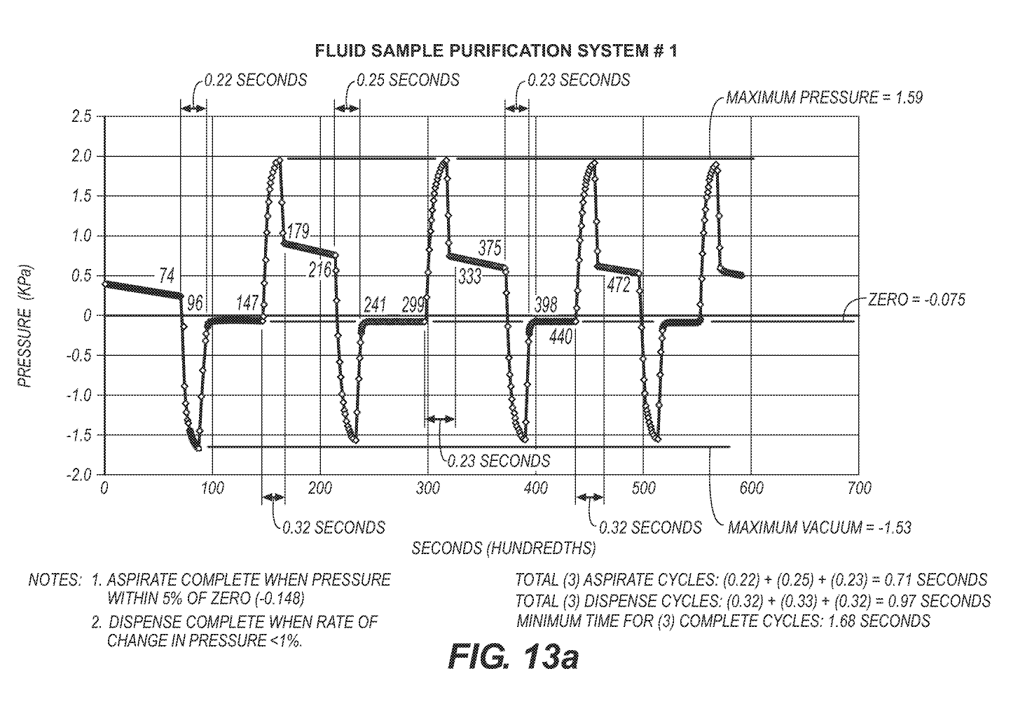

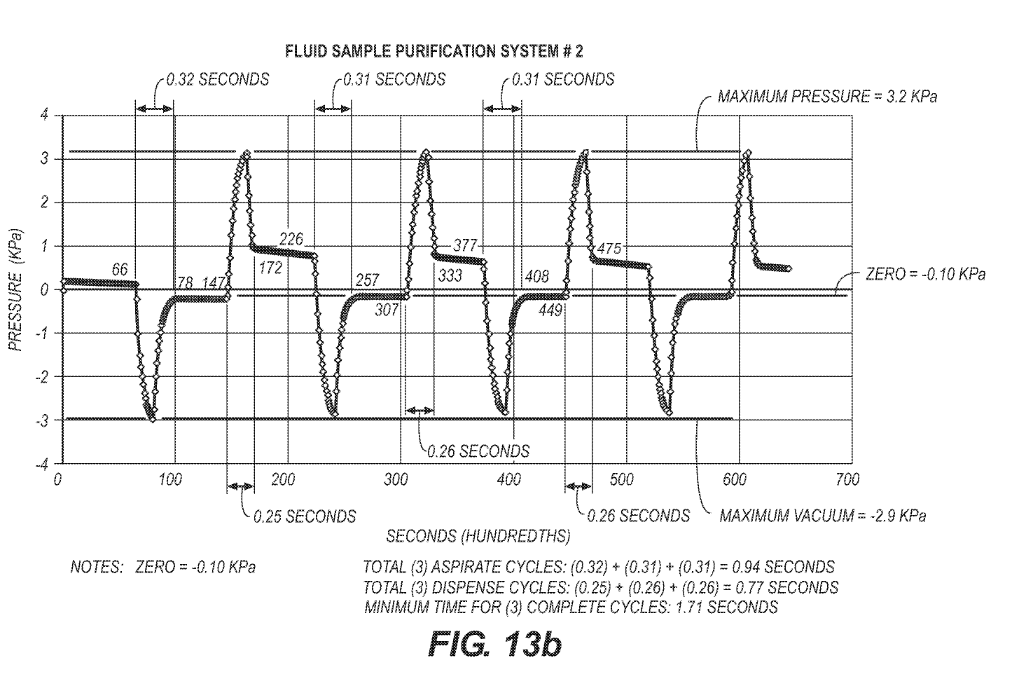

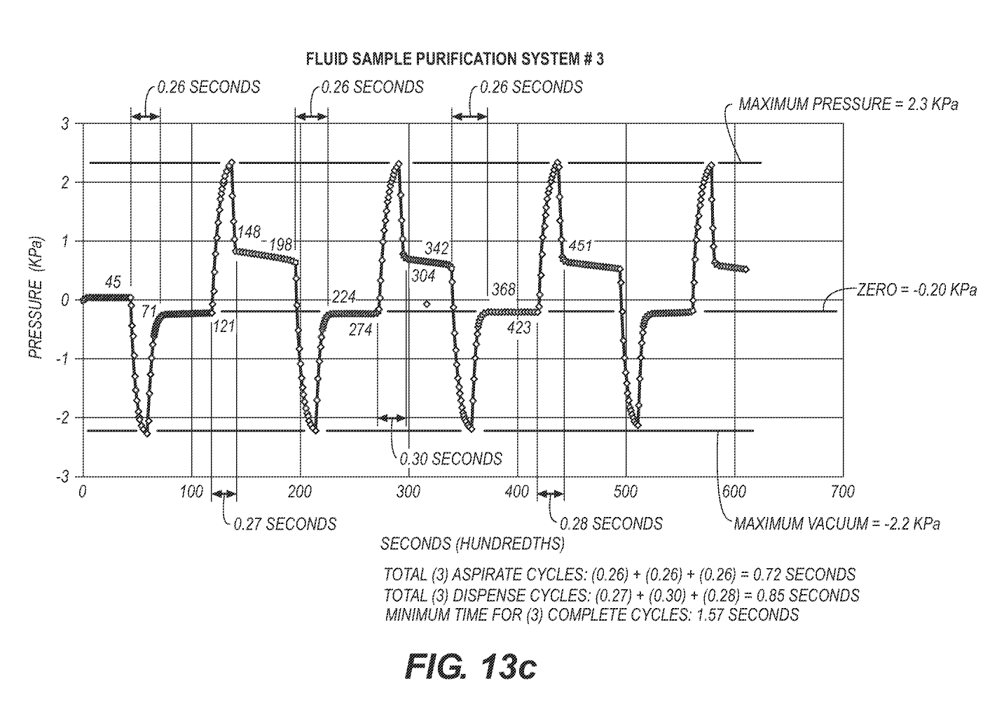

FIGS. 13a-c shows typical sample aspirate and dispense pressures within a pipette tip of the fluid sample purification system as it aspirates and dispenses water. A typical minimum total cycle time for three successive sample aspirate and dispense cycles is approximately 1.7 seconds, which is approximately 87% less than that of the Aspire™ product. Average sample aspiration cycle time (first three aspiration cycles) is approximately 0.26 seconds per aspiration, which is approximately 92% less than that of the Aspire™ product.

The average time for the first sample aspiration cycle is approximately 0.27 seconds, essentially identical to the average sample aspiration cycle time of 0.27 seconds for the second and third aspiration cycles, indicating the absence of any first aspiration effect (e.g., wetting of adsorption media). The average first aspiration cycle time of approximately 0.27 seconds is approximately 91% less than that of the Aspire™ product.

Average sample dispense cycle time (first three dispense cycles) is approximately 0.29 seconds per dispense, which is approximately 77% less than that of the Aspire™ product.

The fluid sample purification system is able to achieve these improvements in part because it has fewer filters. In one embodiment, the fluid sample purification system uses a single filter (or frit) to retain adsorption materials (e.g., particles) at the distal end of the pipette tip prior to use.

In another embodiment, adsorption material is retained at both the proximal and distal ends by a frit, filter or retainer.

In another embodiment, adsorption material is retained at the proximal end by a pierceable membrane that is removed (i.e., pierced) at the moment of use. Adsorption material can be retained during use by a portion (e.g., the tip) of the pipettor, which pierces the pierceable membrane. The use of a single filter can therefore provide inherently less resistance to fluid flow. The increased rate of fluid flow associated with this aspect of the fluid sample purification system helps provide the following specific benefits:

(1) Reduced purification process time owing to faster fluid aspiration and dispense cycle times.

(2) Reduced purification process time owing to the faster adsorption time as a result of the greater level of agitation caused by the higher fluid flow rates during sample aspiration and dispensing.

(3) Reduced product manufacturing cost by eliminating a potentially expensive component.

(4) Improved product reliability by eliminating a critical component from the product design.

Although not directly related to fluid flow rate, the elimination of a second, fluid transmitting filter also reduces the potential to adsorb desired target (e.g., ds-DNA) which decreases target yield.

### 5.2 Housing

The housing can he in any pipette tip or column format known in the art.

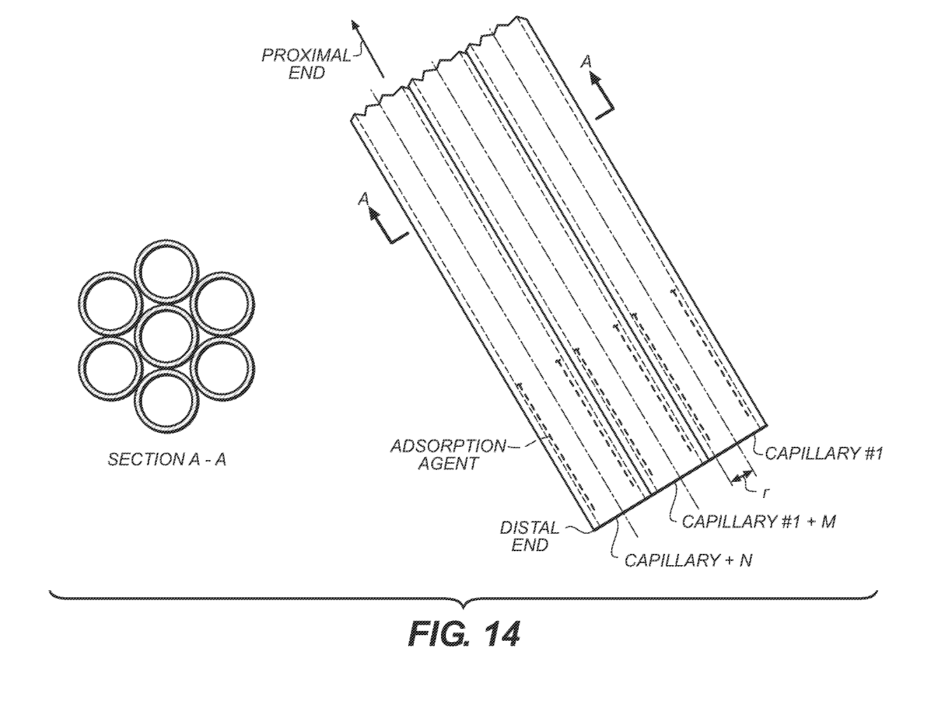

In one embodiment, the housing can be one or more capillary tubes (FIG. 14). In this embodiment, sample fluid is aspirated and dispensed i) from within the capillary tube(s), or ii) around the external surfaces the capillary tube(s) which form capillary ducts, or iii) both the internal and external surfaces of the capillary tube(s). In this embodiment the adsorption material is placed on i) the internal surfaces of the capillary tube(s), or ii) the external surfaces of the capillary tube(s), or iii) both the internal and external surfaces of the capillary tube(s).

This embodiment has the further advantage of creating a large coated surface area to fluid volume ratio. It also has the advantage of using a small housing (capillary) radius (“r”) to minimize mass transport diffusion path length and diffusion time so as to minimize the time required for materials in the aspirated sample to reach the surface of the capillary tube(s) and be adsorbed. It also allows the use of capillary pressure alone to aspirate the sample, thereby eliminating the need for additional instrumentation to aspirate fluid. It also provides the ability to purify very small volumes of sample, such as sample volumes less than 10 μl.

In this embodiment, the purified fluid is forced from the capillary tube(s) by such means as i) application of positive fluidic pressure, ii) centrifugation, or iii) a greater capillary force caused by, for example, bringing the fluid-bearing capillary into contact with a hydrophilic material having greater surface energy (i.e., greater capillary pressure) sufficient to draw the fluid from the capillary tube(s).

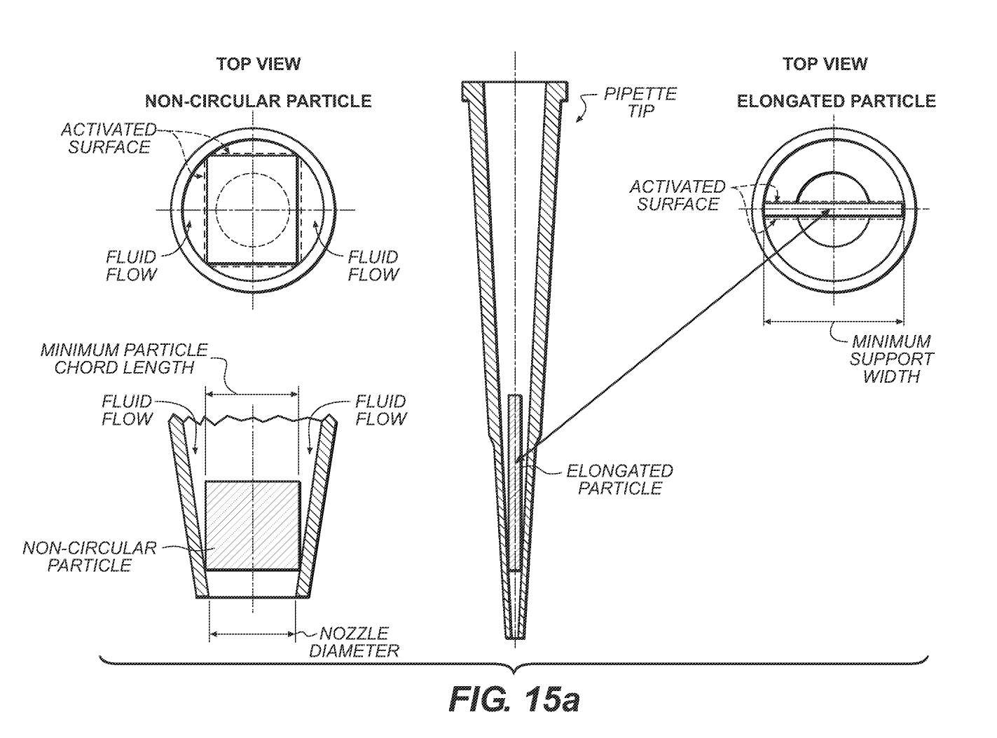

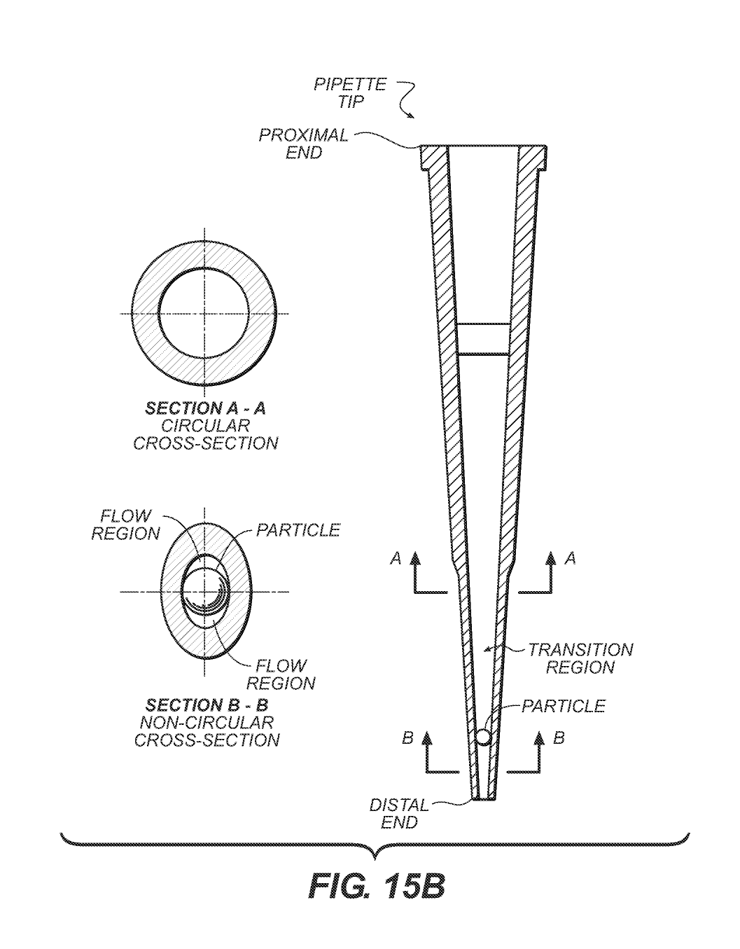

In another embodiment, the housing can be a pipette tip that comprises a non-circular or non-spherical pipette tip nozzle orifice. This housing can be used to contain spherical or near-spherical shaped particles, wherein the non-spherical nature of the nozzle simultaneously retains the spherical particles within the pipette tip while also preventing the particles from plugging of the pipette tip. In this particular embodiment, the particles are contained as described above, wherein the minimum chord length of the particle is greater than the maximum dimension across the non-circular pipette tip nozzle and/or the maximum effective size of the openings of the retaining means at either the distal and/or proximal ends of the pipette tip. This aspect is shown in FIG. 15b, where a spherical particle is retained by an elliptically shaped pipette nozzle. In this particular embodiment, the minimum diameter of the particle is greater than the maximum length of the minor axis of the elliptically shaped nozzle.

### 5.3 Functionalized Particles and Other Adsorption Materials

The fluid sample purification system and methods can employ, in certain embodiments, adsorption materials as described in U.S. provisional application No. 61/151,551, filed Feb. 11, 2009, entitled “Spatially Inhomogenously Functionalized Porous Media and Method for Use in Selective Removal of Contaminants” by Lewis J. Rothberg and in International Application No. PCT/US10/23900, filed Feb. 11, 2010, entitled Spatially Inhomogenously Functionalized Porous Media and Method for Use in Selective Removal of Contaminants (Applicant: University of Rochester), and summarized as follows.

The fluid sample purification system can use adsorption materials comprised of nanoporous materials in which the internal surfaces (pore surfaces) are functionalized to adsorb small molecules (e.g., primers and dNTPs) and the external surfaces are functionalized to inhibit adsorption of larger molecules (e.g., double stranded PCR amplicons). Since the larger ds-DNA is excluded from the interior of the pores in this example, only the small molecules will be adsorbed to the nanoporous materials and thus, according to this embodiment, double stranded DNA can he purified from the mixtures due to the differential functionalization of interior and exterior surfaces.

Functionalized particles that can used in the fluid purification system embodiments can comprise i) a single type of particle containing one specific impurity adsorption and target rejection chemistry, ii) a single type of particle containing more than one impurity adsorption and/or target rejection chemistry, iii) multiple types of particles each containing one impurity adsorption and target rejection chemistry, and iv) multiple types of particles each containing more than one impurity adsorption and/or target rejection chemistry. Likewise, any of the adsorbing/rejection surfaces used in the fluid purification system embodiments can have one or more regions within the surface configured with one or more of the above adsorption/rejection chemistry configurations.

In a specific embodiment, functionalized particles of a plurality are differentially functionalized particles, i.e., the plurality can comprise two or more types of functionalized particles.

In identifying suitable nanoporous materials, the size of the small molecules which are desired to be removed (e.g., primer-dimers and unincorporated primers and dNTPs) is taken into consideration. These are typically a few nanometers (nm) in size. Therefore, nanoporous materials having pore sizes of approximately 5 nm to approximately 150 nm will allow adequate access for the contaminants into the pores. Such pores will also exclude larger molecules such as double stranded DNA amplicons that can be several hundred or more base pairs in length. In some embodiments, not all the pores in the nanoporous materials will be of the same size. However, in various embodiments, preferably, 70, 80, 90 or 95% fall within the range of 5 to 150 nm.

The nanoporous material may be configured as a surface, particulate or in the form of a foam, or gel. Suitable nanoporous particulate materials can include, e.g., porous silica, porous alumina, and zeolites. Suitable high surface area gels can include silica, alumina, zeolites and titania. In one embodiment, chromatography grade nanoporous silica is used, which can be obtained as a substantially spherical particle with a diameter of 40-60 microns, and a pore size of approximately 60 Å. A material with these (commercially available, e.g., Merck 9385 silica gel).

Functionalized particles can be used as adsorption materials in the fluid sample purification system. The chemical and biochemical characteristics of suitable functionalized particles to be employed in embodiments of the fluid sample purification system and methods are described in U.S. provisional application No. 61/151,551, filed Feb. 11, 2009, entitled “Spatially Inhomogenously Functionalized Porous Media and Method for Use in Selective Removal of Contaminants” by Lewis J. Rothberg and in International Application No. PCT/US10/23900, filed Feb. 11, 2010, entitled Spatially Inhomogenously Functionalized Porous Media and Method for Use in Selective Removal of Contaminants (Applicant: University of Rochester).

In addition, the particles have certain physical size and particle type characteristics. The total surface area of a given volume of particles increases with decreasing particle size. Increased total surface area in the fluid sample purification system provides the advantage of being able to contain a greater quantity of adsorption material, since the amount of adsorption material is dependent upon available surface area. However, decreasing particle size also makes it harder to remove fluid from a wetted mass of particles by increasing the capillary pressure or “unwetting pressure” of fluid at the fluid-air interface. It is known in the art that capillary pressure is defined as P_{c}=2γ/r where γ is the surface tension of the fluid and r is the radius of the fluid surface at the fluid-air interface, which decreases with decreasing particle size. Recovering a high percentage of sample fluid from a sample treatment process is often very important, particularly when small volumes of sample are available.

In another refinement of the present invention, the size of the particles for supporting the adsorption material is between 1.0 and 1000 μm. The choice of specific particle size is determined to a large extent by the requirements of the specific application and specific product embodiment used. In one embodiment, particle sizes of 1.0 to 10 microns are used to increase the surface area of the adsorption materials. Due to the very small between-particle pore size, the use of particles of these sizes requires special measures to remove fluid from the particles, such as centrifugation which is acceptable in some applications. Particle sizes of 10 to 150 microns provides suitable surface area for end-use applications such as purification of PCR solutions, where the mass of impurities to be removed (e.g., dNTPs and primers) is consistent with the adsorption material that can be placed on the available particle surface area, while at the same time providing unwetting pressures that enable fluid to be removed relatively easily, such as with the application of a slight pressure from a fluid pipettor, which is typically 5-25 Kilo-Pascals. Particle sizes of 100 to 1000 microns provide lower particle surface area that is suitable for end-use applications where the mass of impurities to be removed is relatively low and consistent with the adsorption material that can be placed on the available particle surface area, while at the same time providing very low unwetting pressures that enable fluid to be removed very easily, such as the use of gravity to drain the fluid from the particles. This size of particle is also much easier to retain in a pipette tip such as, for example, using integral pipette tip particle retention ribs (FIG. 5b and FIG. 26).

This aspect includes the advantages of maximizing the surface area of the adsorption material. It simultaneously minimizes the pressure required to force liquid from a mass of wetted particles by using choice of particle size to lower the pressure required for efficient removal of fluid, consistent with the fluid handling instrument and methods available in the intended application.

Further regarding the physical size of the particles, the particles are preferably small enough to avoid rapid sedimentation within the fluid sample, since sedimentation prevents them from interacting with any impurities that may be in the sample solution. Determining such a suitably small particle size is known in the art.

In one embodiment, a range of particle sizes is used. In a specific embodiment, the particle size can range from 10-100 μm, and preferably 50-100 μm. The majority of the particles remain uniformly suspended in solution (FIG. 16) while the sample solution is contained within the fluid sample purification system, such as the pipette tip-based embodiment shown in FIG. 6a.

Depending upon the type of pipettor used (e.g., manual or automated) and operator technique (e.g., fast or slow), the residence time of the sample solution within the pipette tip, or the time between sample aspirate and dispense cycles, is typically 1 to 5 seconds. Thus a majority of the particles preferably remain suspended throughout the bulk of the sample solution during this time for effective interaction with any impurities that may be in the solution. The uniform suspension of the particles helps to create a shorter mean diffusion path length between any impurities that may be in the sample solution and the impurity-adsorbing particles, thereby minimizing the time required to remove (i.e., adsorb) the impurities and thereby purify the sample solution.

FIG. 16 shows calculations for predicted settling times using methods described by Bird, Stewart and Lightfoot (1960, Transport Phenomena, John Wiley & Sons, ISBN 047107392X). Settling times can also be determined experimentally by observing how quickly a turbid solution of larger particles can clear.

As shown in FIG. 16, for a within-tip sample height of 1.5 cm, 50% of particles larger than approximately 100 μm sediment out of solution (i.e., travel one-half the sample height) in less than approximately 1.1 seconds. Likewise, for within-tip sample heights of 1.0 and 0.5 cm, 50% of particles larger than 90 μm and 60 μm sediment out of solution in less than approximately 0.9 and 1.1 seconds, respectively. Since these particles are no longer available to adsorb impurities from the entire bulk of the sample solution, particles larger than 60 to 100 μm represents non-optimal design values, depending upon actual sample volume, pipette tip design and subsequent height of the sample column within the pipette tip. At the same time, while particles less than approximately 10 μm have very long sedimentation times and therefore sustained interaction with solution impurities, they can easily become airborne and are undesirable from a product manufacturing and health perspective. This then creates an optimum particle design size of greater than 10 μm and less than 60 to 100 μm, again depending upon actual sample volume, pipette tip design and subsequent height of sample column within the pipette tip.

According to this embodiment, the particles are preferably of a suitable size large enough to enable the use of filters, particularly a distally-positioned filter, with the larger pore sizes required to enable high rates of fluid sample flow into and out of the pipette tip while not passing the particles—a conflicting requirement. General methods for determining suitable particle sizes are known in the art.

The particles can be spherical (or approximately spherical) or can be non-spherical.

The non-spherical nature of the particles can, in certain embodiments, prevent plugging of a typically circular pipette tip nozzle (FIG. 15a). For example, particles can be faceted solid particles (e.g., cubical or tetrahedral shaped particles). In this particular embodiment, the particles are contained as described herein, wherein the minimum chord length of the particle (e.g., the length of the shortest side of the rectangular particle shown in FIG. 15a) is greater than the pipette tip nozzle diameter and/or the maximum effective size of the openings of the distal and proximal particle retaining means within the pipette tip.

High fluid flow rates are preferable since they help to minimize overall sample processing time in several ways, including the following. First, high fluid flow rates minimize the time required to move a given volume of fluid sample into and out of the pipette tip with each aspirate and dispense cycle. Second, high fluid flow rates create higher agitation of the fluid within the pipette tip, thereby bringing the adsorption particles into more frequent contact with the suspended impurities.

The particles are preferably of a large enough size to enable the fluid retained with their void volume (i.e., the volume of air or liquid between adjacent particles) to be easily recovered. This can be determined for example, by creating a large capillary radius, which reduces the capillary pressure, which in turn reduces the pressure to force liquid through and out of the porous media. Another approach is to increase the wettability of the porous media, which also reduces the capillary pressure with the same effect. Further discussion and data supporting the effect of high fluid flow rate upon sample purification time and a more detailed discussion of fluid sample recovery are described hereinbelow.

Preferable particle sizes for the purification pipette tip are 50-100 μm, for the following reasons:

(a) Rapid purification time owing to the elimination of significant particle sedimentation during sample handling, because the fluid sample purification system uses smaller particle sizes (i.e., 100 μm or less).

(b) Rapid purification time owing to higher fluid flow rates and greater agitation associated with the larger pore size filters because the fluid sample purification system uses larger particles (i.e., greater than 50 μm).

(c) Improved sample quality owing to the ability of smaller particles to more effectively remove impurities within the sample solution (e.g., minimal sedimentation).

(d) Improved sample recovery owing to the ability to more easily recover retained fluid from larger particles.

(e) Easier and safer product manufacturing owing to the use of larger particles (i.e., greater than 10 μm).

(f) Optimum particle size for achieving shorter purification times, higher target yield, higher sample recovery and ease and safety of product manufacturing.

The fluid sample purification system can comprise a reaction chamber, which is described below in Section 5.16. In one aspect, the reaction chamber uses adsorption particles that have specific gravity greater than 1.0, which eliminates the need to confine the particles (e.g., the void volume disclosed in WO88/09201 A1 by Colpan et al.) and as a result, improves system performance.

The amount of undesirable material that can be removed from a solution and the volume of adsorption material required to remove the undesirable materials are important aspects of any fluid sample purification system design. Since smaller particles create higher surface areas for adsorbing greater quantities of undesirable materials per unit volume of adsorption material, smaller particle size is desirable.

In another embodiment, porous particles are used for adsorption, as porous particles can provide a much greater area per unit volume for any given size of particle.

As discussed above, recovery of a high percentage of sample volume is yet another important system performance parameter, particularly when working with small sample volumes. However, smaller particles can aggregate together and retain liquid within the voids between adjacent particles. Wetted particles create a capillary pressure at the fluid-air interface, which is defined as P_{c}=2γ/r where γ is the surface tension of the fluid and r is the radius of the fluid surface at the fluid-air interface. For a 25 or 50 μm capillary radius of water, the capillary pressure would be approximately 9.0 and 4.5 pounds per square inch, respectively, much more than the approximately 1.0 pound per square inch developed by conventional pipettors, making it very difficult for a pipettor to force liquid out of a wetted, aggregated mass of particles of this size. Therefore from this perspective, larger particle size is preferred. However, even at the maximum optimal particle size of 100 μm previously identified, unwetting pressures will be approximately 2.3 pounds per square inch, still higher than available pipettor pressures and likely to adversely affect sample recovery.

All else being equal, a smaller mass of smaller particles helps to minimize the volume of liquid retained in the voids between adjacent particles. However, a smaller mass can possibly adversely affect particle surface area available for adsorbing undesirable materials.

In other embodiments, methods for overcoming these competing parameters are provided: through the use of hydrophilic particles and porous particles, which either by themselves or together simplify sample volume recovery while providing other beneficial performance benefits.

First, the hydrophilic nature of the particles serves to make the particles more wettable (e.g., reducing advancing and receding contact angle between the fluid and the surface of the solid particle) thereby increasing the capillary radius and subsequently reducing the unwetting pressure to levels achievable with conventional manual or automatic pipettors.

Second, the porous particles provide a very high particle surface area to volume ratio, relative to the same sized solid particles, thereby enabling a smaller mass of particles to be used to adsorb an equivalent level of impurities, while at the same time trapping less sample volume owing to their smaller total volume. An example of a typical porous particulate material is chromatography grade nano-porous silica, which can, for example, be obtained as a substantially spherical particle with a diameter of 40-60 microns, and a pore size of approximately 60 Å. A material with these characteristics is commercially available, such as Merck 9385 silica gel. A 2 mg quantity of particles carries ˜10,000 cm² surface area. By comparison, 2 mg of 50 micron solid silica particles would provide a surface area of ≈550 cm².

Third, the greater adsorption capacity of the lower volume of porous particles helps to ensure a high level of sample quality over a wider range of impurity concentrations within a fluid sample. This feature is particularly important, for example, when purifying PCR solutions, where failure of the PCR solution to amplify the desired DNA target would leave very high levels of unincorporated nucleotides and primers in solution, relative to a successful PCR process, thereby making recovery of the (unamplified) target DNA difficult to achieve.

Fourth, by reducing the volume of particles required to remove a specific mass of impurities, porous particles help to maintain a high rate of fluid flow through the (distal) filter(s) used to retain them within a pipette tip. More specifically, particle retention filters are generally known as surface filters, meaning that retained particles build up on their upstream surface owing to the larger size of the particle relative to the effective pore size of the filter.

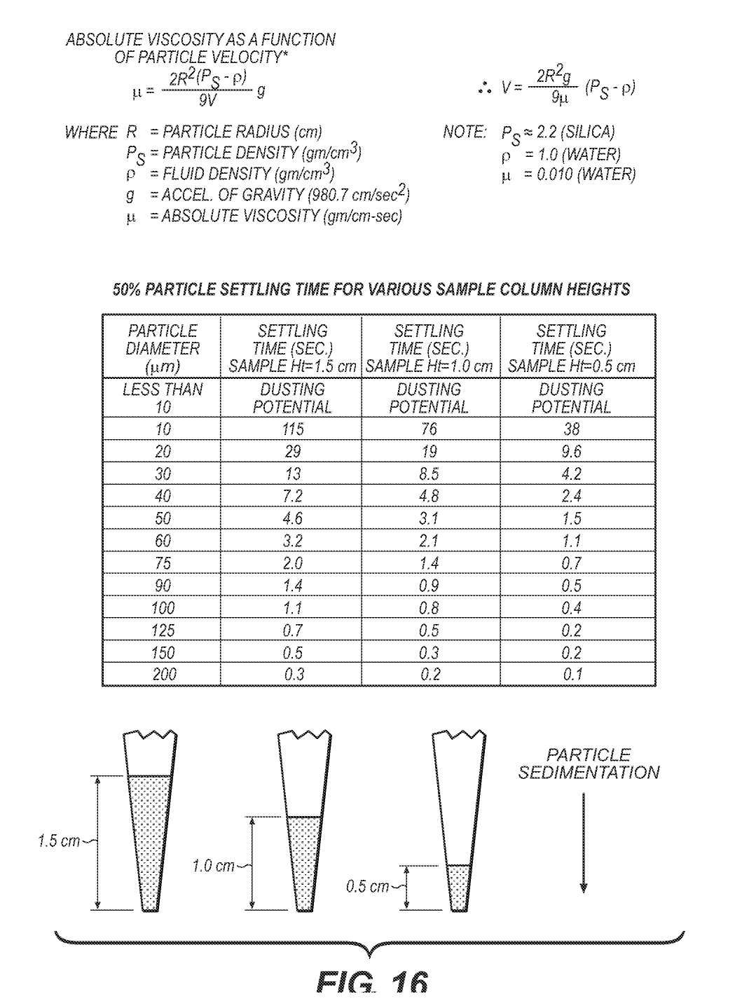

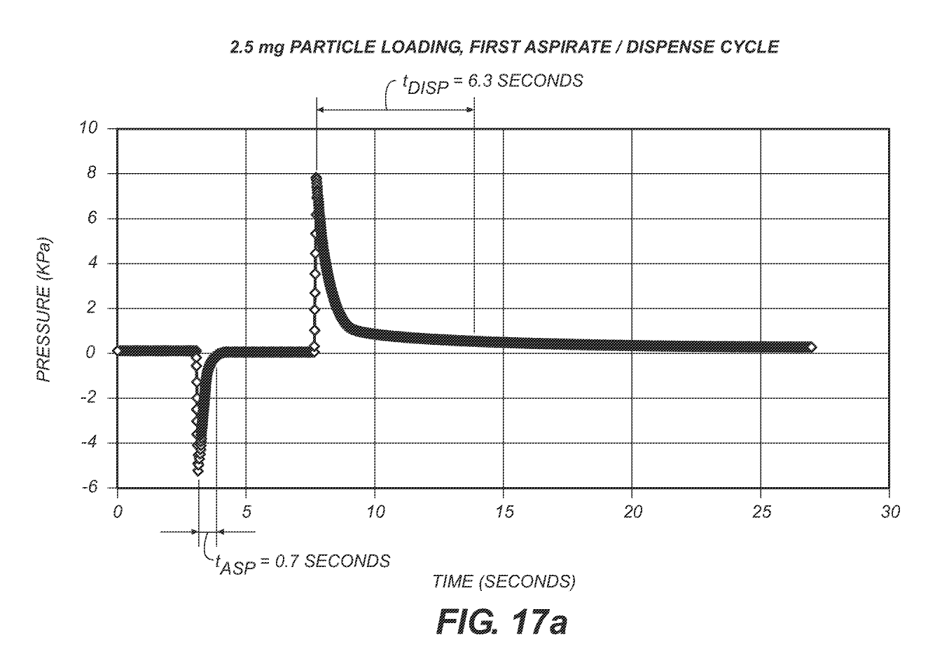

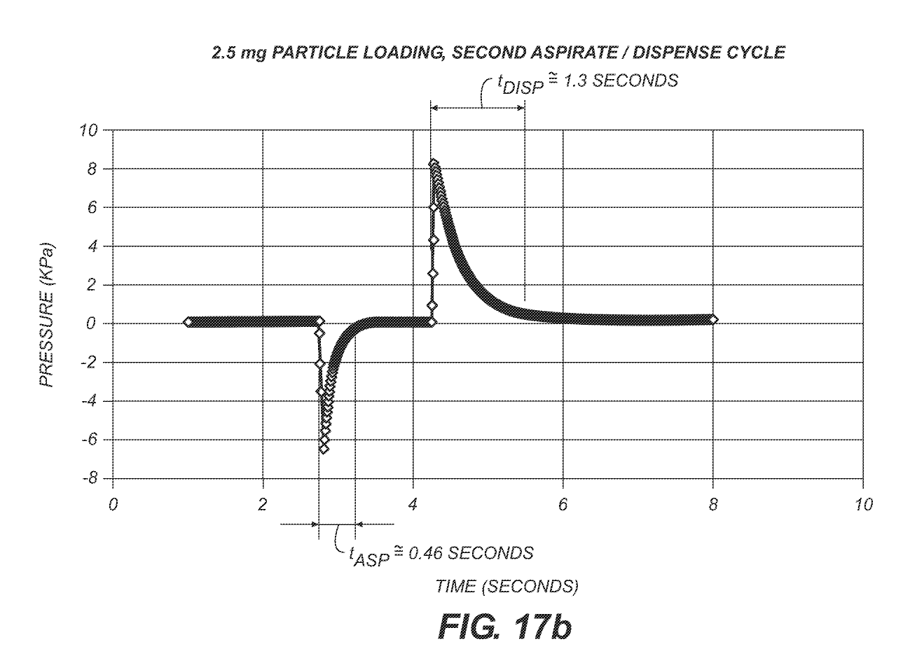

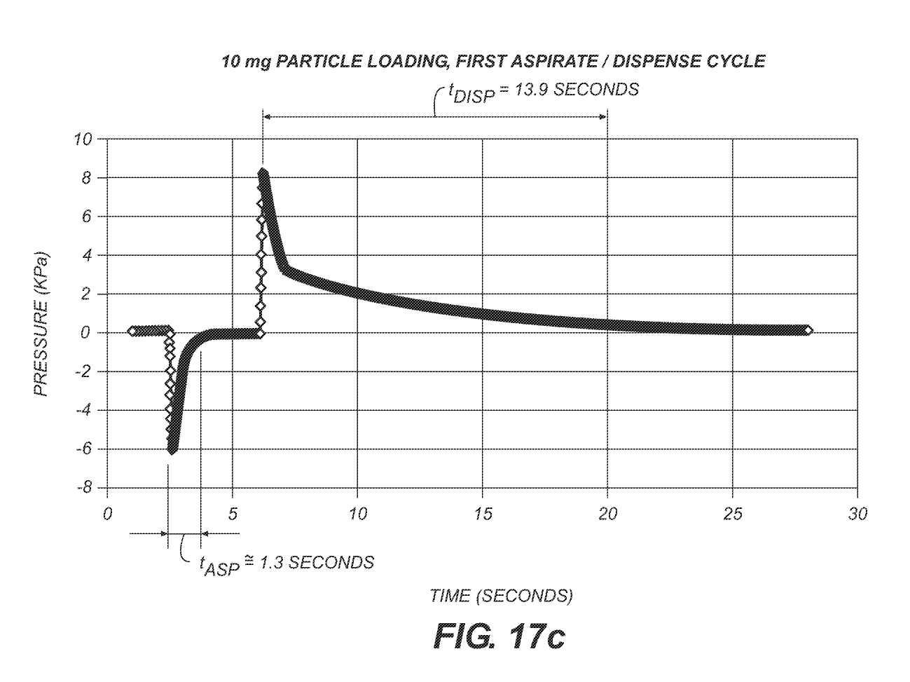

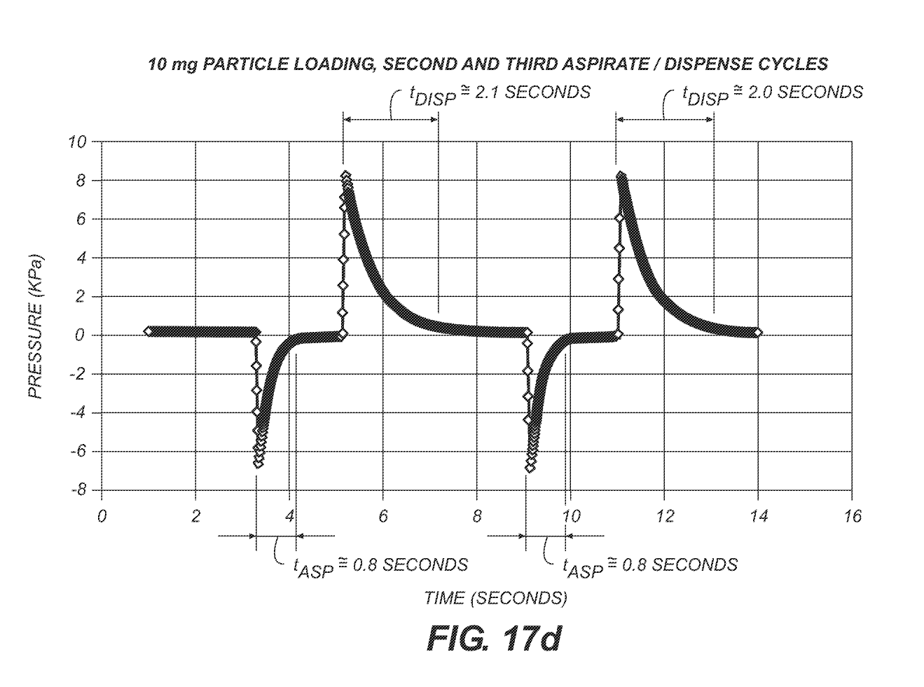

FIGS. 17a-d show results from pipette tips that were loaded with either 2.5 or 10.0 milligrams of unfunctionalized particles with a hydrophobic surface. Fluid was then aspirated and dispensed from the tips.

FIG. 17a shows that tips containing 2.5 mg of particles had first fluid aspirate and dispense cycle times of 0.7 and 6.3 seconds, respectively.

FIG. 17c shows that tips containing 10.0 mg of particles had first fluid aspirate and dispense times of 1.3 and 13.9 seconds, respectively, reflecting an approximately 2× increase in first fluid aspiration and dispense cycle times, relative to the tips containing 2.5 mg of particles.

FIG. 17b shows that tips containing 2.5 mg of particles had subsequent fluid aspirate and dispense times of 0.5 and 1.3 seconds, respectively.

FIG. 17d shows that tips containing 10.0 mg of particles had subsequent fluid aspirate and dispense times of 0.8 and 2.0 seconds, respectively, reflecting an approximately 60% increase in subsequent fluid aspiration and dispense cycle times, relative to the tips containing 2.5 mg of particles.

These data show that larger loading of particles within the pipette tip serve to increase fluid aspirate and dispense times for first and subsequent aspiration and dispense cycles.

FIGS. 13a-c shows that first aspirate cycle times were 0.22, 0.32 and 0.26 seconds for three pipette tips of the fluid sample purification system, or an average of 0.27 seconds. Second and third aspiration times averaged 0.27 seconds. These pipette tips contained 2.5 mg of hydrophilic particles.

The overall (i.e., first and subsequent cycles) average aspirate time of 0.27 seconds is approximately 38% of the first aspirate cycle time of 0.7 seconds associated with the pipette tips with 2.5 mg of hydrophobic particles, which indicates that the use of hydrophilic particles results in much shorter first aspiration cycle times, relative to the use of the same amount of hydrophobic particles under similar conditions.

When hydrophilic particles are used, there is also no difference in first and subsequent fluid aspiration cycle times; all are equally fast, relative to current pipette tips available in the art (see FIGS. 12a-c), and the case in the fluid sample purification system, where hydrophilic particles are used.

FIGS. 13a-c show first dispense cycle times of 0.32, 0.25 and 0.27 seconds for three pipette tips of the fluid sample purification system, or an average of 0.28 seconds. The second and third aspiration times average 0.29 seconds. These pipette tips contained 2.5 mg of hydrophilic particles.

The overall (i.e., first and subsequent cycles) average dispense time of 0.29 seconds is approximately 5% of the first dispense cycle time of 6.3 seconds associated with the pipette tips with 2.5 mg of hydrophobic particles, which indicates that the use of hydrophilic particles results in much shorter first aspiration cycle times, relative to the use of the same amount of hydrophobic particles under similar conditions.

When hydrophilic particles are used, there is also no difference in first and subsequent fluid dispense cycle times; all are equally fast, relative to currently (see FIGS. 12a-c) and the case in the fluid sample purification system, where hydrophilic particles are used.

The faster fluid aspirate and dispense cycle times of the fluid sample purification system, relative to the current art (e.g., FIGS. 12a-c) are likely owing to the use of a smaller mass of particles (made possible by the use of porous particles), the use of hydrophilic particles, and the use of fewer and more efficient filter(s). The dramatic difference between typical art-known first fluid dispense times (see FIGS. 12a-c) and those using the system and methods of the fluid sample purification system and method reflect the particular advantage provided by the use of hydrophilic particles, fewer particles, fewer filters and filters with less resistance to fluid flow.

Typical solid phase extraction columns require a longer “wetting” phase. This phenomenon is present when current design functional pipette tips are used (see FIGS. 12a-c). The fluid sample purification system and method overcome this problem by the use of hydrophilic particles, and by the use of smaller masses of particles for any given extraction capacity requirement, owing to their porous nature.

The use of hydrophilic particles in the fluid sample purification system and methods also helps to reduce sample processing (e.g., sample purification) time by eliminating the time required to wet the adsorption particles and initiate the chemical aspect of the sample purification process. Finally, the use of hydrophilic particles also reduces the potential to entrap air within the particles during initial wetting, which helps to improve reliability of operation and sample processing cycle time (i.e., the time required to urge the entrapped air bubble).

Surface wetting is a classical problem with existing design chromatography columns. FIGS. 12a-c show that an excessively long first sample aspiration time is associated with the Aspire™ design functional pipette tip.

Particles can be designed for enabling rapid removal of a high level of impurities while minimizing retained fluid sample volume, while simultaneously satisfying all other previously discussed particle design constraints.

The functionalized particles are porous, thus providing a larger adsorption surface area with the ability to effectively scavenge a larger mass of impurities while simultaneously small enough to minimize the volume of retained fluid sample while still large enough to minimize filter or retainer clogging. In one embodiment, the porous particles significantly increase (i.e., 10-20×) adsorption capacity.

In one aspect, the porous particles are located within the pipette tip of the fluid sample purification system. The very high surface area to volume ratio of these particles, approximately 10-20× greater than non-porous particles, enables an equivalent 10-20× reduction in the volume of adsorption particles used within the pipette tip, for a given adsorption capacity requirement. Since the accumulation of particles within and/or against the face of the filter increase resistance to flow, this feature of the fluid sample purification system helps to increase fluid sample flow rates, which in turn help to reduce purification time, owing to shorter sample aspiration and dispense times and the higher agitation caused by the higher flow rates. Evidence of these phenomena is seen in FIGS. 17a-d.

FIGS. 12a-c and 13a-c show that the Aspire™ pipette tip has maximum aspirate and dispense pressures in excess of 8.0 KPa (i.e., maximum response of the pressure sensor used for making these measurements) versus the fluid sample purification systems average aspirate and dispense pressures of 2.2 and 2.4 KPa, respectively, or 75% less than those of the Aspire™ values. Since fluid flow rate is generally linear with pressure, this means that for any given pipettor pressure, the fluid sample purification system will achieve higher fluid sample flow rates and the many benefits that these higher flow rates provide.

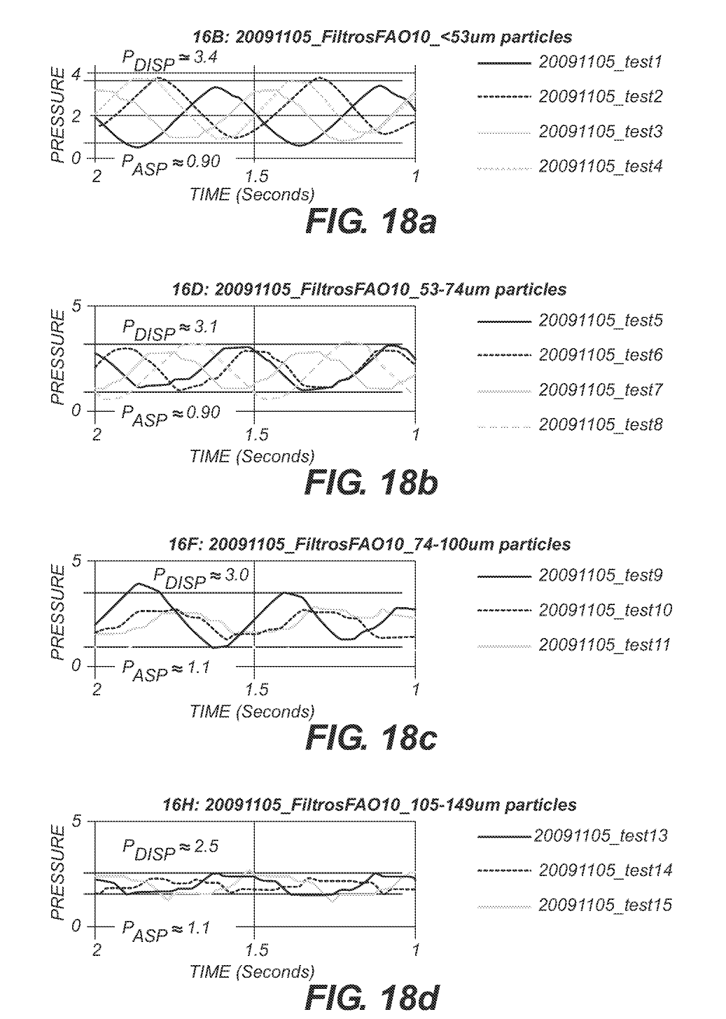

Another aspect of the fluid sample purification system includes the use of particles optimally sized to minimize the effect of accumulation within and against the face of the filter. Particles are forced against the surface of the filter during the sample dispense cycle. In the experiment shown in FIGS. 18a-d, particles sized at less than 53 μm, 53-74 μm, 74-100 μm and 105-149 μm create maximum dispense pressures of 3.4 KPa, 3.1 KPa, 3.0 KPa and 2.5 KPa, respectively. Accordingly, for a filter in the fluid sample purification system in pipette tip format, the particle size is preferably approximately 100-150 μm (or larger), the filter is 1.0 mm diameter and the pore size is 10-40 μm. Particles with such a well controlled size distribution are easily obtained by sieving or other such size segregation methods. It is to be understood that filters with larger or smaller pore sizes could optimally use larger or smaller particles sizes, depending upon the requirements of the specific application.

The fluid sample purification system has a lower and more consistent tip-to-tip resistance to fluid flow than prior art systems. This performance characteristic is particularly important in applications in which multiple pipette tips are attached to a single pipettor with multiple probosci connected to a common aspirate/dispense pressure manifold, wherein the same aspirate/dispense pressure is applied to every pipette tip. Pipette tips that do not contain any internal filters have negligible resistance to fluid flow and are generally known to fill at the same rate and to the same level. However, as resistance to flow increases, any variation in resistance to fluid flow (e.g., a more restrictive distal retainer or filter) will cause the affected pipette tip to receive more or less fluid. In the worst case, one pipette tip could receive little or no fluid while another is tilled to capacity, possibly overflowing and contaminating the pipettor. The fluid sample purification system avoids this problem by better controlling filter flow characteristics (e.g. lower and more consistent filter-to-filter flow resistance), particle induced flow resistance and through the fluid handling protocols described herein, thereby minimizing variation in flow resistance.

The type, size and number of adsorption particles used in the fluid sample purification system help to provide the following benefits:

(a) Reduced purification process time owing to faster fluid aspiration and dispense cycle times.

(b) Reduced purification process time owing to the faster adsorption time as a result of the greater level of agitation caused by the higher fluid flow rates.

(c) More consistent tip-to-tip aspirate and dispense volumes.

### 5.4 Containment of Adsorption Materials or Functionalized Particles within the Housing

In certain embodiments, adsorption materials or particles (e.g., functionalized particles) can be contained (constrained or confined) within the housing (e.g., pipette tip, column) of the fluid sample purification system. Although the description below chiefly discusses the containment of materials within the fluid sample purification system in a pipette tip format, the skilled practitioner will recognize that such means and methods for containment can be applied to other housings, such as column formats, as well.

In one embodiment, particles can be used that are larger in size than the inside diameter of the housing (e.g., pipette tip nozzle, the distal end of the pipette tip) as shown in FIG. 5a.

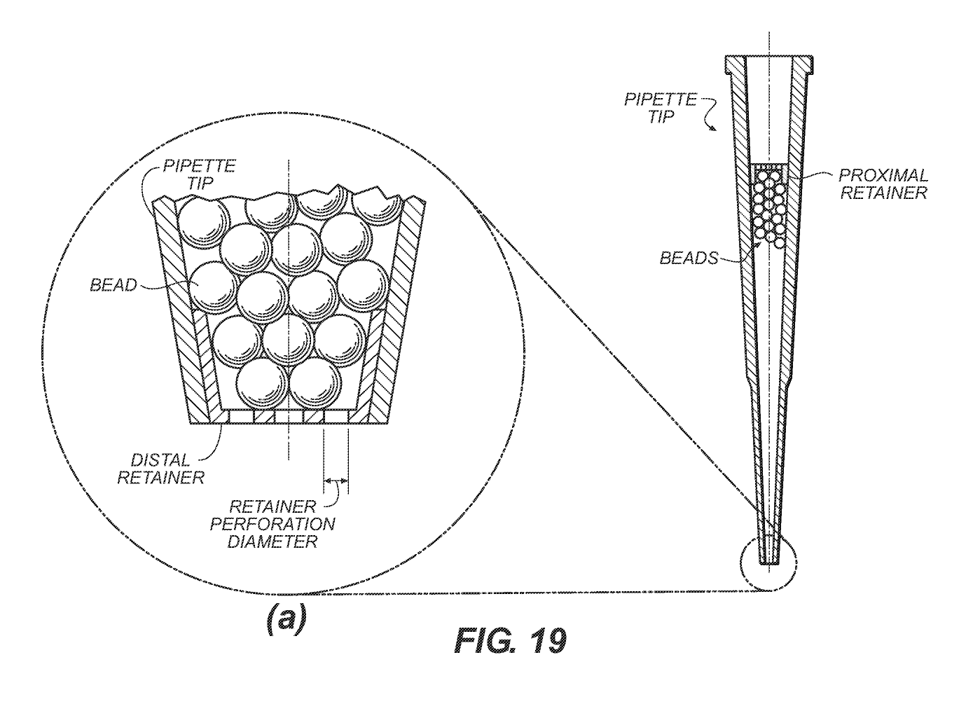

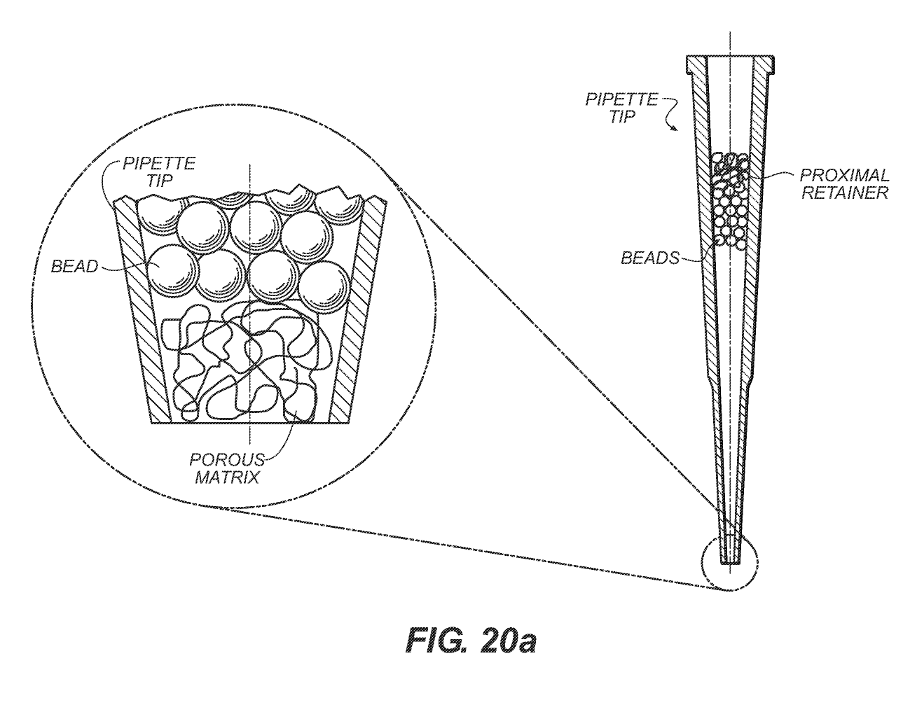

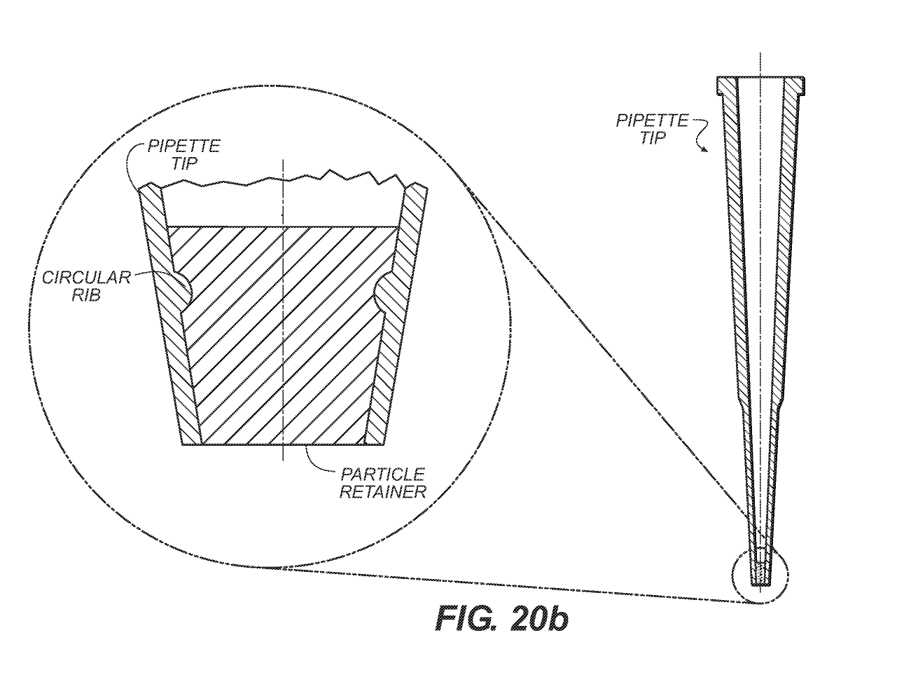

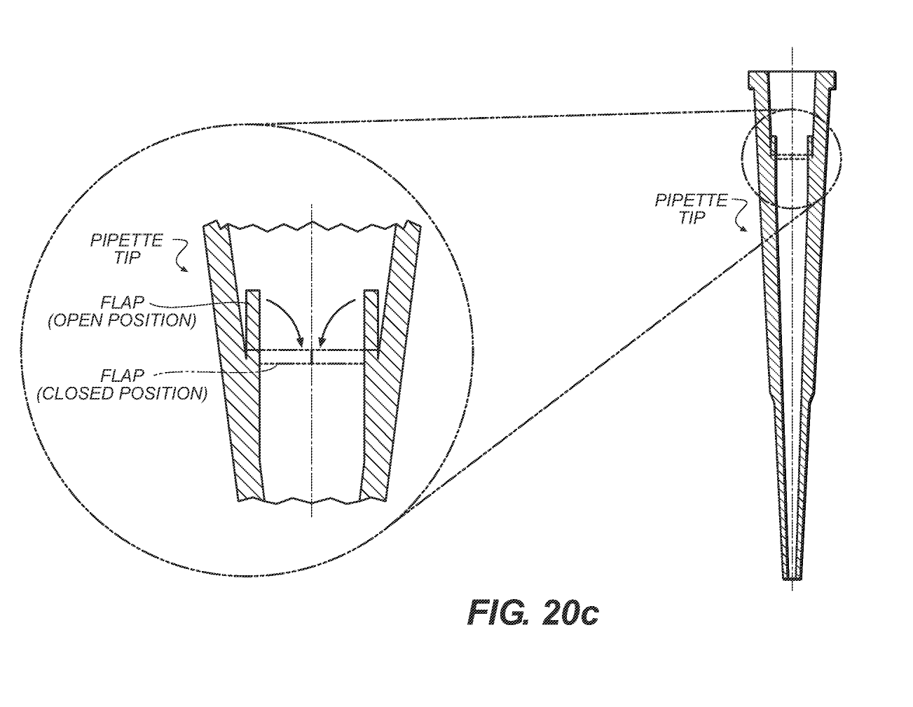

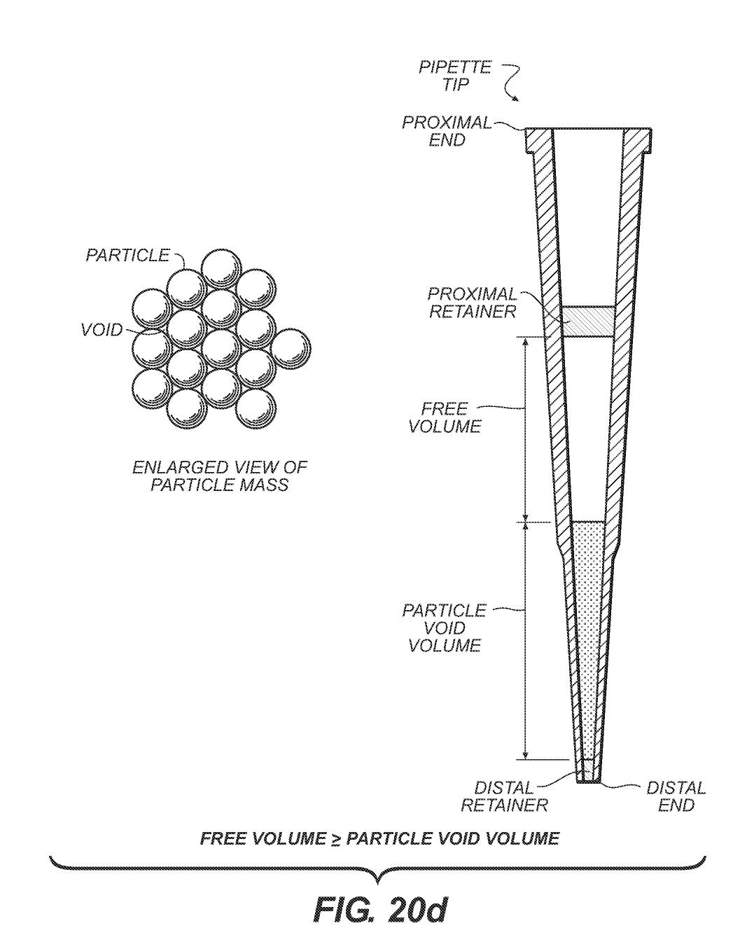

In another embodiment, the distal retainer for the particles or adsorption materials can be porous (e.g., a filter) and can be positioned at the distal (orifice or nozzle) end of the housing, whereby the effective pore size of the particle retainer is smaller than the size of the particles (FIGS. 19 and 20a).

In another embodiment, a thin-film or thick-film particle retainer with perforations smaller than the size of the particles can be used (FIG. 19, see (a) enlarged view). The film particle retainer can be circular or non-circular (e.g., oval or square shaped films).

The use of a porous media, such as a fibrous matrix of biologically inert polypropylene mesh or similar material, wherein the effective pore size of the matrix is smaller than the size of the particles (FIG. 20a). Specific examples of available materials include the hydrophilic filter materials manufactured by Filtrona Fibertec or Ahlstrom. Specific examples of products using these materials include analytical filter papers and membrane backings for liquid chromatography columns and reverse osmosis filters.

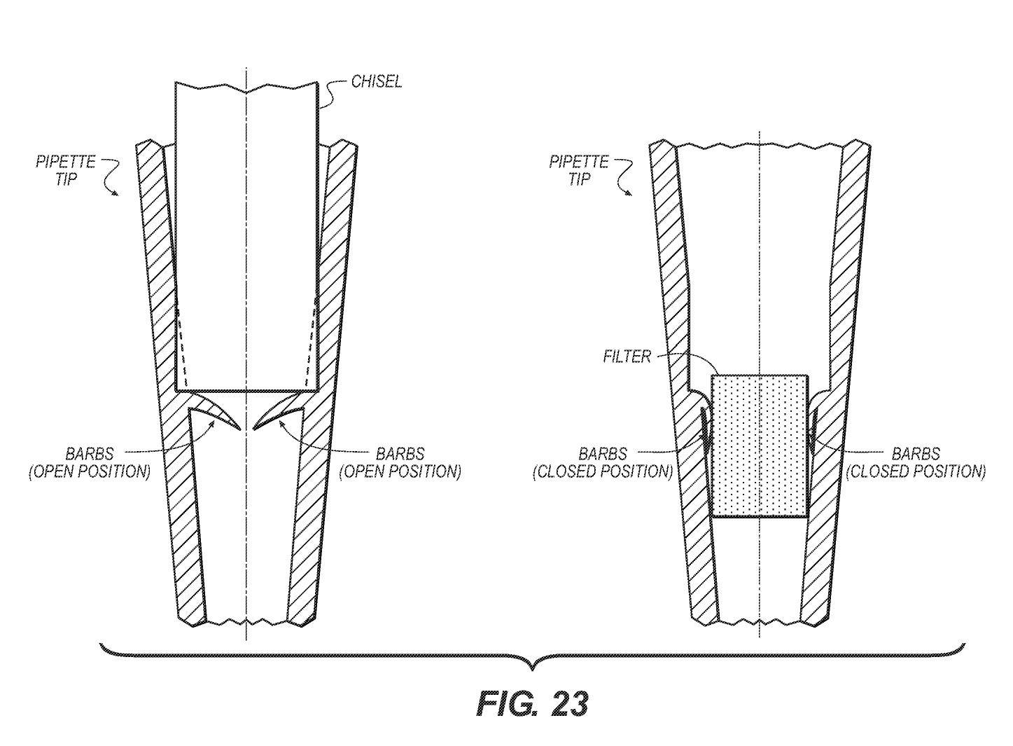

Retainers can be held in place within the housing using internal raised elements, such as circular ribs or barbs, as shown in FIG. 20b, or FIG. 23, which utilize mechanical interference to secure the particle retainer. Preferably, these securing (or retaining) elements (“retention elements”) for the retainer are molded as part of the housing (e.g., pipette tip) but could also include a separate part that is inserted into the housing.

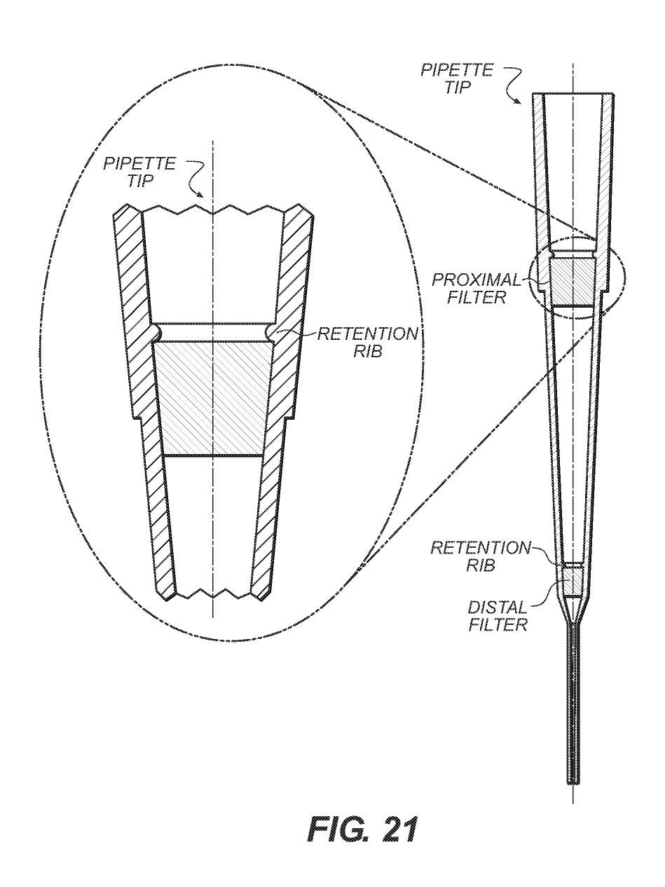

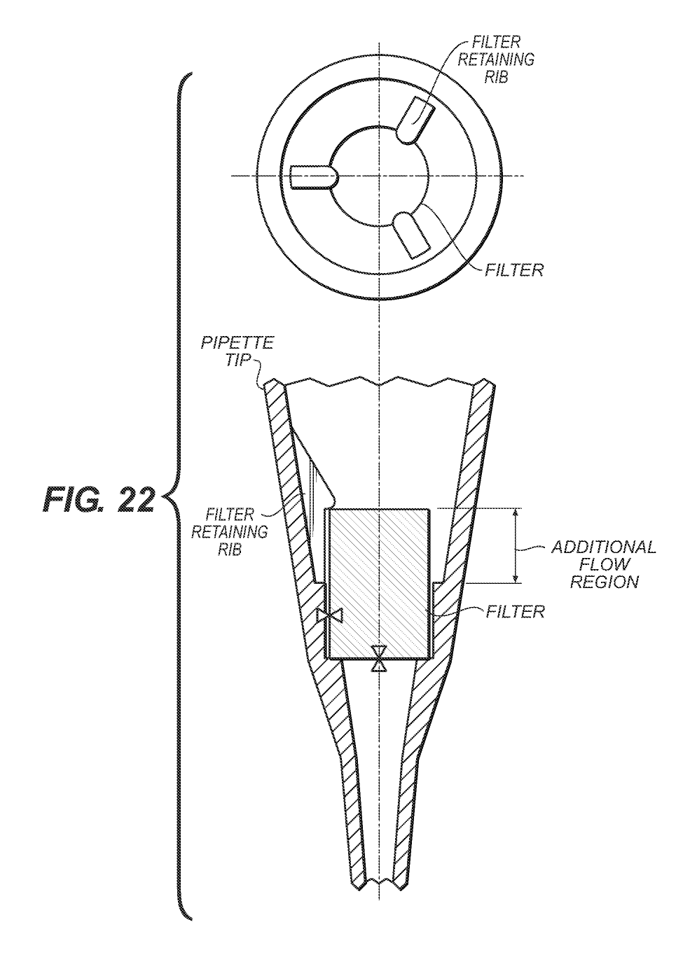

In another embodiment, the fluid sample purification system can comprise a retention element for the internal retainers (e.g., filters) as shown in FIG. 21 or 22. The retention element, such as an internal rib or barb, can be used secure a filter in position as a retainer without the use of high filter insertion forces and the compression of the filter and reduction of filter pore size that can be caused by these forces. The fluid sample purification system can therefore comprise an internal integral design feature to retain the filter(s) within the pipette tip without the need for high filter insertion forces, thereby reducing the likelihood of compressing the filter and increasing fluid flow resistance.

In yet another aspect, the taper of the exterior surface of the particle retainer is similar to the internal taper of the housing so as to, for example, facilitate insertion and retention of the particle retainer as well as to help ensure that particles do not pass between the particle retainer and the internal surface of the housing.

A proximal retainer can be positioned at the rear opening of the housing (e.g., the end of the pipette tip proximal to the pipettor). In one embodiment the proximal retainer can be a porous particle retainer wherein the effective pore size of the particle retainer is smaller than the size of the particles (FIGS. 19 and 20a). As described for the distal particle retainer or filter at the end of the housing, thin-films or thick-films with perforations smaller than the size of the particles can be used (FIG. 19). Porous media can also be used, such as fibrous matrix of biologically inert polypropylene mesh or similar material that acts as a gaseous barrier, wherein the effective pore size of the matrix is smaller than the size of the particles (FIG. 20a). Specific examples include aerosol barrier pipette tips, such as Evergreen Scientific's AeroPure™ pipette tip, which utilizes a hydrophobic filter placed between the sample and pipette shaft to trap and prevent aerosols from reaching the pipette tip holder, while not impeding air flow.

In other embodiments, proximally positioned internal membranes can be used for retention that are collapsed and locked together after particles are placed into the housing (FIG. 20c), wherein the locked membranes provide orifices, such as along interlocking seams, small enough to contain the particles but large enough to allow fluid and/or gas to pass. The membranes are preferably molded as part of the pipette tip but could also include a separate part that is inserted into the pipette tip.

As described for the distal retainer, the proximal retainer can be held in place using internal raised features, such as the circular ribs (FIG. 20b), which utilize mechanical interference to retain the package. Preferably, these features are molded as part of the housing, but could also include a separate part that is inserted into the housing.

In yet another aspect of the proximal retainer, the taper of the proximal retainer is similar to the internal taper of the housing (pipette tip) so as to, for example, facilitate insertion and retention of the particle retainer as well as to help ensure that particles or fluid does not pass between the particle retainer and the internal surface of the pipette tip.

### 5.5 Retainer Structural Characteristics

In certain embodiments, distal and/or proximal retainers can be frits, filters, or comprise filter materials. Retainer materials used in the fluid sample purification system preferably possess certain mechanical strength characteristics to be easily manufactured and to reliably meet their performance requirements while being used. Retainers are preferably capable of being secured into the pipette tip and remaining in place over time. Current product manufacturers typically “press” filters into pipette tips and rely upon the interference between the filter and pipette tip to keep the filter in position. While this is a simple manufacturing process, it does not always work owing to, for example, relaxation of the typically used polymeric filter materials. For example, proximally positioned aerosol filters are known in the art to be prone to falling out of pipette tips.

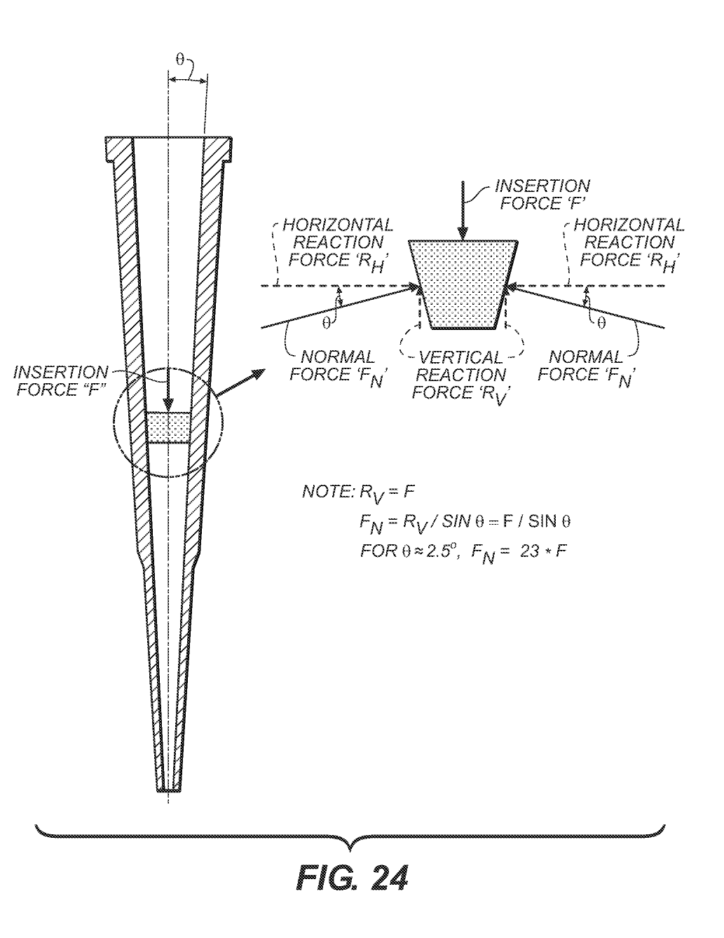

Furthermore, owing to the taper of inner pipette surfaces, the normal forces placed upon the retainer (i.e., forces perpendicular to the exterior surface of the retainer) can be many times higher than the retainer insertion force, as is shown in FIG. 24, and can often compress the retainer, thereby increasing its resistance to flow. Slight variations in retainer insertion forces can therefore produce large variation in normal (compressive) forces on the retainer and therefore the potential for large variation in flow resistance for retainers that cannot resist compression. In many pipette tip applications, such compression may not present a serious problem, particularly when the fluid passing through the retainer is air (e.g., an aerosol filter) which has a very low fluid viscosity. However, when liquids are involved and when consistent tip-to-tip flow rates are required, retainer flow resistance cannot be subject to much variation. For polypropylene filters commonly used in the art, the insertion force required to avoid (indeterminately) deforming the filter is not capable of reliably seating the filter in the pipette tip.

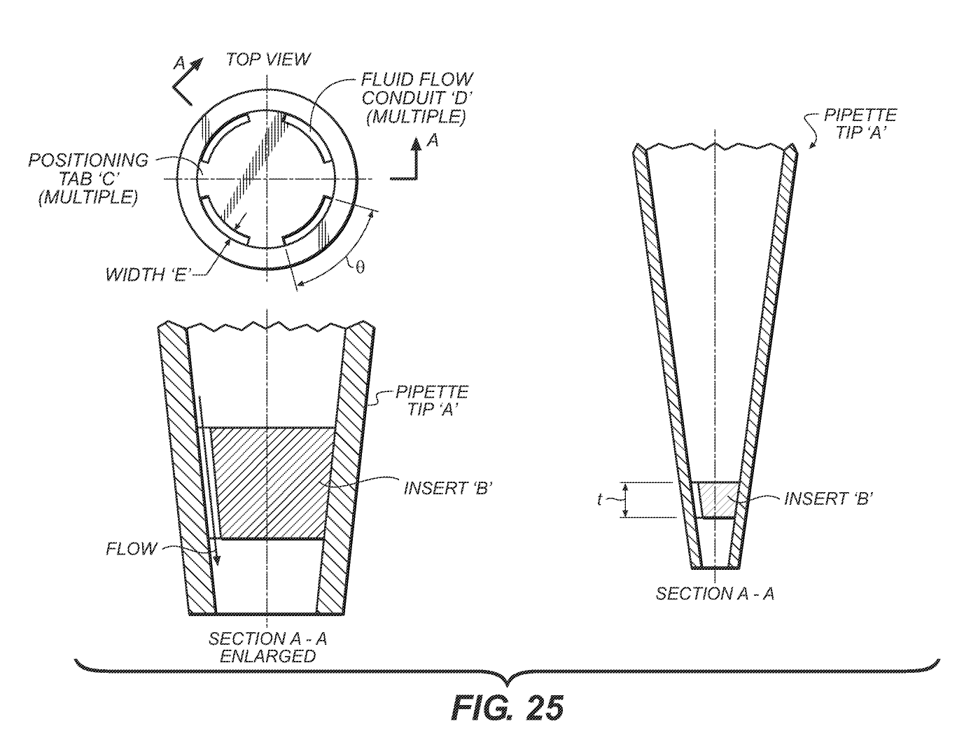

Thus a further aspect of the fluid sample purification system is the use of ceramic filter materials, which provide the mechanical strength (e.g., approximately 7,000-10,000 pounds per square in for ceramic filters and much higher for the retainer shown in FIGS. 25 and 26 to allow high insertion forces and reliable filter positioning (e.g., owing to high mechanical interference between the retainer or filter and the pipette tip), while simultaneously resisting pore compression. In this aspect, the filter or retainer material has the high mechanical strength for reliable positioning while simultaneously satisfying system performance needs. Such a filter or retainer can form an incompressible (or almost incompressible) “plug” in the distal opening (e.g., the pipette tip).

In certain embodiments, particle retention or confinement within the fluid sample purification system is accomplished by retainers that are filters. Filter characteristics of the fluid sample purification system include pore or flow channel characteristics (e.g., dimensions), the physical size of the filter, low intrusion pressure so as to easily initiate flow, low extrusion pressure so as to help simplify removal of fluid sample, propensity to adsorb DNA, and structural characteristics.

The pores in the distal particle retainer or filter must be large enough to enable high fluid flow rate (e.g., help to minimize purification time at the relatively low pressures provided by laboratory pipettors), yet small enough to retain the functionalized particles.

In one preferred embodiment, the particles size is 50-100 μm (in diameter, if roughly spherical, or in maximum width or length, if not spherical), which is based upon the system requirements for rapid purification time, minimization of retained fluid sample volume and ease of product manufacturing. The fluid sample purification system can therefore comprise a filter that retains particles of this size, specifically a filter with an effective pore size smaller than 50 μm in diameter. However, it is well known in the art that steady-state flow rate decreases with decreasing pore size for a given set of fluid properties and applied pressure. Therefore there exists an optimal pore size that meets particle retention requirements while maximizing fluid sample flow rate at the applied pressures provided by typically used pipettors.

It is well known in the art that increasing pore size in 1.0 mm diameter distal filters increases the rate of flow through the filter. Likewise, Table 2 shows that the 1.0 mm diameter filters with pore sizes of 10 μm are able to effectively retain optimally sized particles (i.e., particles between 50 and 100 μm in diameter). However, the rate of fluid flow through the filter is higher than for existing art-known filters, owing in part to the filter having an unusually large pore size for a filter of this size. While other pipette tip manufacturers use similarly sized or even larger diameter filters, they are unable to achieve high flow rates comparable to the fluid sample purification system provided herein. FIGS. 12a-c show fluid aspirate and dispense pressure-versus-time curves for a representative commercially available filtered pipette tip (Thermo Fisher Scientific's Aspire™ product).

TABLE 2
Particle Retention by Filter

| | | Tube Weight (g) | | |
|---|---|---|---|---|
| | | before | after | difference |
| | Sample | treatment | treatment | (mg) |
| 1 | empty control tube | 0.4682 | 0.4681 | −0.1 |
| 2 | empty tube + 50 μl | 0.4756 | 0.4758 | 0.2 |
| 3 | water | 0.4711 | 0.4713 | 0.2 |
| 4 | | 0.4727 | 0.4730 | 0.3 |
| 5 | tube + 50 μl water | 0.4761 | 0.4762 | 0.1 |
| 6 | treated with DG | 0.4693 | 0.4693 | 0 |
| 7 | prototype | 0.4734 | 0.4737 | 0.3 |

As shown in Table 2, the embodiment of the fluid sample purification system (comprising Axygen tip and FAO10 filter) retained silica particles (53-74 μm average size). Tubes were pre-weighed and filled with 0 or 50 μl water. Fluid sample purification systems (“prototypes”) were filled with 5 mg unfunctionalized particles (53-74 μm) and 50 μl water was repeatedly aspirated and dispensed for 15 cycles in the original tubes. Tubes were then covered with a laboratory wipe and allowed to air dry (˜96 hours) before being weighed again. Alter treatment, on average control samples 2, 3, and 4 were 0.231 mg heavier and experimental samples 5, 6, and 7 were 0.2 mg heavier suggesting that no particles leaked through the filter during treatment.

Similar data for one embodiment of the fluid sample purification system are shown in FIGS. 13a-c. These curves indicate a higher flow rate through the distal filters of the fluid sample purification system, relative to that of the prior art (FIGS. 12a-12c). Features of this embodiment of the fluid sample purification system that contribute to enhanced performance include the use of 20 μm and 40 μm pore size in a very small diameter filters (e.g. 1.0 mm in diameter) and shorter length filter (e.g. 1.0 mm to 1.5 mm in length), as well as fewer functional particles and fewer filters. Filter physical size parameters are disclosed hereinbelow. The pore size and filter size used in this embodiment of the fluid sample purification system create a rate of fluid sample flow that enables short fluid aspirate and dispense cycles and a high level of fluid agitation, which alone or together help to significantly reduce sample purification time, while simultaneously meeting previous system design requirements (e.g., effectively retain 50-100 μm sized particles).

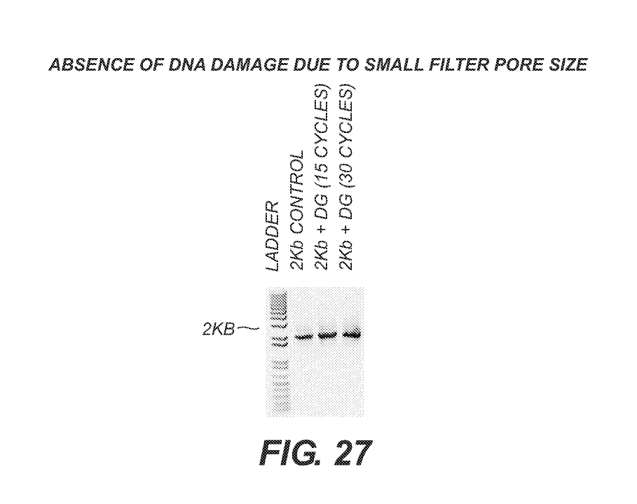

The choice of filter pore size is further constrained by the need to avoid damaging the target or filter material. (e.g., ds-DNA). FIG. 27 shows that the filter pore size and fluid sample flow rate achieved by the described conditions do not damage (i.e., shear) ds-DNA. By contrast, passing ds-DNA through similar sized orifices under less optimal (i.e., higher) flow rates is known in the art to effectively shear ds-DNA.

In FIG. 27, DNA is treated with an embodiment of the fluid sample purification system in pipette tip format (“Diffinity RapidTip” embodiment). FIG. 27 shows that DNA showed no evidence of shear damage after either 15 or 30 aspiration-dispense cycles. An approximately 2 Kb (1956 bp) PCR product was generated from lambda DNA template and custom primers (forward: TGA AAC GCT TGC TGC AAC GCC AAA [SEQ ID NO: 1] and reverse: AAA GCA ATT GGC GGT GAT GTA AAC ACT ATG [SEQ ID NO: 2]). The stock sample was split for control and treatment samples. DG treatment was performed with 25 μl of DNA sample in a fluid sample purification system in pipette tip format, which contained 2.5 mg unfunctionalized material (Axygen tip, FAO10 filter-600 g insertion). The sample was alternately aspirated and dispensed either 15 or 30 times. Equivalent volumes (5 μl) of control and treated samples were run in adjacent lanes on a 1% agarose gel that also contained a standard DNA ladder (Invitrogen 1 Kb Plus). Typical treatment requires about 15 aspiration-dispense cycles resulting in a 2-fold safety margin.

The fluid sample purification system therefore provides optimum filter pore size for retaining optimally sized particles, while simultaneously enabling a high fluid flow rate through the filter at low applied pressures (i.e., those available from typical pipettors) and not damaging the filtered material (e.g., ds-DNA).

Like other sub-system components in the fluid sample purification system, the physical size of the distal filter is subject to competing design requirements. The filter is preferably small enough to minimize sample dead volume (i.e., enable placement of the filter nearer the distal end of the pipette tip) and to minimize retained sample volume (i.e., possess a minimal void volume and low extrusion pressure). At the same time, the filter must be large enough in cross-sectional area to provide low resistance to fluid flow (e.g., a larger number of pores for fluid to pass through) and ease of manufacturability. Lower fluid dispense rates serve to minimize agitation within the pipette tip, which in turn could decrease the level of purification achieved and therefore the level of purified sample quality or alternatively, the time (i.e., number of sample aspiration/dispense cycles) required to achieve a specific level of sample quality.

The filter preferably maintains a consistent pressure drop across multiple filters, which is desired when using any pipette tip (functional or otherwise) in a fluid handling system that uses multiple pipette tips at one time, such as a multi-channel pipettor. Each pipette tip in such a system is typically linked to a common vacuum/pressure manifold that applies the same vacuum/pressure to each pipette tip for the same period of time during fluid aspirate/dispense cycles. Differences in vacuum/pressure drop across the filters used in one or more of the pipette tips can result in a pipette tip aspirating and dispensing more or less fluid during the aspirate/dispense cycle. This can result in either insufficient purification in the low-flow pipette tip, over flowing the high-flow pipette tip, or both.

The fluid sample purification system overcomes these conflicting requirements in several ways. A filter pore size can be used that is large (e.g. 20 μm and 40 μm) for the maximum filter diameter requirements (i.e., on the order of 1-2 mm) associated with functional pipette tips and the very small sample volumes (e.g., 5-50 μl) they are often required to handle. In one embodiment, a filter material (e.g., ceramic) can be used that provides the structural strength required to reliably maintain pore size, including when subjected to high filter insertion forces, particularly the forces normal to the surface of the filter caused by internal pipette tip taper as shown in FIG. 24, as well as during filter manufacturing and assembly. In another embodiment (FIG. 25 and FIG. 26) the filter is replaced by an engineered flow duct that does not require flow through a filter. In this embodiment, there is no porous member that is subject to compression by insertion forces.

A filter can be used with shorter overall length and hence lower resistance to fluid flow. Resistance to flow through a porous filter increases with the length of the filter, owing to the longer path through the filter. At the same time, it is desirable for manufacturing purposes to design filters that have different diameter and length dimensions (e.g., a length that is 50% longer or shorter than the filter diameter) which can be used to help orient the filter during assembly. Therefore, yet another element of the fluid sample purification system and methods is the ability to create and use a filter that is, for example, only 1.0 mm in length, that enables for example, a 0, 50 or 1.5 mm length and therefore low resistance to fluid flow than a filter with greater aspect ratio. In one embodiment, a single filter can be used.

In another embodiment (FIG. 26) the filter is replaced by an engineered flow duct that utilizes a solid retainer, whereby the high structural strength of the solid (i.e. non-porous) retainer enables it to be very thin, e.g. 0.10 to 1.0 mm thick for an inner pipette diameter of 2-6 mm, thereby providing further reduction in fluid flow resistance.

FIGS. 5a and 5b describe the use of particles that are larger in size than the inside diameter of the pipette tip nozzle (i.e., the distal end of the pipette tip), wherein internal, axially running ribs contain the particles while simultaneously allowing fluid to pass around the exterior of the particle, into and out of the pipette tip. The ribs may consist of a feature molded into the pipette tip or a separate part that is inserted into the pipette tip to provide the same particle retention function. This latter feature provides the additional advantage of preventing spherically or near-spherically shaped particles from plugging the nozzle of the pipette tip.

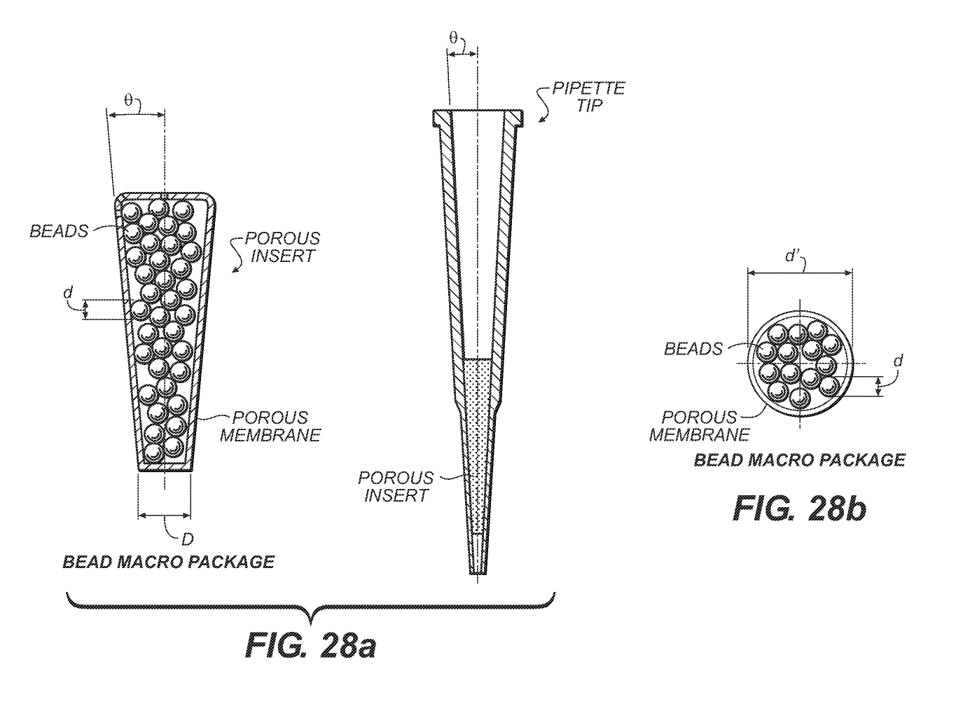

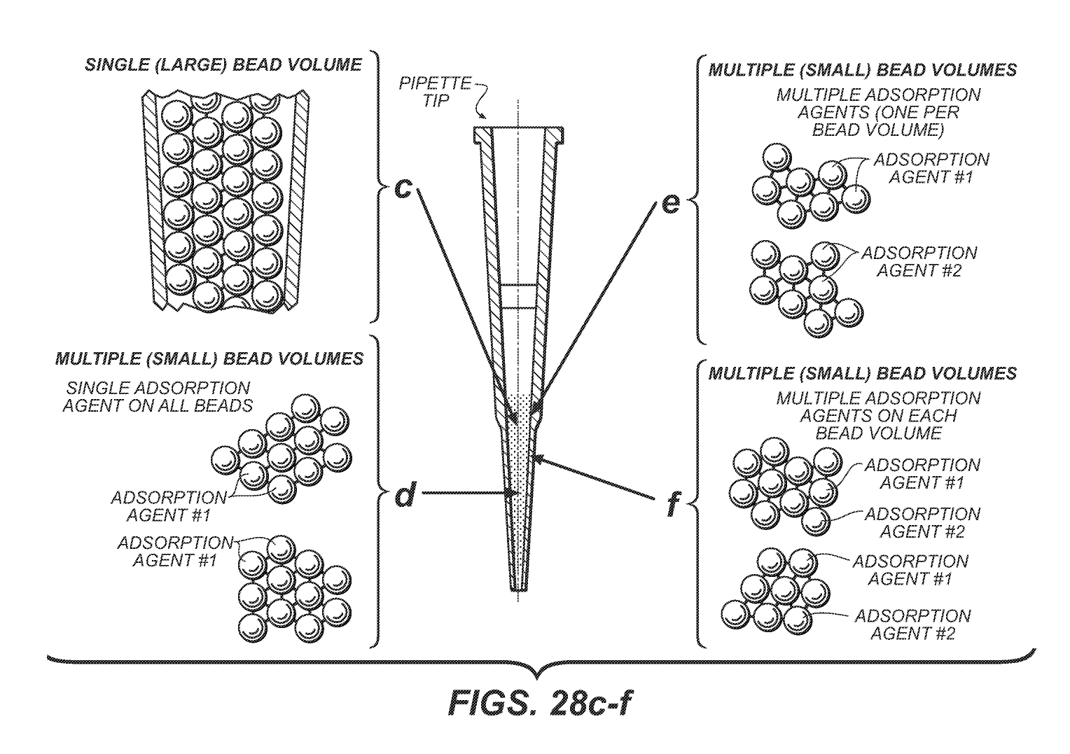

FIGS. 28a-h show the use of a porous package, analogous to a “tea bag,” which can be placed into a pipette tip. In this embodiment, the package consists of a porous film, such as a biologically inert polypropylene mesh or similar material, wherein the effective pore size of the mesh is smaller than the size of the particles retained within the package.

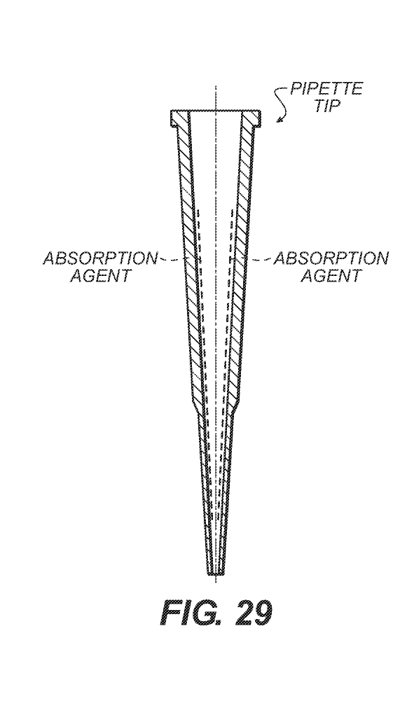

FIG. 29 shows that an adsorption material or agents can be attached to the inside surface of the pipette tip, thereby eliminating the need for any filter.

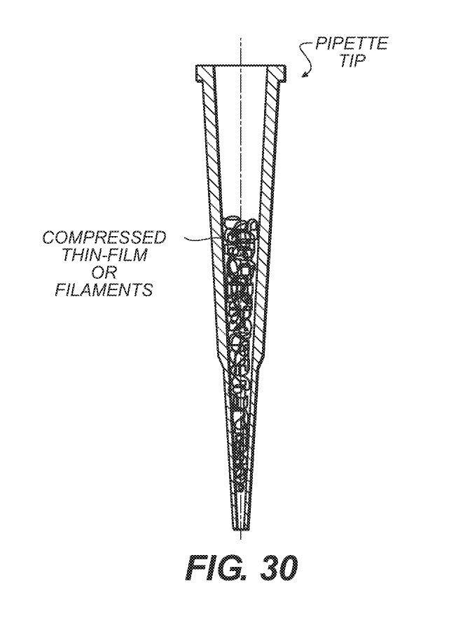

FIG. 30 shows a further improvement wherein the adsorption material or agents are attached to the surface of a film which is compressed into a small volume or multiple volumes and placed inside the pipette tip. The compressed film is prevented from passing through the distal end of the pipette tip by using a compressed film volume that is larger than the maximum diameter of the pipette nozzle, thereby eliminating the need for any filter.

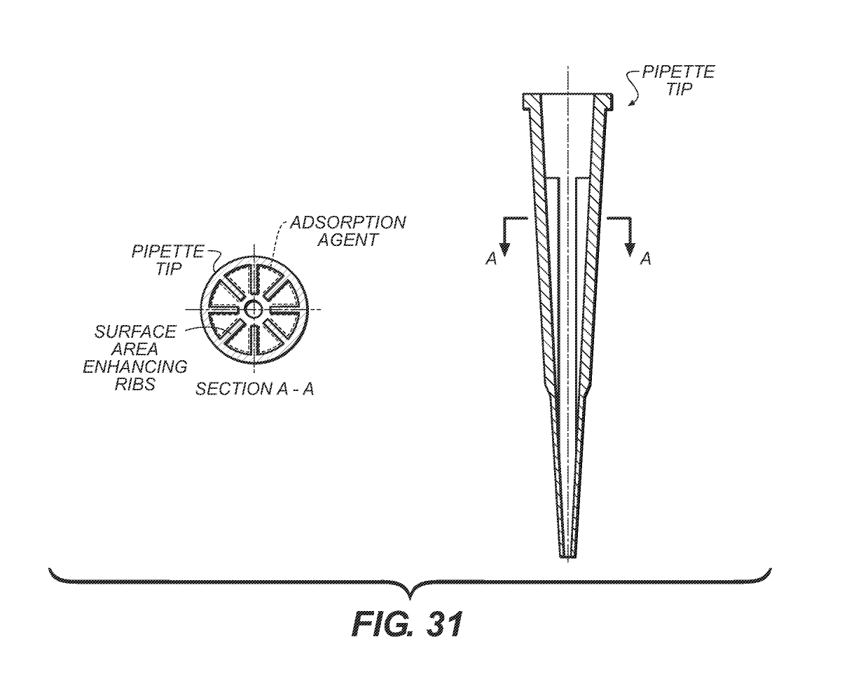

FIG. 31 shows a further improvement wherein the adsorption material or agents are attached to multiple features within the pipette tip that serve to increase wettable surface area, such as multiple, internal, axial running ribs. The ribs may be an integral part of the pipette tip (i.e., a molded feature of the pipette tip) or a pre-formed member that is placed inside the pipette tip before molding (i.e., insert molding), or after molding, thereby eliminating the need for any filter.

The absence of a second filter in certain embodiments can thereby reduce the resistance to fluid flow (either sample liquid or pipettor air) caused by the second filter. Likewise, the fluid sample purification system's use of freely suspended particles eliminates the need for the longer column (i.e., chromatography resin) used by the aforementioned Zip-Tip, owing to the limited number of particles the Zip-Tip product embodiment can place in a given length of filter.

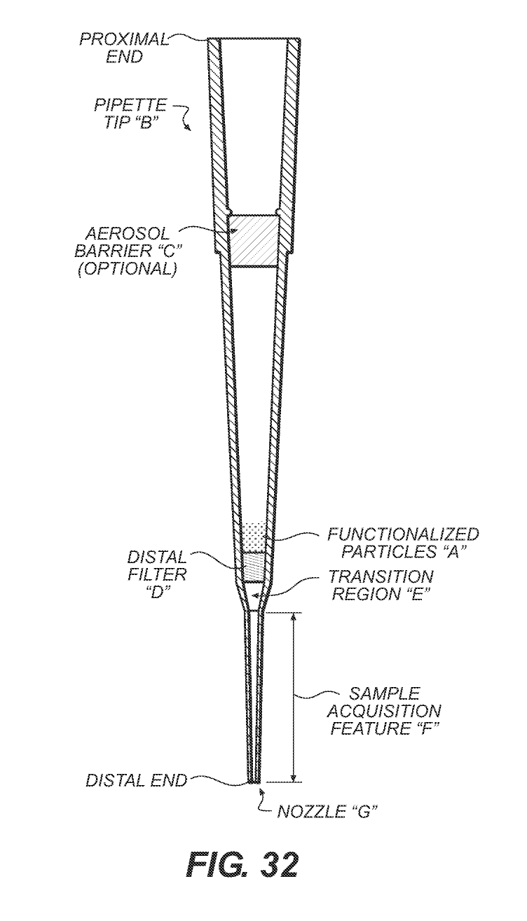

In another embodiment of the fluid sample purification system, the pipette tip format shown in FIG. 32 can also be used. This design accommodates filters with larger cross-sectional area and hence lower resistance to fluid flow, while simultaneously minimizing sample dead volume, by using a small bore at the pipette nozzle and rapidly increasing the internal diameter of the pipette tip to accommodate the larger filter.

Since many sample processing procedures typically use fluid sample volumes of the order of 5-50 μl, filter cross-sectional area cannot be made arbitrarily large, owing in part to the competing need to minimize internal void volume and the potential to retain sample volume. Subject to the pore size limitations imposed by the need to retain optimally sized particles, filters smaller than 1.0 mm in diameter are difficult to manufacture and do not possess a sufficient number of pores to create a sufficiently high fluid sample flow rate. Also, increasing filter size can trap excessively large volumes of fluid sample. A filter that is 1.0 mm in diameter and 1.5 mm long (i.e., a filter suitable for processing sample volumes of the order of 5-10 μl) with a void fraction of 50% will have a void volume of 0.6 mm³ or 0.6 μl, or approximately 12% of a 5.0 μl sample size. Likewise, a filter that is 2.0 mm in diameter and 3.0 mm long (i.e., a filter suitable for processing sample volumes of the order of 10-25 μl) with a void fraction of 50% will have a void volume of 4.7 mm³ or 4.7 μl, or approximately 18% of the total sample for a 25 μl sample size. By comparison, a filter that is 3.0 mm in diameter and 4.50 mm long i.e., a filter suitable for processing sample volumes of the order of 25-50 μl) with a void fraction of 50% will have a void volume of 15.9 μl, which would be approximately 32% of a 50 μl sample volume, an unacceptably high loss even for a sample volume of this size.

While the fluid sample purification system can more effectively recovering sample volume for the reasons previously indicated, there may exist applications (e.g., the need for filters with hydrophobic surfaces and as a result, higher fluid extrusion pressures) that cannot use filters with larger pore size or with hydrophilic surfaces. For example, large filter pore size and filters with more hydrophilic surfaces may not always be desired for a specific end-application, and therefore filter size must still be considered. Since many sample processing procedures typically use fluid volumes of 5-50 μl, a distal filter diameter of 1.0-3.0 mm can be used for processing sample volumes of the order of 5-50 μl, which represents the maximum loss of sample volume that could be tolerated for most applications.

This range of filter sizes also provides the ability to achieve high sample flow rates at typical pipettor pressures, while simultaneously retaining optimally sized particles. Furthermore, since the low pressure drop created by this size of filter is small relative to the pressures produced by typically used pipettors, filter-to-filter variation in pressure drop, owing to for example, manufacturing process variability, will also be small, thereby helping to ensure an acceptable level of filter-to-filter variation in pressure drop, and as a result, more consistent flow performance across different pipette tips.

Thus, the pipette tip and distal filter design of the fluid sample purification system enable it to achieve high sample recovery, high flow rate, more uniform tip-to-tip flow rates (e.g., aspirate and dispense rates), and ease of manufacture, while simultaneously satisfying previous system performance requirements.

In one embodiment, the fluid sample purification system uses a distal retainer design that provides low resistance to fluid flow and low target (e.g., DNA) adsorption characteristics. Also, by not containing its adsorption materials within a porous matrix (e.g., Millipore's Zip-Tip), the fluid sample purification system also avoids the high flow resistance associated with the relatively large porous matrices required to contain a sufficient volume of adsorption material. The increased rate of fluid flow associated with this aspect of the fluid sample purification system provides benefits including:

(1) Reduced purification process time owing to faster fluid aspiration and dispense cycle times.

(2) Reduced purification process time owing to the faster adsorption time as a result of the greater level of agitation caused by the higher fluid flow rates during sample aspiration and dispensing.

(3) Reduced product manufacturing cost by eliminating a potentially expensive component (e.g. Millipore's porous filter with integrated adsorption chemistry).

Although not directly related to fluid flow rate, the fluid sample purification system uses a distal filter that avoids reduced target yield owing to the adsorption of target materials by the large surface area associated with purification systems that attach adsorption materials to the surfaces of a porous matrix, owing to the large surface area of the porous matrix relative to the surface area of the adsorption materials contained therein. Also avoided is damage to target materials (e.g., shearing target DNA) owing to the passage of sample through the relatively large porous matrices used to contain adsorption materials in this manner.

In a preferred embodiment, the fluid sample purification system comprises a distal retainer that has very low fluid flow resistance, consistent fluid flow resistance, uniform fluid flow duct dimensions, low sample retention and low cost. Unlike prior art fluid sample purification systems, the embodiment of the fluid sample purification system with this distal particle retainer design has features within the pipette tip, rather than a separate filter, to provide the particle retention function.

In FIG. 25, pipette tip ‘A’ includes insert ‘B of thickness 1’ positioned within pipette tip ‘A’ by insert positioning tabs ‘C’ creating fluid flow conduits ‘D’ of width ‘E’. Cross-section A-A through pipette tip ‘A’ illustrates fluid flow through the axially-running fluid flow conduits ‘D’ formed by the space between pipette tip ‘A’ and insert ‘B’. Conduit width ‘E’ is precisely determined by the recess in insert ‘B’, such as for example, by the injection mold that precisely forms this feature of the part during the pipette tip injection molding process. The width ‘E’ of flow conduits ‘D’ are chosen to minimize fluid flow resistance, while in one application preventing larger objects (e.g. particles) from passing into or out of pipette tip ‘A’.

In FIG. 26, pipette tip ‘A’ includes insert ‘B of thickness ‘t’ positioned within the pipette tip by insert positioning tabs ‘C’ creating fluid flow conduits ‘D’ of width ‘E’. Unlike the embodiment of the fluid sample purification system shown in FIG. 2, the flow conduits are created in the inner wall of pipette tip ‘A’. Cross-section A-A through pipette tip ‘A’ illustrates fluid flow through the axially-running fluid flow conduits ‘D’ formed into the wall of pipette tip ‘A’. Conduit width ‘E’ is precisely determined by the “core” of the pipette tip injection molds that precisely forms the internal surfaces of the pipette tip ‘A’ during the pipette tip injection molding process. As before, the width ‘E’ of flow conduits ‘D’ are chosen to minimize fluid flow resistance, while in one application preventing larger objects (e.g. particles) from passing into or out of pipette tip ‘A’.

This filter design has the ability to effectively retain particles within the pipette tip. A conduit dimension of, for example, 0.002 inches corresponds to 50 micro-meters, which would effectively prevent the passage of particles 50 micro-meters or larger. Particles are frequently in various ways when processing fluids. Depending upon the size, shape, specific gravity and level of agitation, particles from 50 to 500 micro-meters or larger are often used to process fluids. Accordingly, a preferred conduit width ‘E’ is 0.002 inches to 0.020 inches (i.e. 50 to 500 microns) with 0.002 to 0.006 (50 to 150 microns being most optimal.

This particle retainer design has the ability to minimize flow resistance. It is well known that larger flow cross-sections provide less resistance to fluid flow. The hemispherical shaped conduits created between pipette tip ‘A’ and insert ‘B’ in this embodiment of the fluid sample purification system provide a large flow-through area and therefore low resistance to fluid flow, relative to, for example, a porous filter such as a porous frit. In FIG. 25 and FIG. 26, a pipette tip inside diameter of 0.060 inches, an insert with four 0.010 inch wide positioning tabs that create a conduit width of 0.004 inches provides a total flow cross-sectional area that is 0.000554 square inches. By comparison, the flow cross-section of a 0.020 inch diameter pipette tip nozzle is 0.000314 square inches. In this specific example, the cross-sectional area of the flow conduit is 1.76 times larger than that of the unrestricted pipette nozzle. Likewise, the flow conduit of this embodiment is also much larger than the effective flow cross-sectional area of any porous frit or membrane of the same size. Furthermore, the straight-through flow path design of this embodiment creates less resistance to fluid flow than an equivalent thickness of porous material, such as a porous frit. The design minimizes the thickness ‘t’ of flow restrictor ‘B’ and therefore minimizes flow resistance.

It is well known that resistance to flow through a conduit is proportional to the length of the conduit, with shorter lengths producing less resistance to flow. Accordingly in the present invention, the length of conduit, ‘t’, is preferably less than the diameter of the conduit (e.g. internal diameter of a pipette where the insert is placed), with a length between 10% and 50% of the size of the insert being most optimal.

Further characteristics of this preferred embodiment are discussed in Section 6.1 (Example 1).

### 5.6 Loosely Packed Particles for Enhanced Adsorption Efficiency

In another embodiment, loosely packed particles or adsorption materials can be used within the housing. Unlike existing flow-through purification devices, such as liquid chromatography columns, the adsorption materials of the fluid sample purification system are free to move within the solution aspirated into the pipette tip to increase their likelihood of encountering a greater quantity of the desired material to be adsorbed. The adsorption capacity of the particles is thereby increased, their ability to more completely remove materials from solution (i.e., adsorption effectiveness) is increased while reducing the time required to purify the sample (i.e., process time). Preferably, the particle volume within the housing (pipette tip) (FIG. 20d) is such that free volume within the pipette tip (i.e., the internal volume not containing particles identified in FIG. 20d as the “Free Volume”) is greater than or equal to the void volume around the particles (identified in FIG. 20d as the “Particle Void Volume”). This ensures that the sample fluid that enters the void volume around the particles when fluid is aspirated into the pipette tip can be completely replaced at least once by fluid not initially exposed to the particles. This helps to ensure that sample fluid does not stagnate around the particles within the pipette tip thereby increasing the likelihood that the adsorption materials immobilized on the particles will encounter and bind with the material to be removed from the sample solution.

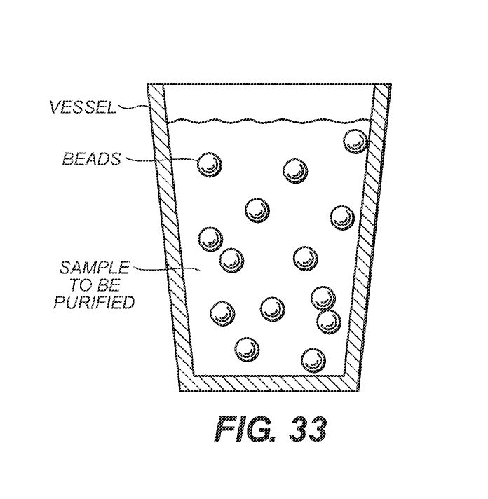

In another embodiment, the adsorption material or agents are placed upon particles (as described herein) which are either added to a vessel containing the sample to be purified, or contained in the vessel prior to adding the sample fluid, whereby they are allowed to attach to materials to be removed from the solution (e.g., single-stranded DNA), such as shown in FIG. 33. The vessel can be any suitable vessel (or plurality of vessels) known in the art for containment of a fluid sample, e.g., test tube, microwell plate, microfuge tube, PCR reaction vessel, etc. The particles containing the materials separated from the sample are then separated from the sample (e.g., impurities) by any one or more of several particle separation methods, such as filtration, sedimentation, centrifugation or magnetic separation. In the latter case, the particles would have magnetic properties.

### 5.7 Pipette Nozzle and Particle Retainer Orifice Sizes and Fluid Dispense Velocities

The need to retain a specific particle size in the pipette tip dictates a maximum pipette tip nozzle size and/or maximum pore size of the distally positioned particle retainer. High fluid shear rates are known to damage analytes in the fluid sample such as nucleic acids (see Bowman and Davidson, Biopolymers 11, 2601 (1972), Hydrodynamic shear breakage of DNA; Yew and Davidson, Biopolymers 6, 659 (1968); Balbi et al., A simple model for DNA elution from filters, Evensen, Meldrum and Cunningham, Review of Scientific Instruments, Volume 69, Number 2, 1968, Automated fluid mixing in glass capillaries). Therefore, optimum or suitable combinations of pipette nozzle and/particle retainer orifice sizes and fluid velocities exists that enable the pipette tip containing the particles to efficiently transfer fluid without damaging the analyte.

Accordingly, in another embodiment, the combination of the size of the orifice in the pipette tip (i.e., nozzle diameter) or particle retainer (e.g., thin-film perforation diameter), and fluid dispense rate are chosen to simultaneously contain the particles while maintaining fluid shear forces that are low enough to avoid damaging DNA. Analyses used to determine fluid shear stress for a combination of pipette tip nozzle or particle retainer perforation diameters and fluid flow rates are known in the art (see Bowman and Davidson, Biopolymers 11, 2601 (1972), Hydrodynamic shear breakage of DNA; Yew and Davidson, Biopolymers 6, 659 (1968); Balbi et al., A simple model for DNA elution from filters, Evensen, Meldrum and Cunningham, Review of Scientific Instruments, Volume 69, Number 2, 1968, Automated fluid mixing in glass capillaries).

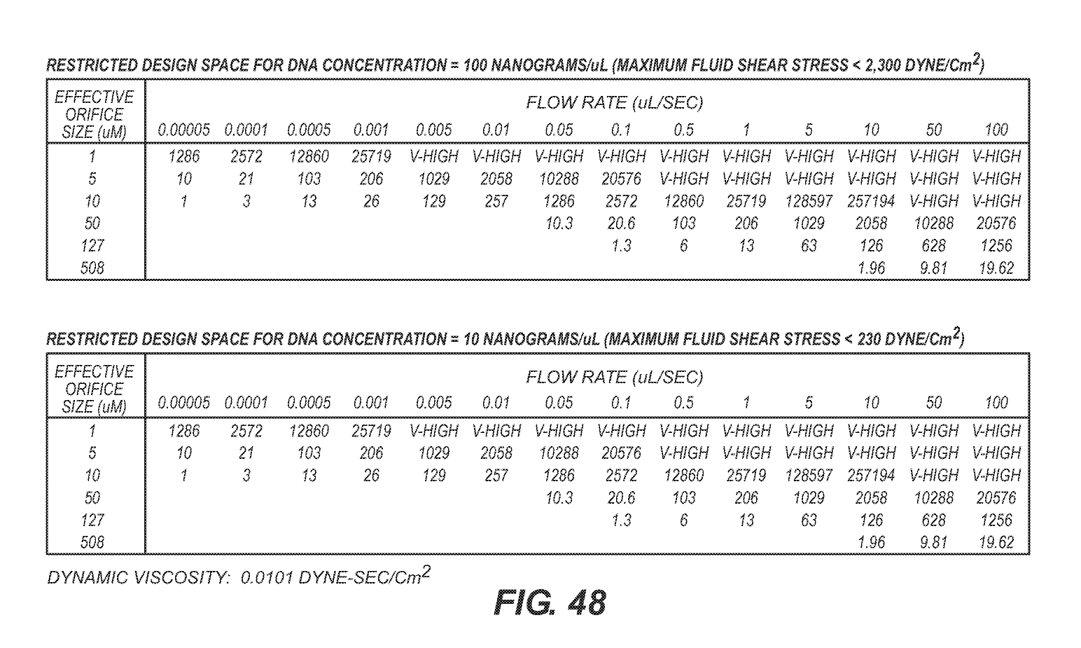

FIG. 48 shows a summary table of fluid shear rates for a wide range of pipette tip nozzle or particle retainer perforation diameters (columns) and fluid flow rates (rows). Based upon Bowman's report of the maximum allowable shear stress a fluid can experience without damaging DNA, a range of allowable combinations of pipette tip nozzle or particle retainer perforation diameters and fluid flow rates is presented in FIG. 48 for two different DNA concentrations; typical post-PCR DNA amplification concentration (typically 100 ng/nμl) and typical DNA sequencing sample concentrations (typically 10 ng/μl). According to Bowman, the latter sample concentrations can tolerate shear stresses of no more than approximately 2,300 and 230 Dynes/Cm², respectively. The table in FIG. 48 specifies the allowable combinations of pipette tip nozzle or particle retainer perforation diameters and fluid flow rates that can be used without damaging DNA in the fluid sample.

For example, referring to the table in FIG. 48, for a sample DNA concentration of 100 ng/μl: A distal particle retainer consisting of a thin-film with 10 μm pore sizes could tolerate a flow rate of no more than 0.05 μl/sec across through each pore in the retainer, which would produce the indicated fluid shear stress of 1286 Dynes/Cm². Assuming that the thin-film retainer contained approximately 2.5*10⁹ pores per square meter, a pipette tip with a 0.50 millimeter orifice would contain 508 such pores. The analysis set forth in “Pipette Tip Nozzle Flow Considerations” (below) can be used for this calculation.

Therefore, the maximum fluid flow rate through such a retainer would be (508 pores)*(0.05 μl/sec) or 25.4 μl/sec. For a typical 50 μl sample volume, this particular pipette tip and particle retainer design would necessitate taking approximately two seconds to aspirate the sample fluid into the pipette tip (i.e., 50/25.4).

Pipette Tip Nozzle Flow Considerations

It is well known in the art that nucleic acids, particularly DNA, can be damaged (i.e. “sheared” into shorter lengths) by exposing the DNA-bearing solution to high fluid shear forces. The following analysis examines fluid flow through a pipette tip nozzle and permeable fabric to establish flow conditions that help to minimize or eliminate such damage.

Case I: Shear stress for flow through a pipette nozzle diameter=0.020 inches (0.0508 cm)

The cross-sectional area of this nozzle (“A_{nozzle}”) is then πD²/4=2.03×10⁻³ cm².

Consider a typical rate of fluid flow (“Q”) through the pipette tip nozzle of 50 microliters/second, which is equivalent to 0.050 milliliters/sec, which is equivalent to 0.050 cm³/sec.

The average flow velocity (“V_{avg}”) for flow through a circular orifice is defined as Q/A_{nozzle} which is then equal to (0.050 cm³/sec)/(2.03×10⁻³ cm²)=24.6 cm/sec.

The fluid shear stress is defined as τ=μ×(dV_{avg}/dY), where μ=the dynamic viscosity of the fluid and dV_{avg}/dY is the velocity gradient, which is then=(24.6 cm/sec)/(0.0508 cm/2)=968/sec.

μ_{water}=at 20° C.=0.0101 Poise=0.0101 Dyne-sec/cm².

Therefore the fluid shear stress under these conditions=(0.0101 Dyne-sec/cm²)×(968/sec) which is equal to 9.8 Dyne/cm².

Case II: Shear stress for flow through a pipette nozzle diameter=0.005 inches (0.0127 cm)

The nozzle area is then=1.27×10⁻⁴ cm².

Consider the same fluid flow rate=0.050 cm³/sec.

The average flow velocity is then equal to (0.050 cm³/sec)/(1.27×10⁻⁴ cm²)=395 cm/sec.

The velocity gradient is then=(395 cm/sec)/(0.0127 cm/2)=62,200/sec

Consider the same fluid dynamic viscosity=0.0101 Dyne-sec/cm².

Therefore the fluid shear stress under these conditions=(0.0101 Dyne-sec/cm²)×(62,200/sec) which is equal to 628 Dyne/cm².

Consider a fabric mesh containing alternating 10 micron fibers woven in a rectilinear fashion, wherein the each successive fiber in each direction is spaced at 20 micron intervals, thereby creating a “checkerboard” array of 10 micron holes through the fabric mesh (i.e. “pores” in the mesh). This simple, rectilinear mesh design then creates 1 pore for every 3 spaces that contain a fiber. Stated another way, the pore fraction 1/(1+3)=25%. Therefore, a one square centimeter of such a fabric would contain 25×10⁺⁴ 10 micron pores, or a pore density is 25×10⁺⁴ pores/cm².

Case III: Shear stress for flow through a 10 micron mesh placed over a 0.020 inch diameter nozzle

The 0.020 inch or 0.0508 cm diameter has a cross-sectional area=2.03×10³ cm².

Per above, the mesh has 25×10⁺⁴ pores/cm². Therefore, the number of pores covering the nozzle is equal to (25×10⁺⁴ pores/cm²)×(2.03×10⁻³ cm²)=508 pores.

Consider the same fluid flow rate=0.050 c^{m3}/sec. Assuming that this flow is equally distributed across all pores, the flow rate through each individual pore is then (0.050 c^{m3}/sec)/(508 pores) which is equal to 0.00010 c^{m3}/sec.

Since the area of each individual pore is 1.0×10⁻⁶ cm², the average flow velocity is then equal to (0.00010 cm³/sec)/(1.0×10⁻⁶ cm²)=100 cm/sec.

The velocity gradient is then=(100 cm/sec)/(0.001 cm/2)=200,000/sec

Consider the same fluid dynamic viscosity=0.0101 Dyne-sec/cm².

Therefore the fluid shear stress under these conditions=(0.0101 Dyne-sec/cm²)×(200,000/see) which is equal to 2,020 Dyne/cm².

Case IV: Shear stress for flow through a 10 micron mesh placed over a 0.010 inch diameter nozzle

The cross-sectional area of nozzle=0.508×10⁻³ cm².

The number of pores covering the nozzle is equal to (25×10⁺⁴ pores/cm²)×(0.508×10⁻³ cm²)=127 pores.

Consider the same fluid flow rate=0.050 cm³/sec. The flow rate through each individual pore is then (0.050 cm³/sec)/(127 pores) which is equal to 0.00040 cm³/sec.

The average flow velocity is then equal to (0.00040 cm³/sec)/(1.0×10⁻⁶ cm²)=400 cm/sec.

The velocity gradient is then=(400 cm/sec)/(0.001 cm/2)=800,000/sec

Consider the same fluid dynamic viscosity=0.0101 Dyne-sec/cm².

Therefore the fluid shear stress under these conditions=(0.0101 Dyne-sec/cm)×(800,000/sec) which is equal to 8080 Dyne/cm².

Referring to “Hydrodynamic Shear Breakage of DNA” by Bowman et.al., Biopolymers 11, 2601 (1972), specifically FIG. 4, shear stress required to initiate DNA breakage, we see a threshold shear stress requirement (i.e. slope) of 23 Dyne-sec/cm² for each 1.0 micro-gram of DNA/milliliter of solution, or 23 Dyne-ml/cm²-microgram.

Typical polymerase chain reaction concentrations contain 100 ng of DNA per μl of solution, which is equivalent to 100 μg per ml of solution. Therefore, the maximum allowable fluid shear stress to avoid initiating DNA shear would be:

(23 Dyne-ml/cm²-microgram)×(100 micrograms/ml)=2,300 Dyne/cm².

Likewise, typical DNA concentrations for sequencing, a popular DNA analysis technique, contain approximately 10 ng of DNA per microliter of solution, which is equivalent to 10 micrograms per milliliter of solution. Therefore, the maximum allowable fluid shear stress to avoid initiating DNA shear would be:

(23 Dyne-ml/cm²-microgram)×(10 micrograms/ml)=230 Dyne/cm².

The actual size of pipette tip nozzles and filter pores, sample flow rate, and concentration of DNA in the sample will determine allowable process conditions for each specific application.

A summary of a broad array of pipette tip nozzle diameters and mesh pore sizes for a wide range of flow rates is listed in FIG. 48 for two popular DNA concentrations (i.e. PCR and Sanger Sequencing).

5.8 Surface Area of Adsorption Material

In another embodiment, the surface area of any support containing the adsorption material or agents (e.g., particle or film) is preferably between 0.001 and 0.500 square meters per 50 μl of sample volume. This aspect of the present invention creates a high adsorbing material surface-area-to-fluid-volume ratio that provides the ability of a small device package size to adsorb an adequate proportion of materials from a 50 μl sample volume. This provides a high design degree of safety with regard to the area required to reliably remove a specific amount of material from solution.

5.9 Immobilization of Adsorption Materials

In another embodiment, the adsorption particles (or materials) are immobilized onto a support (or multiple supports), wherein the size of the support is greater than the diameter of the pipette tip nozzle orifice. In this embodiment, the relatively small particles are prevented from passing out the relatively larger pipette nozzle or plugging the nozzle and preventing fluid flow through the nozzle by virtue of their attachment to the larger support.

In this particular embodiment, the minimum chord length of the support(s) is greater than the maximum dimension across the pipette tip nozzle and/or the maximum effective size of the openings of the retaining means at either the distal and/or proximal ends of the pipette tip. One example of such a support is the elongated film shown in FIG. 15a. In this particular example, the width of the support (show in FIG. 15a as “Minimum Support Width”) is wider than the diameter of the pipette orifice.

5.10 Packaging of Adsorption Materials

In another embodiment, the adsorption material is placed into a porous package, analogous to a “tea bag,” which is placed into pipette tip, as shown in FIGS. 28a-b. In this embodiment, the package consists of a porous film, such as a biologically inert polypropylene mesh or similar material, wherein the effective pore size of the mesh is smaller than the size of the particles retained within the package. The porous container can be placed into and subsequently removed, one or more times, from the housing containing the materials to be removed from the sample.

In another embodiment, particles containing a single adsorption material are placed into a package (containing two or more particles such as shown in FIG. 28b) and two or more packages placed into the pipette tip.

In another embodiment, a package containing a mix of particles wherein each particle either contains a different adsorption material or multiple adsorption materials are contained on a single particle, and one or more small packages placed into the pipette tip.

In another embodiment, two or more packages are placed into the pipette tip wherein each package contains a single adsorption material, different from the adsorption material contained in other packages placed in the pipette tip.

The prepackaging of adsorption materials simplifies the loading of reagents during the pipette tip manufacturing process, it can allow significant variation in the volume and type of adsorption material(s) that are loaded into the pipette tip. The particle-bearing package(s) may be retained within the pipette tip using any of the approaches described herein above for retention of particles. Preferably, when one package is used, the shape of the package, such as its taper as shown by angle θ in FIG. 28a, is similar to that of the inside of the pipette tip, or alternately that the package is pliable so as to be able to conform to the inside of the pipette tip with the application of a small package insertion force, so as to ensure maximum flow of sample fluid through and not around the package.

In another aspect of this embodiment, a minimum package diameter “D” is used that is similar in size to the inside diameter of the pipette nozzle so as to minimize the volume of fluid between the package and the pipette nozzle (i.e., the sample “dead volume”). Since different size pipette tips require different “D” dimensions, this requirement is properly specified by limiting this “dead volume” to preferably no larger than 10% of the volume of sample fluid aspirated into the pipette tip for purification.

In another embodiment, and in lieu of a porous material to form a package, the particles can be bonded together into a single volume of particles, using for example, biologically inert adhesives, similar to that shown in FIG. 28a, but without the need for the porous walled container to contain the particles. In this embodiment, which is illustrated in FIG. 28c, the single bonded volume of particles is placed into the pipette tip.

In another embodiment, two or more bonded volumes of particles, wherein each particle contains a single, identical adsorption material are placed into the pipette tip. This particular configuration is illustrated in FIG. 28d.

In another embodiment, one or more bonded volumes of particles are placed into the pipette tip, wherein each bonded volume contains two or more different adsorption materials. According to this aspect, multiple particles can be used in each bonded volume. For example, each particle can contain different adsorption materials or t multiple adsorption materials can be contained on each single particle. This particular configuration is illustrated in FIG. 28f.

In another embodiment, two or more bonded volumes of particles are placed into the pipette tip, wherein each bonded volume contains a single adsorption material different from the adsorption material contained in the other bonded volume(s) of particles placed in the pipette tip. This particular configuration is illustrated in FIG. 28e.

These embodiments provide the following advantages. Loading of reagents during the pipette tip manufacturing process is simplified. Significant variety in the volume and/or type of adsorption material(s) that are loaded into the pipette tip is possible. The bonded volume or volumes of particles may be retained within the pipette tip in the manner described herein for particles.

Preferably, when one bonded volume is used, the shape of the bonded volume, such as its taper as shown by angle θ in analogous FIG. 28a, is similar to that of the inside of the pipette tip, or alternately that the bonded volume is pliable so as to be able to conform to the inside of the pipette tip with the application of a small insertion force so as to ensure maximum flow of sample fluid through and not around the package. FIG. 28a also shows that a minimum bonded volume diameter “D” can be used that is similar in size to the inside diameter of the pipette nozzle so as to minimize the volume of fluid between the distal end of the bonded volume of particles and the pipette nozzle. Since different size pipette tips require different “D” dimensions, this requirement is properly specified by limiting this “dead volume” to preferably no larger than 10% of the volume of sample fluid aspirated into the pipette tip for purification.

### 5.11 Positioning of Adsorption Materials

In another embodiment, the adsorption material(s) are attached to the inside surface of the pipette tip (FIG. 29). In another embodiment, the pipette tip is coated with two or more different adsorption materials.

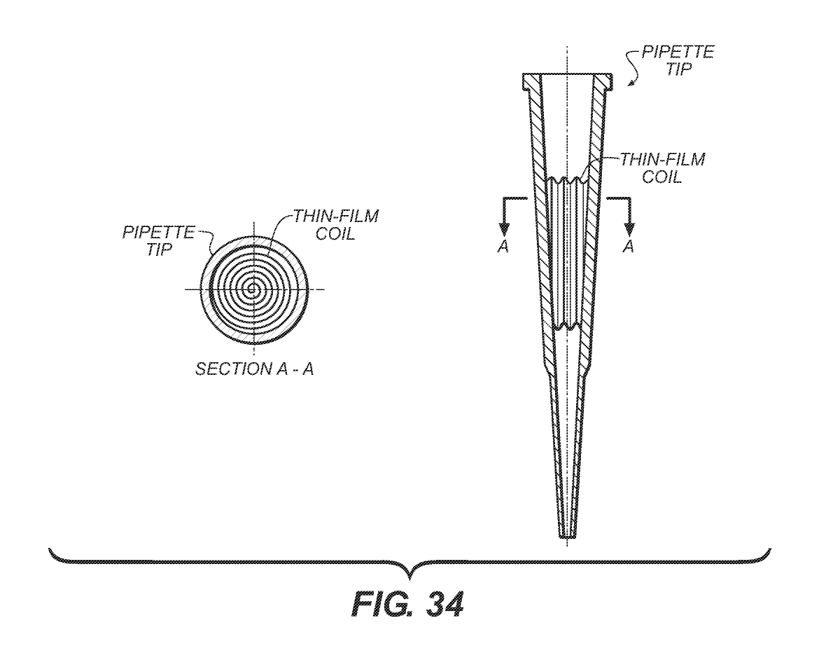

In another embodiment, the adsorption material or agents are attached to the surface of a film which is placed inside the pipette tip (FIG. 34). Preferably, the film is rolled up into a column (e.g., a spiral) so as to provide a large film surface area. In this embodiment, the diameter of the coil is preferably larger than the diameter of the pipette nozzle so as to contain the film within the pipette tip. The film may also be retained within the pipette tip in the manner described earlier for retaining particles or packages or bonded volumes containing particles. A further refinement of this particular embodiment is the use of a porous film to further increase surface area and therefore the volume of adsorption material contained by the film, thereby further enhancing the volume of materials that may be removed by a given size of film.

In another embodiment, the adsorption material or agents are attached to the surface of a film which is compressed into a small volume or multiple volumes and placed inside the pipette tip (FIG. 30). In this embodiment, the compressed film is prevented from passing through the distal end of the pipette tip by utilizing a compressed film volume or volumes that are larger than the maximum diameter of the pipette nozzle. The compressed film or films are retained in the proximal end of the pipette tip as described herein.

In another embodiment, the adsorption material or agents are attached to multiple features within the pipette tip that serve to increase wettable surface area, such as multiple, internal, axial running ribs, such as those shown in FIG. 31. In this embodiment, the ribs may be an integral part of the pipette tip (i.e., a molded feature of the pipette tip) or a pre-formed member that is placed inside the pipette tip before molding (i.e., insert molding), or after molding. In the latter embodiment, the pre-formed insert is retained within the distal end of the pipette tip by utilizing a pre-formed insert whose cross-sectional area is larger than the diameter of the pipette tip nozzle. The insert is retained at the proximal end using the various methods described herein and/or by the use of adhesives, or various welding techniques such as ultrasonic or laser welding.

In another embodiment, a dissolvable porous media is used in the housing, such as a test tube or PCR reaction vessel. In this embodiment, the dissolvable media can either be added to a vessel already containing the sample to be purified whereby it absorbs the sample fluid to be purified using capillary flow into the media, purify the sample and subsequently dissolves, or contained in the vessel prior to adding the sample fluid whereby the addition of the sample fluid results in absorption of the fluid by the porous media through capillary flow into the media, purification of the sample fluid and subsequent dissolution of the particle volume. In either case, the capillary flow induced fluid uptake eliminates the need for specialized fluid handling equipment during fluid uptake. The particles containing impurities are separated from the sample containing the purified DNA as described herein, including filtration, sedimentation, centrifugation, magnetic separation or other separation technologies. In the case of magnetic separation, the dissolvable porous media would be comprised of particles that had magnetic properties. It is to be understood that the dissolvable volume(s) of porous media may contain one adsorption material or multiple adsorption materials.

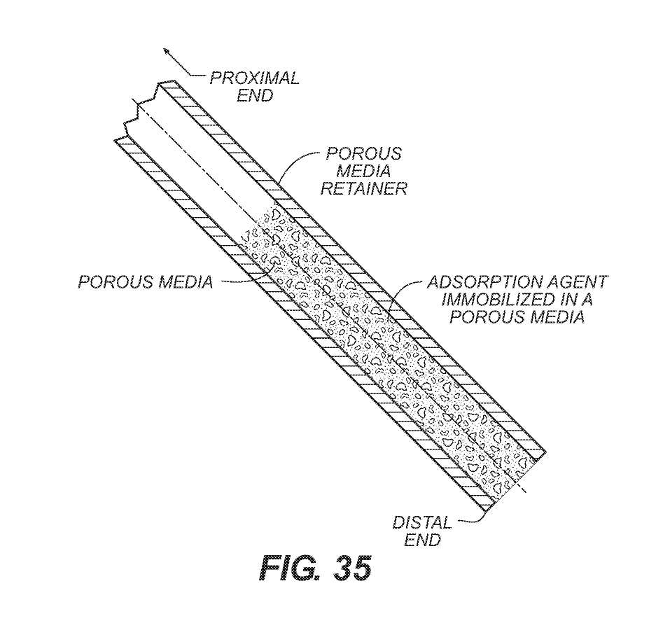

Adsorption material or agents can be attached to a high surface area material, such as for example the internal and/or external surfaces of a volume or volumes of a porous media, such as for example, a “sponge” consisting of a biologically inert matrix of hydrophilic (i.e., wettable) material, which is placed inside the pipette tip. Suitable wettable materials are well known in the art. For example, the wettability of an inherently low wetting material such as polypropylene can be increased by activating the surface with an oxygen plasma and then treating with PEG. Likewise, the wettability of a silica materials can be increased by reacting it with an oligoethylenglycol silane. This embodiment includes the advantage of large coated surface area per unit sample volume. The sample fluid to be purified is brought into contact with the distal end of the porous media retainer (FIG. 35). Capillary pressure and/or differential fluidic pressure created across the porous media volume by, for example, the application of fluid pressure or mechanically decompressing the porous media, forces fluid to pass into the porous media where it comes into contact with the adsorption material. The fluid is forced from the porous media by, for example, physically compressing the porous media retainer, applying differential fluidic pressure across the porous media, or by contacting the distal end of the porous media retainer with a material of higher surface energy (i.e., higher capillary force) than the porous media within the porous media retainer. The use of capillary pressure to transfer fluid into the pipette tip eliminates the need for devices such as pipettor to apply fluid pressure during fluid intake (i.e., aspiration). The porous media is retained at the proximal end by as described herein,

The fluid sample purification system is not limited to the use of compression and/or differential fluidic pressure to extract fluid from the porous media. Additional embodiments to perform this function can include, but are not limited to the following fluid extraction methods known in the art: (1) Exposing the pipette tip and porous media to centrifugal force (e.g., spinning the pipette tip in a centrifuge) to force sample fluid from the media; (2) Removing the porous media from the pipette tip and applying compression and/or differential fluidic pressure to force sample fluid from the media.

In another embodiment, the adsorption materials are incorporated into the pipette tip using a combination of the above supports, such as particles and/or packages of particles and/or coated inner pipette surfaces and/or films.

In another embodiment, the adsorption material or agents are attached to the surfaces (external and/or internal) of a volume or volumes of porous media comprised of bonded particles, with the further feature that porous media can dissolve, releasing the bonded particles into solution, enabling the particles to, for example, settle in the solution through sedimentation, all occurring after the sample fluid to be purified is placed into contact with the particles. In this embodiment, the particles are bonded together using suitable methods known in the art, for example, through the use of: sugars or other soluble biocompatible adhesives; electrostatic attraction, such as by coating at least a portion of the particle surface with positive and negative charge materials; or a dissolvable package to contain the particles; such as a porous gelatin material similar to that used for encapsulating drugs.

This embodiment provides the additional advantages of using capillary flow into the porous media to facilitate fluid uptake, the dissolution of the bonded particles and porous media, and simplifying the recovery of the purified sample from the particles. When using this particle configuration within a pipette tip, the dissolvable particles containing the materials to be removed from solution are retained within the pipette tip as described herein. The dissolvable volume(s) of porous media may contain the multiple combinations of adsorption materials.

### 5.12 Agitation of Particles

In another embodiment, the pipette tip or reaction vessels described above containing the loosely packed particles is agitated, such as by the use of shaking and/or vibrating, to enhance the movement of the particles in the sample solution and thereby further enhance their likelihood of encountering materials in the sample to be removed, thereby further increasing their adsorption capacity (i.e., the volume or mass of material removed from the sample). The use of fluid agitation generally creates high shear forces between the sample fluid and the adsorption surfaces, and thereby minimizes the thickness of the mass diffusion boundary layer at the adsorption surfaces, which helps to increase the rate of diffusion of the adsorbable material to the immobilized adsorption material, thereby minimizing the time required to adsorb materials in the sample. The use of fluid agitation also increases the likelihood that a particle containing an adsorption material will encounter a material to be adsorbed, thereby further enhancing the effectiveness of the adsorption process. In a specific embodiment, a mixing process is used wherein the sample fluid to be purified or a portion of it is alternately aspirated into and out of the pipette tip two or more times.

In another embodiment, the particles in the pipette tip or reaction vessel are agitated by the use of magnetic stirring, to enhance the movement of the particles in the sample solution and thereby further enhance their likelihood of encountering adsorbable materials in the sample, such as DNA, thereby further increasing their adsorption effectiveness and capacity, and reducing the time required to do so. In this embodiment the particles would possess magnetic properties.

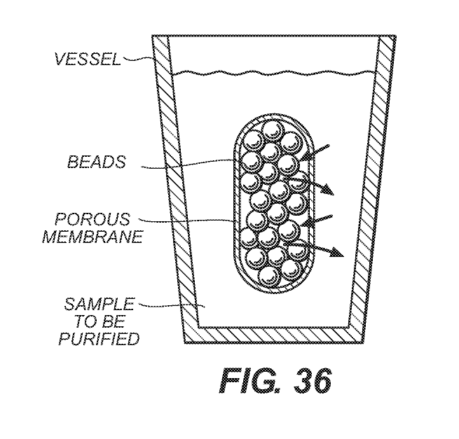

In another embodiment, a particle-containing package, such as that shown in FIG. 36, is inserted in the sample fluid and agitated, such as by the use of stirring or repeated insertion and removal, to enhance the movement of fluid through the container in the sample solution, thereby enhancing the likelihood of encountering materials to be adsorbed, such as DNA, thereby increasing its adsorption efficiency and reducing the time required to do so.

### 5.13 Thermal Deactivation and Denaturation of Sample

In another embodiment, the housing (e.g., pipette tip or other sample-bearing vessel) is heated so as to denature nucleic acids (e.g., DNA) in a fluid sample and to allow the adsorption of single-stranded nucleic acids (DNA) occurring as a result of denaturing double stranded nucleic acid molecules (e.g., ds-DNA). In a specific embodiment, denaturation is conducted in the presence of loosely packed functionalized particles or particles of adsorption material.

In one particular refinement of this embodiment, the sample fluid is heated to a temperature that preferentially denatures double-stranded DNA, such as hybridized DNA pairs containing base-pair mismatches, and as a result of these mismatches, lowers denaturation temperatures relative to other DNA sequences. The mismatched DNA adsorbed by the particles is then selectively removed from the solution.

In another embodiment, the housing (e.g., pipette tip or other sample-bearing vessel) are heated so as to deactivate other impurities, such as the enzyme used to facilitate the PCR reaction, Taq polymerase.

### 5.14 Target Adsorption

The fluid sample purification system can comprise a low target (e.g., DNA) adsorbing functional pipette tip (or column) and/or low target DNA adsorbing filter material, combined with adsorption materials that simultaneously adsorb other (undesirable) DNA materials.

The performance of any nucleic acid fluid sample purification system can be assessed, in part, by the amount of target nucleic acid that is returned by the purification process, often referred to as “target yield.”

Many existing products separate biological materials by initially adsorbing both impurities (e.g., unincorporated PCR primers and nucleotides) and desired target (e.g., PCR amplicons), and then exploiting the differential solubility of impurities and desired target to sequentially re-suspend and remove each material. The fluid sample purification system operates very differently, by adsorbing only the impurities and simultaneously repelling the desired target, all in a single step, which is a considerable advantage. Therefore, unlike existing purification products, the fluid sample contacting materials used in the fluid sample purification system need to resist and not encourage undesirable adsorption of the desired targets (e.g., ds-DNA), since no additional steps are available to elute any target materials that may bind to any fluid-contactable materials used in the fluid sample purification system. By contrast, this condition is not required by existing purification products. It is also difficult to achieve in practice because nucleic acids tend to bind to some extent to many surfaces.

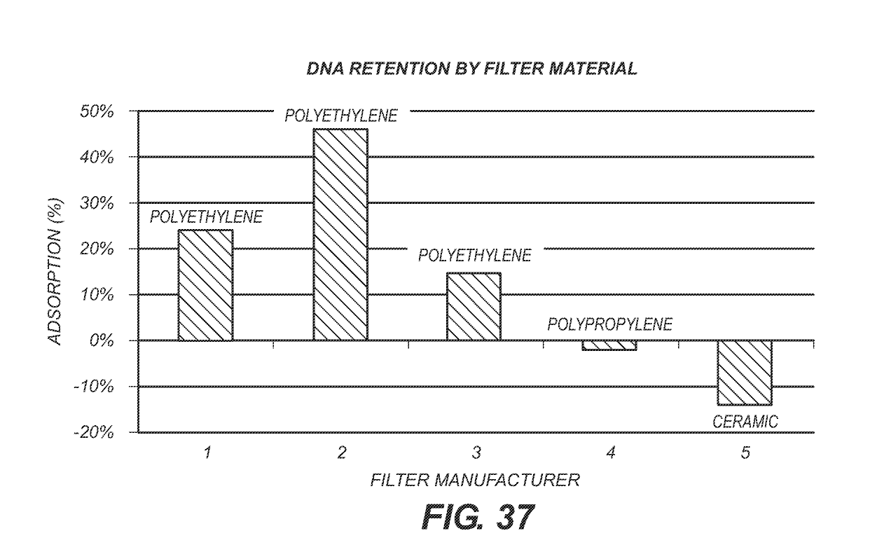

FIG. 37 shows that conventional filter materials can adsorb significant quantities of DNA. By contrast, another aspect of the fluid sample purification system includes the simultaneous use of fluid sample contacting materials, including filter materials that resist adsorption of desired targets. The specific choice of materials will vary by application and specific target type. For applications where the target is DNA, materials with neutral or negative surface charge at pHs of 6.0 to 9.0 have very low affinity for adsorption of DNA. Silica is one such material that has very low affinity for adsorption of DNA. The filters are preferably free of any addenda used during the filter manufacturing process, since many organic and inorganic substituents can also bind DNA. FIG. 37 shows such a filter, a ceramic filter comprising silica clad alumina.

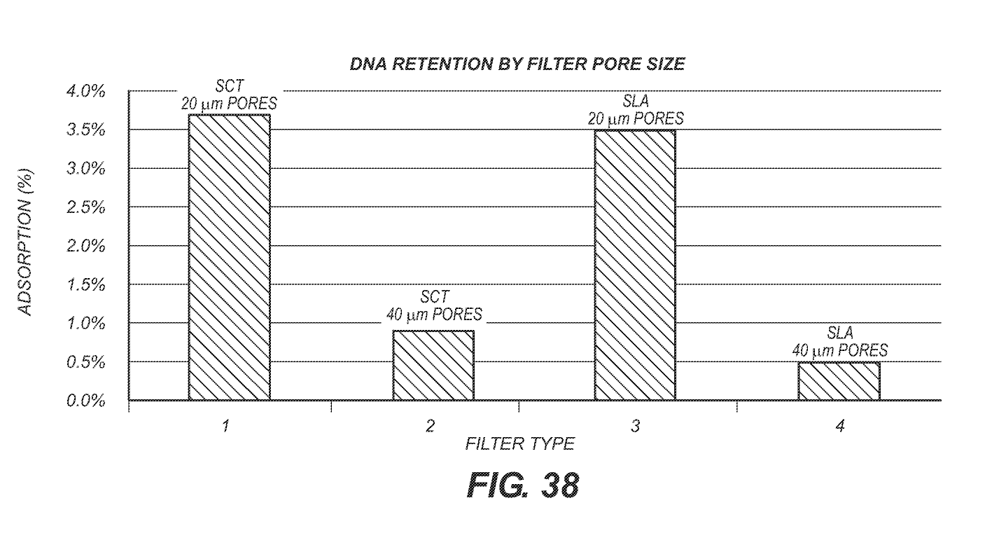

Another aspect of the fluid sample purification system and methods is the ability to minimize target adsorption (e.g., DNA adsorption) by minimizing filter area, which is a function of filter pore size. FIG. 38 shows the amount of DNA adsorbed by two different filter materials, SLA and SCT (both silica clad alumina) and two different filter pore sizes (20 μm and 40 μm). It is clear from this figure that larger pore size filters result in less DNA adsorption, regardless of the type of filter material, owing to the larger surface area associated with the smaller 20 μm pore size filters. Accordingly, optimal filter pore size for DNA adsorption purposes is then 40 μm or larger, depending upon other system constraints (e.g., adsorption particle retention).

Another aspect of the fluid sample purification system and methods is the ability to concentrate samples. In one embodiment, the filter material of the distal retainer concentrates the sample by pulling water out of solution. FIG. 37 shows that the ceramic filter used in this embodiment of the fluid sample purification system increases sample DNA concentration by approximately 14% (i.e., a negative adsorption value of 14%) This is achieved by using a filter material that (chemically) removes water from the sample while not removing the desired target (e.g., DNA), thereby increasing the concentration of the remaining target. Such filter materials that remove water are well known in the art. Thus, the fluid sample purification system employs pipette filter designs capable of achieving low DNA adsorption or even DNA concentration, while simultaneously satisfying all previous system-level and filter performance requirements. Also, such pipette filters can be used in conjunction with adsorption materials that are specifically designed to simultaneously adsorb other (undesirable) materials.

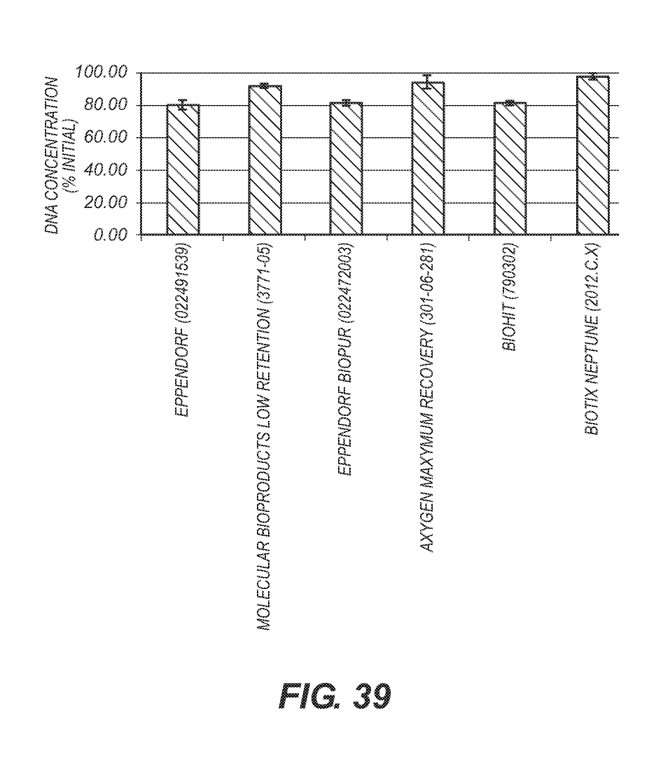

FIG. 39 shows that standard laboratory pipette tips and commonly used pipette filter can adsorb significant quantities of DNA. Therefore, another aspect of the fluid sample purification system is the use of pipette tips that use functionalized particles to adsorb lesser quantities of DNA.

Two general methods for reducing DNA adsorption by surfaces can be used. One method is to treat the surface of the DNA-bearing solution with a negatively charged poly-electrolyte, such as PAA. Table 3 shows that increasing PAA exposure of the pipette tip surface which contacts the DNA-bearing solution reduces DNA adsorption by up to 34%.

A second method is to create an exceptionally smooth surface on the pipette tip surface which contacts the DNA-bearing solution. Table 3 shows that reducing surface roughness from 0.7 micro-inches RMS to 1.4 micro-inches RMS reduces DNA adsorption by 84% or more, relative to pipette tip surfaces with surface roughness of 2.0 to 5.5 micro-inches RMS. The combination of a smooth and chemically treated surface can reasonably be expected to further reduce DNA adsorption.

TABLE 3
Effect of Pipette Tip Surface Finish
and Coating on DNA Adsorption

| Pipette Tip # | Surface Finish | DNA Adsorption |
|---|---|---|
| #1 | 2.0-5.5 micro-inches | 12.8% |
| #2 | 0.7-1.4 micro-inches | 2.1% |
| #3 | 0.7-1.2 micro-inches | 1.0% |

| Pipette Tip # | Surface Coating | DNA Adsorption |
|---|---|---|
| #1 | None (Control) | 12.8% |
| #2 | PAA (1 min) | 11.8% |
| #3 | PAA (5 min) | 9.8% |
| #4 | PAA (10 min) | 8.8% |
| #5 | PAA (15 min) | 8.5% |

### 5.15 Filter Intrusion and Extrusion Pressure

Filter intrusion pressure is the pressure required to wet the filter or flow duct so that fluid flow through the filter or duct can initiate. Filter extrusion pressure is the pressure required to unwet the filter or flow duct and force out any fluid sample that may he contained in the voids of the filter or duct. Both can be features of the fluid sample purification system. Maximizing the amount of purified fluid sample returned to the user by minimizing the volume of sample retained within the fluid sample purification system is an advantageous feature, particularly when processing very small sample volumes.

Given the very low pressures developed by standard pipettors, a low intrusion pressure is desirable to help ensure reliable initiation of sample flow through a filter or duct and into the pipette tip. The wettability of any filter material or duct is a function of its advancing contact angle—high contact angle materials are more difficult to wet than low contact angle materials, and pore or duct size—small pores or duct cross-sections are more difficult to wet than larger ones, owing to their smaller capillary radius (see earlier discussion regarding capillary radius). Pipette tip manufacturers exploit this characteristic when placing aerosol barriers at the proximal end of pipette tips, where the barrier's hydrophobic (i.e., non-wetting) property helps block the flow of liquid out of the pipette tip. Intrusion pressure data for a typically used aerosol barriers is typically several times higher than the maximum pressure that can be developed by pipettors. However, these materials, which often include polymeric materials, represent a poor choice as a distal retainer owing to such high intrusion pressures, which are greater than the pressures developed by commonly used pipettors.

In certain embodiments of the fluid sample purification system, distal retainers with low advancing contact angle materials make more suitable distal retainers, since their intrusion pressures are more similar to that of commonly used pipettors. The lower intrusion pressures provided by the silica filter material relative to typically used polymeric filter materials makes it easier to unwet than many typically used filter materials. Specific methods to enhance the wettability of low-wetting materials have been discussed above.

Thus, the fluid sample purification system uses an optimal pipette filter material that has low intrusion and extrusion pressures (e.g., easy flow initiation and high sample recovery) while simultaneously satisfying the system performance needs of the user.

### 5.16 Reaction Chamber

The fluid sample purification system can comprise a reaction chamber. The reaction chamber can have an open reaction chamber design that does not rigidly contain the reaction particles (contrast with e.g., the design used by the Aspire™ and Zip-Tip™ products). The open reaction chamber design enables adsorption materials to be located at any location within the pipette tip, including attachment to the internal walls of the pipette tip. When the adsorption materials are contained on or within particles, the open reaction chamber design permits the fluid sample and adsorption media to interact over the entire internal volume of the pipette tip. The Aspire™ product has the distinct disadvantage in that the two filters used to contain adsorption materials within an internal void volume do not permit any sample within the pipette tip but outside the void volume to interact with the adsorption materials.

Likewise when a porous matrix is used to contain the adsorption materials (e.g., Zip-Tip™ and Millipore similar to that disclosed in U.S. Pat. No. 6,048,457 by Kopaciewicz), any sample within the pipette tip but outside the porous matrix is not permitted to interact with the adsorption materials.

Therefore, in one embodiment of the fluid sample purification system a larger fluid sample-adsorption material reaction chamber is employed to further improve the effectiveness of fluid sample-adsorption material interaction. Section 6.2 (Example 2) discloses a fluid handling protocol that takes further advantage of such a larger reaction chamber.

The design of the reaction chamber of the fluid sample purification system eliminates failure modes of reaction chamber design that are seen in currently available pipette tips, e.g., in the Aspire™ pipette tip and the pipette tip disclosed in U.S. Pat. No. 6,048,457 by Kopaciewicz, which include the following specific failure modes:

(1) Entrapment of air bubbles within the reaction chamber. The relatively small internal dimensions used in functional pipette tips cause the behavior of fluids within such chambers to be dominated by surface tension (versus gravity) effects. These effects can often trap air bubbles within the chamber. The effects can include increased resistance to fluid flow, reduced interaction with the adsorption materials (e.g., particles) contained within the reaction chamber, and the need for additional operator interaction and fluid aspirate/dispense cycles to remove the bubbles, the latter being very difficult to achieve with automated fluid handling systems.

(2) Creation of a preferred flow path through the reaction chamber, particularly when particles are more confined within the reaction chamber, which prevents all of the particles contained therein from interacting with the sample passing through the chamber.

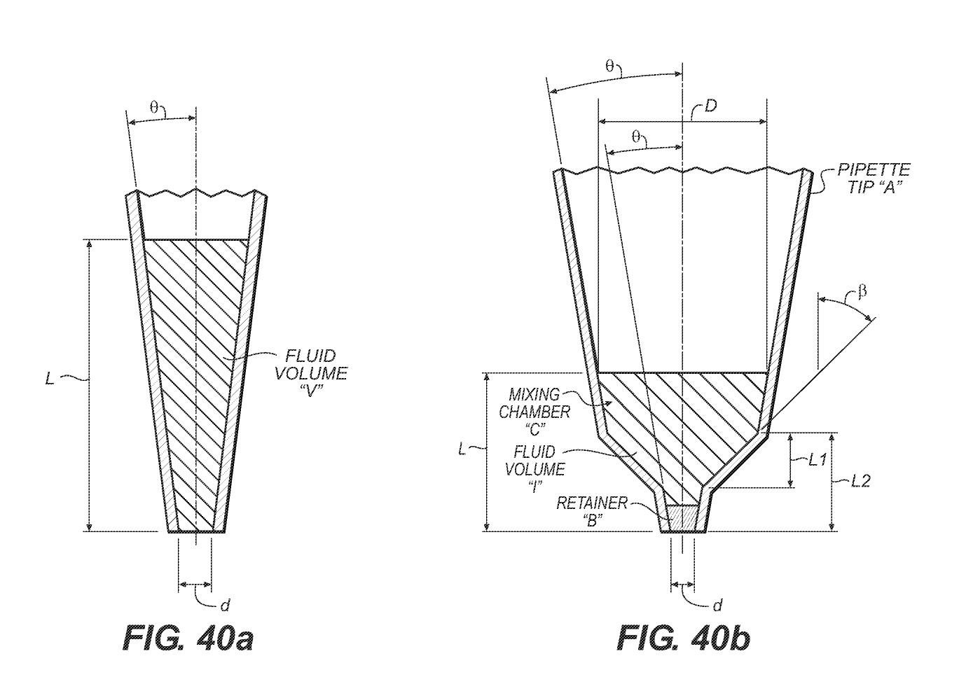

The fluid sample purification system avoids these problems in several ways: (1) by eliminating the confined reaction chamber used by the Aspire and Zip-Tip functional pipette tip designs, in favor of the open reaction chamber design; (2) through the use of hydrophilic particles; (3) a reaction chamber design with low slenderness ratio as shown in FIGS. 40b; and (4) through the use of particles with specific gravity greater than 1.0. The latter aspect of the fluid sample purification system ensures that particles do not float on the surface of the unconstrained fluid, thereby preventing effective sample interaction. The low slenderness ratio reaction chamber is discussed further in Section 6.2 (Example 2).

In summary, the open chamber design of the fluid sample purification system helps to provide the following benefits:

(1) Reduced purification process time owing to more effective interaction of the fluid sample and adsorption materials.

(2) Increased adsorption capacity owing to the ability to immobilize adsorption materials on the relatively large inner surface of the pipette tip.

(3) Increased reliability of operation (e.g., eliminate the potential to trap air within the reaction chamber).

Thus, according to the invention, the performance of functional pipette tip designs that rigidly confine particles in the manner of the Aspire™ and Zip-Tip™ pipette tips can be improved in several ways, including (1) using a smaller number of porous particles within the confined reaction region, and (2) using hydrophilic particles within the confined reaction region.

### 5.17 Fluid Sample Dead Volume

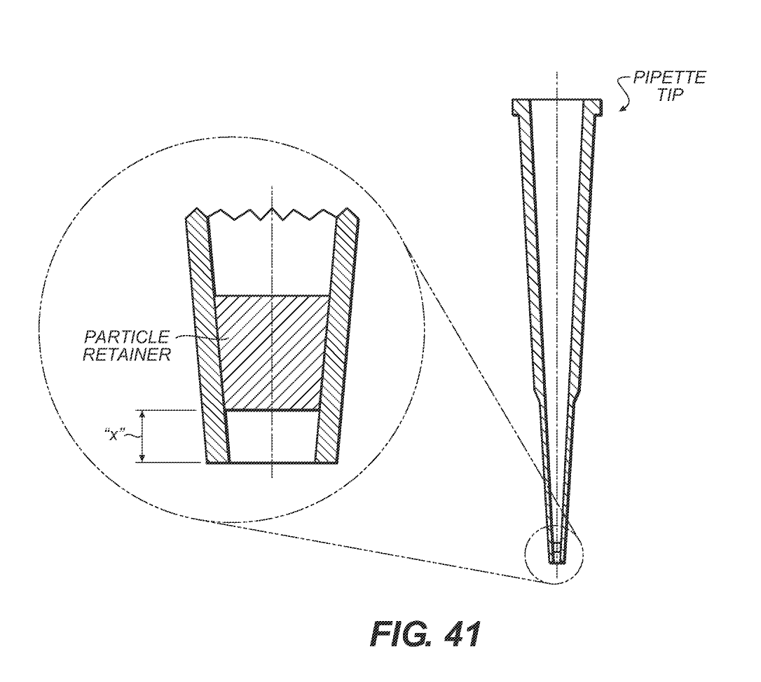

In another embodiment, the distally positioned particle retainer (or filter) minimizes, in the wetted pipette tip, the volume of fluid that is retained between the distal end of the distally positioned particle retainer and the distal end of the pipette tip, as shown by dimension “X” in FIG. 41. Preferably, the particle retainer is positioned axially such that this “dead volume” is no larger than 10% of the total volume of sample fluid aspirated into the pipette tip.

In another aspect of this embodiment, the volume of the fluid retained by the distally positioned particle retainer (such as the void volume of particle retainer comprising a porous matrix as shown in FIG. 20a is preferably no larger than 10% of the total volume of sample fluid aspirated into the pipette tip.

Minimizing the “dead volume” of fluid that is retained in the distal portion of the pipette tip provides several specific advantages. It minimizes the volume of fluid that that does not pass over the particles and therefore is not purified by the pipette tip, thereby minimizing the impurities in the dispensed sample (i.e., improving overall sample quality). It maximizes the volume of fluid that is dispensed from the pipette tip, thereby maximizing the amount of purified sample that is available for subsequent analysis (i.e., improving sample volume recovery).

Any fluid sample contained between the nozzle of the pipette tip and the distal filter is at risk of not being aspirated into the pipette tip and purified by the adsorption materials contained therein. Therefore, it is advantageous to minimize this volume. A preferred embodiment for achieving this objective, while simultaneously satisfying other product design constraints is shown in FIG. 32. Adsorption materials A are packed into the distal end of pipette tip B and are retained within the pipette tip by filters C (optional) and D. Filter C is positioned near the proximal end of the pipette tip where it serves as an aerosol barrier to prevent aspirated fluid from contaminating the pipettor (not shown). Filter D is positioned near the distal end of the pipette tip to minimize the volume of fluid contained between the pipette tip nozzle and (distal) end of filter D. To further minimize sample dead volume, the body of the pipette tip is rapidly tapered over transition region E, after distal Filter D, to a very small diameter sample acquisition feature F. The use of a very small sample acquisition nozzle combined with a short transition region serves to minimize the volume of contained between the distal filter and pipette tip nozzle G.

The pipette tip design of the fluid sample purification system therefore minimizes sample dead volume while enabling the use of a distal retainer (or filter) with relatively large cross-sectional area, while simultaneously satisfying previous requirements.

### 5.18 Total Internal Air Volume

Air contained within the pipette tip of the fluid sample purification system is compressible and serves to decrease the maximum pressure or vacuum that can be created by a given pipettor. Minimizing this air volume helps to increase maximum pipettor-applied pressure or vacuum, which in turn helps to create higher fluid flow rates and reduced sample processing time. The specific choice of pipette tip and its associated internal air volume will vary by application. However, the inventors have determined that for best overall system performance, a total internal air volume of no more than 8-10× the sample volume is optimal. This equates to a pipette tip with an internal air volume of no more than approximately 200 μl for a 25 μl sample size. The maximum pipette tip total internal air volume therefore achieves high fluid flow rates and minimal sample processing time.

### 5.19 Fluid Extraction

The use of a surface coating and/or micro-capillary ducts in a pipette tip can be used to prevent a mass of small particles from trapping sample fluid within the particles contained in the pipette tip (or other housing), thereby making it easier to extract the sample fluid from the particles and pipette tip.

Wetted particles create a capillary pressure at the fluid-air interface. This capillary pressure is defined as P_{c}=2γ/r where γ is the surface tension of the fluid and r is the radius of the fluid surface at the fluid-air interface. The use of a hydrophilic material, whose surface energy is greater than that of the particles, disrupts these smaller fluid radii at the fluid-air interface and draws liquid from the wetted volume of particles. This embodiment provides the advantage of preventing aggregate volumes of small particles from trapping fluid by virtue of their many small fluid radii at the fluid-air interface.

In another aspect of this embodiment, the interior of the pipette tip is coated with a high energy, highly wettable coating to drain fluid from the wetted mass of particles. Specific methods to enhance the wettability of low-wetting materials are described above.

### 5.20 Fluid Sample Purification Methods

Methods for isolating a molecule of interest in a fluid sample using the fluid sample purification system are provided. In one embodiment, the method can comprise:
a. providing a fluid sample to be tested for the presence of the molecule of interest;
b. providing a fluid sample purification system;
c. drawing the fluid sample into the fluid sample purification system;
d. contacting adsorption material or a plurality of functionalized particles with the fluid sample; and
e. aspirating and dispensing the fluid sample using an aspirating/dispensing cycle and fluid flow rate that maintains a consistent and consistently high rate of fluid flow, thereby expelling the fluid sample from the fluid sample purification system.

The method can further comprise the step of recovering the molecule of interest from the expelled fluid sample. In various embodiments, a yield of at least 70% or at least 95% of the molecule of interest can be obtained.

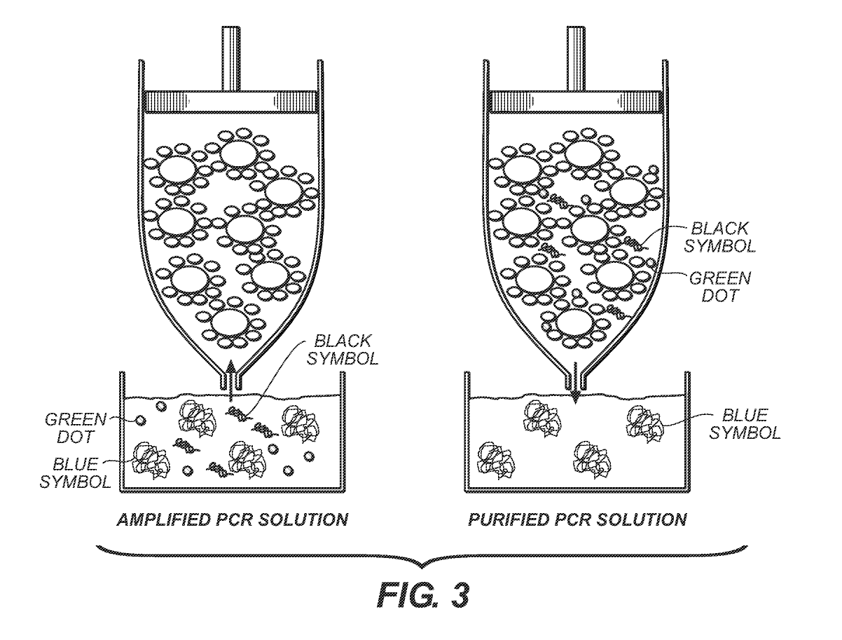

FIG. 3 shows a schematic of one embodiment of the fluid sample purification method in which DNA is being purified. The left sketch depicts aspiration of target DNA (“blue symbol”), unamplified PCR primers (“black symbols”) and unincorporated nucleotides (“green dots”) in PCR solution into a chamber containing adsorption particles. The expelled solution in the right hand sketch contains target DNA free of impurities (e.g., primers and nucleotides).

The fluid sample purification methods of the invention can comprise fluid sample aspirate and dispense protocols that can be used with the fluid sample purification system. As used here, the word “protocol” describes how the fluid sample purification system is operated by an end-user, such as, for example, how a pipettor may be programmed to manipulate a fluid sample.

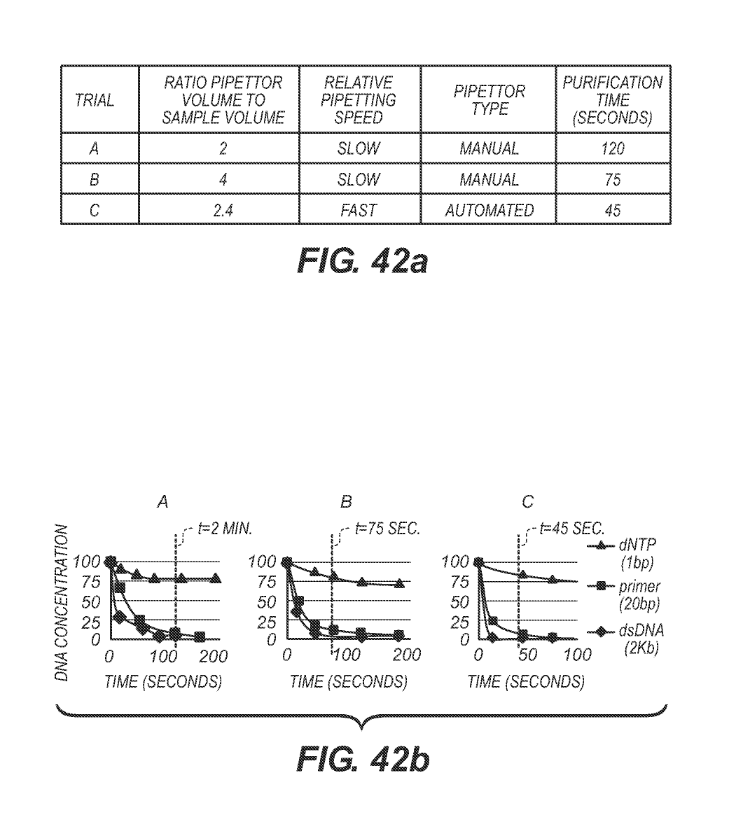

A preferred method for using the fluid sample purification system comprises using a fluid aspiration and dispensing protocol wherein the interaction between the adsorption materials and materials to be removed from the aspirated sample is improved by “over-aspirating” and “over-dispensing” the fluid sample. Over-aspiration involves aspirating fluid sample into the pipette tip followed by an additional bolus of air. This method has the effect of creating greater agitation within the pipette tip owing in part, to the additional motion of the fluid sample, as well as the general “flattening” of the fluid column at a higher position and hence larger internal diameter within the pipette tip, which has the further effect of more effectively distributing the particles within the fluid. FIGS. 42a-b show a base condition wherein a total volume equal to 2× the sample volume is aspirated and dispensed (i.e., aspiration/dispensing of one part sample and one part air). This results in a purification reaction time of 120 seconds. Increasing the total aspirate/dispense volume to 4× the sample volume (i.e., aspiration/dispensing of one part sample and three parts air) results in a purification reaction time of 75 seconds—a reduction of 37%. In one embodiment, a 25 μl sample and 75 μl of air are aspirated into and dispensed from a 200 μl pipette tip.

Another aspect of using an over-aspirate and over-dispense protocol is the ability to significantly reduce sample processing time while increasing process reliability. More specifically, the over-aspiration protocol involves the aspiration of a small volume of liquid followed by the aspiration of a larger volume of air to achieve the greater level of sample agitation previously demonstrated. The fact that air passes through the distal filter at a much greater flow rate provides several benefits:

(1) Reduced purification time. The faster rate of air flow significantly increases the flow rate of the fluid previously aspirated into the pipette tip, thereby reducing aspiration/dispensing cycle time and further enhancing sample agitation, relative to the situation wherein the aspirated/dispensed volume is exclusively liquid.

(2) More consistent tip-to-tip aspirate and dispense volumes. The functional pipette tip assembly has much lower resistance to air flow during the portion of the air aspiration/dispensing process, owing to the lower viscosity of air relative to liquid. Since the higher liquid flow resistance is encountered over only a portion of and not the entire aspiration/dispensing cycle, tip-to-top variation in liquid fill volume is minimized.

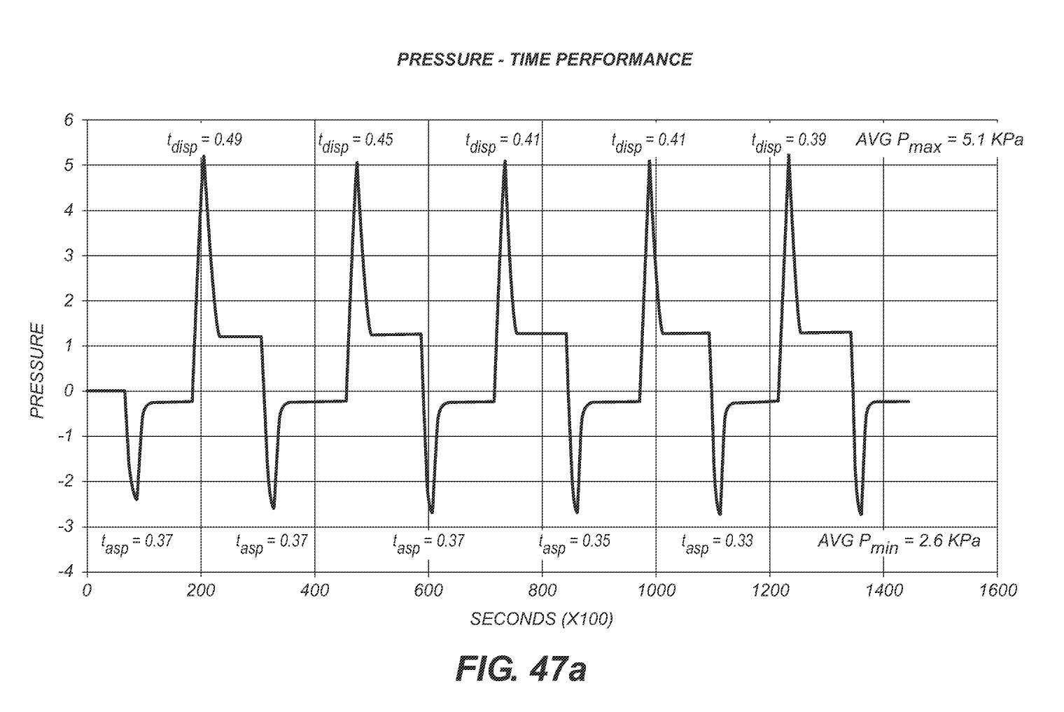

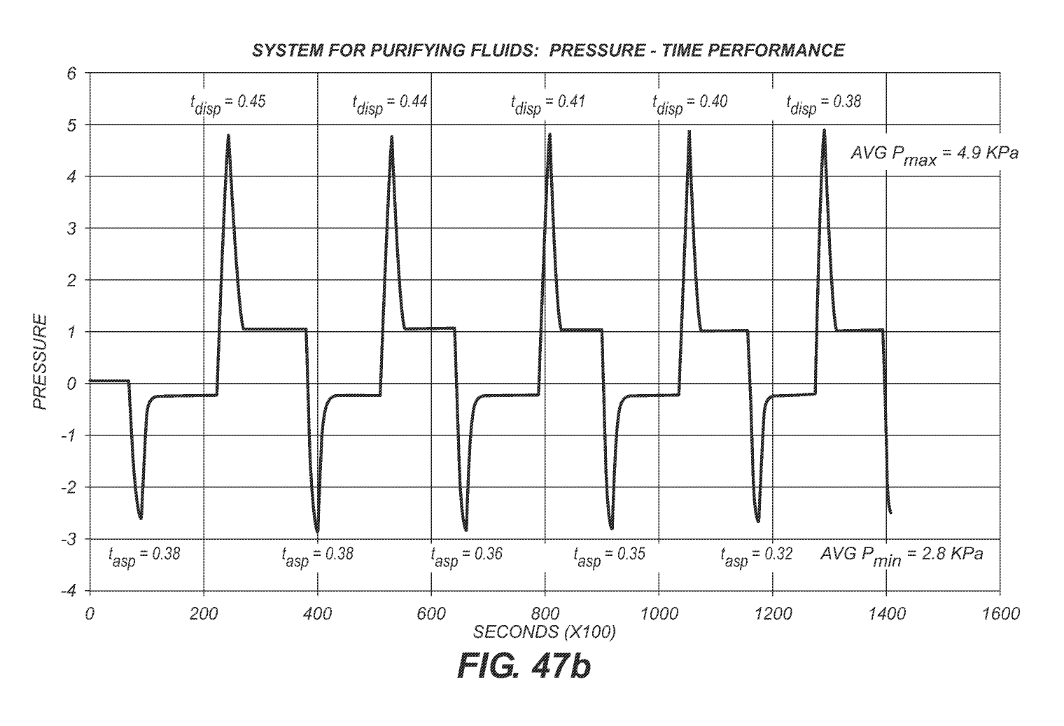

The purification method can comprise the step of using multiple fluid sample aspiration and dispensing cycles to help ensure that the retainer material does not become plugged with adsorption particles. Forcing fluid sample through the retainer in alternating directions (i.e., aspirate and dispense) helps ensure that any particles that may be drawn into or accumulate against the face of the retainer during the dispense cycle can be forced out and re-suspended in solution during the subsequent aspirate cycle. FIGS. 17a-d shows that the dispensing pressure within a particle filled pipette tip remains constant over many successive aspiration and dispensing cycles, even though dispense pressures increase momentarily as particles accumulate against the face of the retainer. This self-cleaning action is particularly noticeable in FIGS. 47a-b, which illustrate very consistent fluid aspirate and dispense pressures over multiple aspiration and dispense cycles. This self-cleaning action also helps to preserve a high rate of flow through the retainer as well as help to ensure that all particles are available to interact with the sample and help to remove impurities.

The purification method can comprise the step of using over-aspiration and over-dispensing steps to achieve the following sample purification improvements: First, the use of over-aspiration is used to force any sample dead volume into the pipette tip to ensure that this volume interacts with the contained adsorption particles and is purified, thereby ensuring a high level of sample quality. Second, the use of over-dispensing involves dispensing an additional volume of air to ensure that the over-aspirated sample is dispensed from the pipette tip.

Thus, the purification method permits a high rate of flow through a filter and as a result, reduced sample processing times, avoids plugging of particle retainers, and helps to ensure a high level of sample quality by minimizing the effects of sample dead volume. With respect to fluid sample aspirate and dispense rates, the time required for functionalized particles to bind to impurities in the sample solution depends in part upon the level of fluid agitation, which depends in part upon the rate at which fluid sample is aspirated into and dispensed from the pipette tip. FIGS. 42a-b show that increasing pipetting speed from slow to fast (e.g., a pipettor that has a rate of (piston) displacement less than approximately 400 μl/sec to greater than approximately 500 μl/sec), results in a purification reaction time of 45 second, which is a further reduction in purification reaction time of 40% from the previous cycle time of 75 seconds, despite that the over-aspirate/over-dispense ration was reduced from the previous optimum of 4× to 2.4×.

Thus, embodiments of the method for fluid sample purification provide optimum fluid aspirate and dispense rates for achieving the level of agitation required to achieve minimum sample purification times. More specifically, the minimum fluid flow rate is high enough to effectively agitate the functionalized particles within the fluid sample, while low enough to be achievable with the level of pressure typically applied by existing pipettors.

### 5.21 Advantages of the Fluid Sample Purification System and Methods

In addition to satisfying the basic product performance requirements for high sample quality and yield, speed (less than one minute), and reduced costs (especially labor costs), the fluid sample purification system is easy to dispose, frees up bench space by not requiring a centrifuge, magnetic extractor or vacuum manifold as is typical of most other cleanup protocols, and does not require additional solvents or other reagents as is required in most other cleanup protocols. Furthermore, the reliability of the purification process is improved significantly by eliminating the need for multiple operator interactions.

The optimization and component integration involved in the fluid sample purification system has enabled the development of a fluid sample purification system with performance benefits that far exceed the performance of existing purification products, and would not have been possible without such a system approach.

In addition to providing high sample quality and yield, ease of use, speed and reduced costs (especially labor costs), the fluid sample purification system is easy to dispose, frees up bench space by not requiring additional capital equipment such as a centrifuge, magnetic extractor or vacuum manifold as is typical of most other cleanup protocols, and does not require additional solvents or other reagents as is required in most other cleanup protocols. Furthermore, the reliability of the purification process is improved significantly by eliminating the need for multiple operator interactions.

Pipette tips are used in practically all laboratories for transferring liquid samples from one vessel to another. Since customers already use pipette tips with existing purification products, the equipment (i.e., pipettor) and training required to use the fluid sample purification system in pipette tip format (i.e., comprising a pipette tip housing) is already in place. There is no need to add extra equipment to the laboratory regardless of laboratory size. Labs that are automated will use a liquid handling robot to clean samples on their established high throughput equipment. Single samples would obviously be purified with a single pipettor—a ubiquitous laboratory tool. Purification columns are equally abundant as receptacles for handling samples to be purified. Users of this product design see that the product design is a seamless fit with their current laboratory operations. The switching cost for the laboratory to integrate the technology is zero. The ease of implementing this product is a distinct advantage to the customer.

The fluid sample purification system can be integrated seamlessly with existing manual and automated laboratory methods, including specifically DNA purification methods.

The fluid sample purification system and methods improve reliability of the purification process, owing in part to the minimization or in some cases elimination of operator interactions and the potential for operator error which can adversely affect the quality of the subsequent nucleic acid analysis, or in the case of limited sample size, prevent a subsequent analysis from taking place.

Rapid purification time are possible (approximately 30-60 seconds versus 15-30 minutes or more for competitors) depending upon application, owing to:

(a) The ability of the fluid sample purification system to incorporate into a single pipette tip or columns all of processes necessary to purify a sample, including specifically purification of nucleic acids;

(b) The use of binding materials whereby the binding reaction coefficient enables nearly instantaneous binding of the impurities (see Appendix 1);

(c) The use of embodiments of the fluid sample purification system that create high fluid agitation thereby minimizing the time required for impurities in a fluid sample to diffuse to an adsorber (i.e., create a shorter impurity-particle diffusion path lengths);

(d) Improved impurity adsorption capacity and sample quality, i.e., the ability, for example, to remove a greater portion of impurities owing to an excess of adsorbing surface area, combined with the use of agitation to increase the likelihood that a greater percentage of impurities will contact an adsorber, thereby increasing the effectiveness of the purification process. Sample quality is also positively affected in the fluid sample purification system by eliminating the chemicals used by other purification processes which adversely affect subsequent analysis of the sample;

(e) Improved target yield. The fluid sample purification system is fundamentally different from existing purification methods, since it adsorbs only impurities (e.g., ss-DNA) and not the target (double-stranded) DNA. Therefore, the fluid sample purification system is not affected by the efficiency with which target is released from the adsorber. Target yield is also positively affected by the use of low DNA adsorbing surfaces.

(f) Improved sample volume recovery. The fluid sample purification system uses far fewer fluid contactable consumables, each of which can retain fluid sample. Sample recovery is further enhanced by the use of low fluid retention particles.

(g) Low total procedure cost. The fluid sample purification system enables significant reduction in sample (e.g., nucleic acid) purification costs owing in part to the following factors;

(h) Reduction in labor requirements enabled by the pipette tip, in which are integrate, many (or all) of the functions preferably used to purify samples, thereby minimizing or eliminating the labor required to produce a purified sample.

(i) Reduction in the number of disposables required to perform a purification, as well as the fluid sample purification systems use of an inexpensive disposable pipette or column. The elimination of the requirements for additional instrumentation or services required (e.g., centrifuges or vacuum lines).

(j) The elimination of the requirements for additional reagents;

(k) Small package size. The present provides complete purification capability in a very small package size owing in part to the reduction in the number of disposables required to perform a purification. This feature helps to minimize product storage requirements;

(l) Reduced waste owing to the fact that the purification function is completely contained within a single pipette tip or filtration column which does not require additional consumables of reagents; and

(m) Ease of use. The fluid sample purification system simplifies the sample purification procedure by integrating into a single pipette tip or filtration column all of the functions required to purify a target-bearing sample, the user needs only to load the pipette tip onto a manual or automated fluid pipettor. The act of aspirating and dispensing the sample is sufficient to accomplish purification. Existing purification processes use pipettors only to transfer, not purify samples, and contain considerably more mechanical and chemical processes.

It will be apparent to the skilled artisan that numerous embodiments of the systems and methods presented herein can be envisioned based upon various combinations of adsorption materials, housing designs (e.g., pipette tip versus column versus vessel), system components (e.g., retainer and seal configurations), fluid sample handling protocols, etc. The following examples are offered by way of illustration and not by way of limitation.

### 6. EXAMPLES

### 6.1 Example 1

### Preferred Embodiment of Fluid Sample Purification System

This example demonstrates a preferred embodiment of the fluid sample purification system. The system comprises a distal particle retainer that has effective particle retention, minimizes resistance to fluid flow, has consistent tip-to-tip flow resistance, has faster sample processing time, minimizes retention of sample fluid, has improved product manufacturability, has reduced product manufacturing cost, and has improved sample quality.

This example describes an improved pipette filter design that can be used in the fluid sample purification system. Specific features of the filter include very low fluid flow resistance, consistent fluid flow resistance, uniform pore size, low sample retention and low cost. Unlike prior art fluid sample purification systems, the embodiment of the fluid sample purification system with this filter design has features within the pipette tip, rather than a separate filter, to provide the filtration function.

In FIG. 25, pipette tip ‘A’ includes insert ‘B of thickness ‘t’ positioned within pipette tip ‘A’ by insert positioning tabs ‘C’ creating fluid flow conduits ‘D’ of width ‘E’. Cross-section A-A through pipette tip ‘A’ illustrates fluid flow through the axially-running fluid flow conduits ‘D’ formed by the space between pipette tip ‘A’ and insert ‘B’. Conduit width ‘E’ is precisely determined by the recess in insert ‘B’, such as for example, by the injection mold that precisely forms this feature of the part during the insert injection molding process. The width ‘E’ of flow conduits ‘D’ are chosen to minimize fluid flow resistance, while in one application preventing larger objects (e.g. particles) from passing into or out of pipette tip ‘A’.

In FIG. 26, pipette tip ‘A’ includes insert ‘B of thickness ‘t’ positioned within the pipette tip by insert positioning tabs ‘C’ creating fluid flow conduits ‘D’ of width ‘E’. Unlike the embodiment of the fluid sample purification system shown in FIG. 25, the flow conduits are created in the inner wall of pipette tip ‘A’. Cross-section A-A through pipette tip ‘A’ illustrates fluid flow through the axially-running fluid flow conduits ‘D’ formed into the wall of pipette tip ‘A’. Conduit width is precisely determined by the “core” of the pipette tip injection molds that precisely forms the internal surfaces of the pipette tip ‘A’ during the pipette tip injection molding process. As before, the width ‘E’ of flow conduits ‘D’ are chosen to minimize fluid flow resistance, while in one application preventing larger objects (e.g. particles) from passing into or out of pipette tip ‘A’.

This filter design has the ability to effectively retain particles within the pipette tip. A conduit dimension of, for example, 0.002 inches corresponds to 50 micro-meters, which would effectively prevent the passage of particles 50 micro-meters or larger. Particles are frequently in various ways when processing fluids. Depending upon the size, shape, specific gravity and level of agitation, particles from 50 to 500 micro-meters or larger are often used to process fluids. Accordingly, a preferred conduit width ‘E’ is 0.002 inches to 0.020 inches (i.e. 50 to 500 microns) with 0.002 to 0.006 (50 to 150 microns being most optimal.

This filter design has the ability to minimize flow resistance. It is well known that larger flow cross-sections provide less resistance to fluid flow. The annular-shaped conduits created between pipette tip ‘A’ and insert ‘B’ in this embodiment of the fluid sample purification system provide a large flow-through area and therefore low resistance to fluid flow, relative to, for example, a porous medium such as a porous frit. In FIG. 25 or FIG. 26, a pipette tip inside diameter of 0.060 inches, an insert with four 0.010 inch wide positioning tabs that create a conduit width of 0.004 inches provides a total flow cross-sectional area that is 0.000554 square inches. By comparison, the flow cross-section of a 0.020 inch diameter pipette tip nozzle is 0.000314 square inches. In this specific example, the cross-sectional area of the flow conduit is 1.76 times larger than that of the unrestricted pipette nozzle. Likewise, the flow conduit of this embodiment is also much larger than the effective flow cross-sectional area of any porous frit or membrane of the same size. Furthermore, the straight-through flow path design of this embodiment creates less resistance to fluid flow than an equivalent thickness of porous material, such as a porous frit. The design minimizes the thickness ‘t’ of flow restrictor ‘B’ and therefore minimizes flow resistance.

It is well known that resistance to flow through a conduit is proportional to the length of the conduit, with shorter lengths producing less resistance to flow. Accordingly in the present invention, the length of conduit, ‘t’, is preferably less than the diameter of the conduit (e.g. internal diameter of a pipette where the insert is placed), with a length between 10% and 50% of the size of the insert being most optimal.

The filter design enables faster sample processing time. The higher fluid aspirate and dispense times enabled by this embodiment of the fluid sample purification system can reduce sample processing time for at least two reasons: First, the shorter time required to move fluid into and out of the pipette tip due to reduced resistance to fluid flow. Second, of the greater level of agitation caused by the higher fluid flow rates during sample aspiration and dispensing cycles, which serves to enhance sample-particle reaction kinetics (e.g. the adsorption of materials included in the sample).

This embodiment of the fluid sample purification system minimizes the thickness ‘t’ of flow restrictor ‘B’ and minimizes sample fluid retention. The relatively narrow width ‘E’ of flow conduit ‘D’ can create high capillary pressures at the points where fluid enters and exits flow conduit ‘D’. In some cases, the pressure applied to pipette tip ‘A’ (i.e. such as by a pipettor) may not be sufficient to force fluid from conduit ‘D’. Minimizing the thickness ‘t’ of insert ‘B’ minimizes the volume of liquid that would be retained in flow conduit ‘D’ in these cases.

The precisely controlled cross-sectional area of the flow conduits, combined with the precisely controlled thickness of the restrictor plate, create a very consistent tip-to-tip flow resistance. This performance characteristic is particularly important in applications where multiple pipette tips are attached to a single pipettor with a common aspirate/dispense pressure manifold, wherein the same aspirate/dispense pressure is applied to every pipette tip. Pipette tips that do not contain any internal filters have negligible resistance to fluid flow and are generally known to fill at the same rate and to the same level. However, as resistance to flow increases, any variation in resistance to fluid flow (e.g. a more restrictive pipette tip filter) will cause the affected pipette tip to receive more or less fluid. In the worst case, one pipette tip could receive little or no fluid while another is filled to capacity, possibly contaminating the pipettor. This embodiment avoids this problem by both minimizing overall flow resistance and precisely controlling those factors that affect flow resistance, including the length and cross-sectional area of the flow conduit.

This embodiment has improved product manufacturability. There is no need to precisely locate insert ‘B’ to properly control fluid flow into and out of the pipette tip. Unlike many filter designs wherein the filtering element must he registered (i.e., sealed) against the pipette tip, the present invention relies upon the easily controlled inner pipette tip and insert diameters to secure the insert into the pipette tip and create the precisely controlled flow conduit. The assembly so created can then perform properly regardless of the vertical position of the insert within the pipette tip. Furthermore, unlike porous frits which can be compressed to an indeterminate amount during insertion, thereby creating uncontrollable changes in fluid flow resistance, compression of the insert used in the present invention is not only much less likely (i.e., the insert is produced from a solid material with very high compression strength) but more importantly would not affect fluid flow resistance since flow occurs around and not through the insert. Accordingly, proper insertion of the insert is much less sensitive to variation in insertion force during the assembly process.

This embodiment has reduced product manufacturing cost. The features of the flow conduits are determined during the fabrication of the insert (FIG. 25) or core of the pipette tip mold (FIG. 26), which once completed, eliminates the need for expensive, resistive frits or fragile membranes.

This embodiment has reduced likelihood of damaging materials in the sample. It is well known that passing certain materials, such as DNA, through small flow cross-sections can damage the DNA. The straight-through flow path design avoids this potential problem by eliminating the relatively much more torturous flow paths associated with materials such as porous frits. This aspect also enables much higher fluid flow rates, relative to those that would be possible through materials such as porous frits.

FIGS. 47a-b are graphs of fluid aspirate and dispense pressure—time profiles showing the very low resistance to fluid flow and short cycle times achieved by this embodiment.

Performance of Fluid Filter

The performance of an embodiment of the fluid sample purification system in pipette tip format with flow conduits is shown in FIGS. 47a-b, which was compared with performance of a prior art pipette tip in FIGS. 12a-c. Internal pressure versus time performance was plotted for the fluid sample purification system and for the prior art pipette tip using two samples in each tip.

The fluid sample purification system in pipette tip format with flow conduits aspirates and dispenses fluid much faster than the prior art pipette tip. The flow conduits enable a much shorter sample processing time for two reasons: (1) less time to manipulate the sample fluid and (2) more rapid impurity binding kinetics due to the higher level of agitation achieved, hence shorter sample processing time as compared with the prior art pipette tip to achieve the same level of sample purification.

The fluid sample purification system in pipette tip format with flow conduits has very consistent aspirate/dispense cycle-to-cycle and tip-to-tip performance, in particular, shorter fluid aspirate and dispense times and lower aspirate and dispense pressures.

The fluid sample purification system in pipette tip format with flow conduits effectively eliminates undesirable “conditioning time,” i.e., the time it takes for successive sample aspirate and dispense cycles to reach a minimum time. By way of comparison, the aspirate time for the prior art pipette tip begins at 5+ seconds and reaches 2.6 seconds after three cycles, whereas the aspirate time for the fluid sample purification system begins at 0.37-0.38 seconds and reaches 0.36-0.37 seconds after the same number of cycles.

### 6.2 Example 2

### Fluid Sample Purification System Comprising Pipette Tip with Mixing (Reaction) Chamber

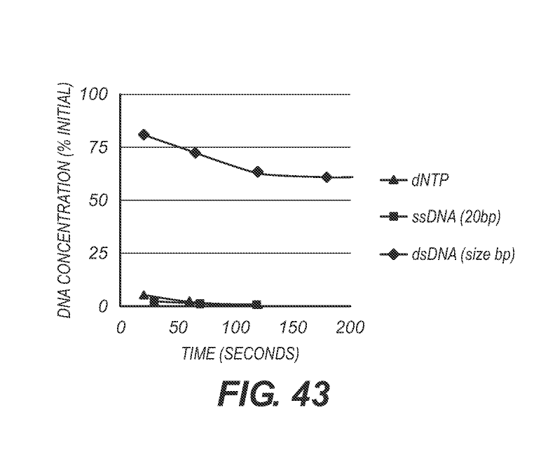

This example describes an additional embodiment of the fluid sample purification system that comprises a pipette, tip that has a different fluid mixing (or reaction) chamber design than described above. The mixing (reaction) chamber has a low slenderness ratio, which design permits more effective interaction between the aspirated/dispensed sample and adsorption materials. FIG. 40b shows pipette tip A containing loosely packed particles (not shown but similar to FIG. 2) and distal particle retainer B designed with a mixing chamber C with low height L. (FIG. 40a shows a typical prior art pipette tip configuration.) There exists a transition region which transitions nozzle diameter d to internal pipette diameter D in a very short distance. Preferably, angle β is approximately thirty degrees so as to help ensure uniform transition of fluid flow from the pipette nozzle to the larger inside diameter of the pipette tip (e.g., no stagnant eddies). Owing to its small diameter d, aspirated fluid will enter the pipette tip at a velocity that is higher than the velocity with which fluid moves further along within chamber C. Owing to the short transition region and lower height of the fluid within chamber C, the higher velocity fluid entering the pipette tip is able to more effectively mix the sample within the pipette tip. FIG. 43 shows that the time required to remove (i.e., adsorb) undesirable impurities from solution is approximately 20-30 seconds. By comparison, FIGS. 42a-b show that under similar conditions the time required to remove undesirable impurities to a similar level required in excess 0145 seconds.

FIG. 40b shows that the dimensions of this pipette tip design create an internal slenderness ratio (i.e., the ratio of height to diameter) that is preferably approximately 1.0. By comparison, FIG. 40a shows a more typical design pipette tip with typical pipette tip dimensions. These dimensions create a slenderness ratio of approximately 8-10 or more. Given the same nozzle diameter d as shown in FIG. 40b, fluid enters the pipette tip at the same velocity, for a given applied vacuum. However, for any given volume of fluid sample aspirated into the pipette tip, a much higher column of fluid is found within the pipette tip owing to the smaller internal cross-sectional area of the more slender pipette tip.

This smaller cross-sectional area creates three problems: First, it enables greater viscous interaction between the aspirated fluid and the internal wall of the pipette tip, thereby reducing the fluid velocity and subsequent mixing within the fluid. Second, it creates a much longer path length for the higher velocity fluid entering the nozzle to travel to interact with fluid at the upper end of the fluid column (i.e., the fluid first aspirated into the pipette tip), thereby further reducing bulk mixing of the fluid. Third, it enables higher density particles within the fluid to settle a further distance from the fluid at the upper end of the fluid column, thereby minimizing the interaction between the particles and fluid, which also results in less effective bulk mixing of the fluid within the pipette tip.

The effect of such an optimally designed reaction chamber is shown in FIG. 43, which shows very rapid impurity binding kinetics and short purification process time.

### 6.3 Example 3

### Fluid Sample Purification System Comprising Pipette Tip with Minimum Internal Air Volume

This example describes an additional embodiment of the fluid sample purification system that comprises a pipette tip with an internally positioned piston to minimize internal volume and create higher fluid sample aspiration and dispense pressures.

This embodiment of the fluid sample purification system comprises a pipette tip and a piston that provides minimum air volume within the pipette tip. Since the air contained within the pipette tip is a compressible fluid, the more air there is in the tip, the less the pressure or vacuum can be generated within the pipette tip for a given displacement of the pipettor's piston. Since the difference in pressure between the inside and outside of the pipette tip is the motive force for moving liquid into or out of the pipette tip, a smaller pressure or vacuum differences result in a lower sample flow rates. Sample flow rate is further reduced by the resistance to flow caused by the adsorption media and filters located in the pipette tip. This lower rate of flow increases the time required to process a particular sample, potentially to unacceptable levels, owing to reduced fluid agitation (i.e., longer time required to adsorb impurities) and additional aspirate/dispense cycle time that is required.

By minimizing the volume of air within the pipette tip and pipettor, a given displacement of the pipettor piston creates a higher pressure difference within the pipette tip and as a result, much higher fluid sample flow rate and shorter time to process a particular sample. Minimizing internal air volume also enables higher internal pressure within the pipette tip with which to more effectively force fluid from within the void volume between the particles, thereby improving sample volume recovery.

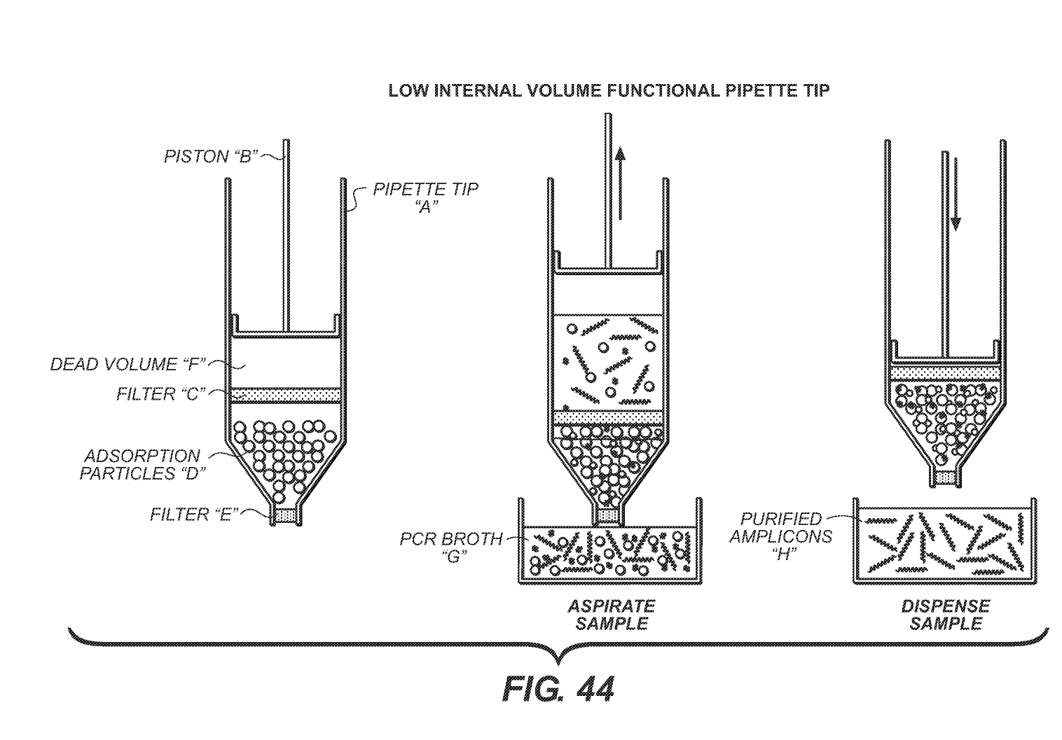

FIG. 44 shows that pipette tip A consists of piston B and filters C and E which confine adsorption material D. Piston B is positioned within the pipette tip, versus within the pipettor, to create a minimum “dead volume” which consists of the air trapped within filters C and E and adsorption material D. Preferably, this dead volume is no more than 100% of the sample volume. As before, a user aspirates into the pipette tip the fluid sample to be processed. The impurities are adsorbed onto the adsorption materials contained within the pipette tip. Dispensing the solution from the pipette tip yields the desired purified sample in a single step.

### 6.4 Example 4

### Fluid Sample Purification System Comprising a Filtration Column

This example describes an additional embodiment of the fluid sample purification system that comprises a filtration column instead of a pipette tip. The adsorption material or agents are either attached to the membrane within the column (i.e., the surface of the membrane contains the adsorption chemistry) or placed upon particles that are either contained in a filtration column prior to adding the fluid sample, or added to the sample before, during or after the sample is added to the filtration column, whereby they are allowed to attach to impurities to be removed from the sample solution.

This embodiment of the fluid sample purification system comprises particles that simultaneously adsorb impurities and repel desired target and a filtration column. The combination of a filtration column, filter and particle size is such that the desired target (e.g., ds-DNA) is allowed to pass through the filter while the impurities are either adsorbed by the membrane or attached to adsorptive particles that are retained by the filter.

According to the fluid sample purification method described in this example, samples are rapidly purified in a single step without the need for additional chemicals.

The fluid sample purification method described in this example also permits applied forces, such as centrifugation, applied pressure or applied vacuum to be made sufficiently high to efficiently remove fluid sample from the column as well as from the void spaces within the particles and filters.

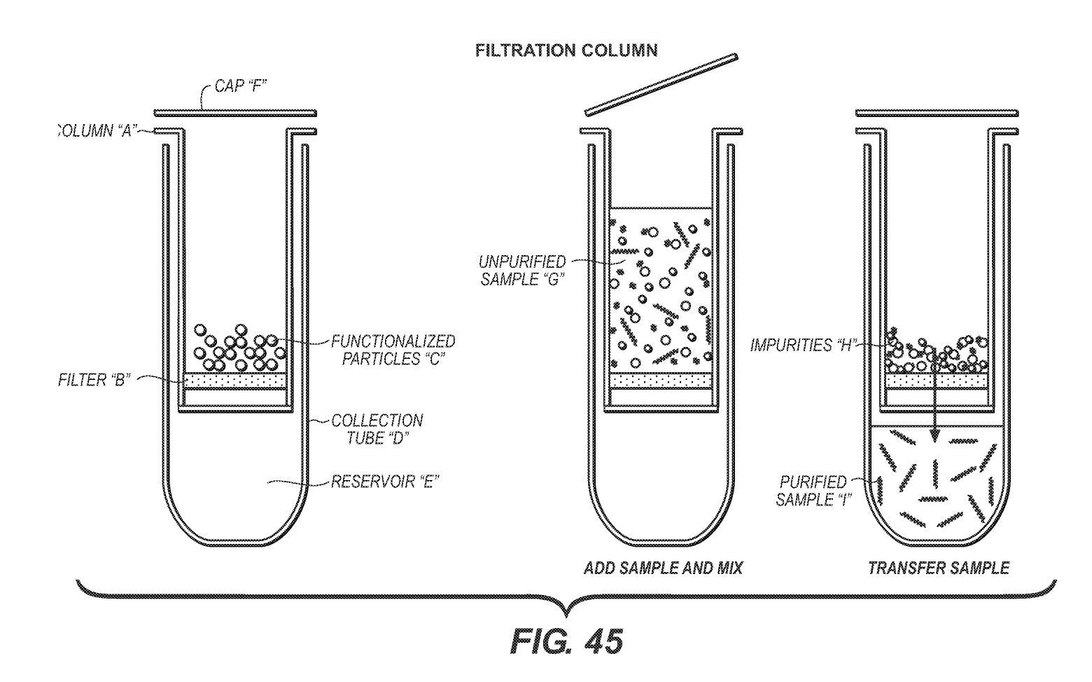

The fluid sample purification method described in this example can be readily used in filtration column format, in which the column contains particles comprising an adsorption material that preferentially adsorbs different types of biomolecules, e.g., nucleic acid, such as single- versus double-stranded DNA, or short versus long double-stranded DNA. FIG. 45 shows a filtration vessel that contains column A to receive the sample. At the base of the column A is filter B. The characteristics of filter B are chosen such that they can be functionalized with differential adsorption materials, or do not pass the particles C and therefore any materials bound to the particles, while at the same time allowing passage of desired materials not bound to the particles. Column A is located in collection tube D which has fluid sample collection reservoir E and removable cap F to secure column A in position and prevent loss of fluid sample. Once the membrane contacts the sample fluid, or once the particles have been mixed with the unpurified sample G, the purified sample I is transferred to the collection reservoir E in collection tube D by any one or more of several methods, including centrifugation, the application of positive pressure to the top side of filter B to “push” the fluid through the filter, or a vacuum on the bottom side of membrane B to “pull” the fluid through the filter. When necessary, the purified sample I is removed from the collection tube by opening cap F, removing column A with impurities H and accessing the purified sample I.

Prior art particle-filtration based purification processes, by contrast, typically adsorb both the target (e.g., PCR amplified DNA) and impurities (e.g., unincorporated PCR primers and nucleotides) and exploit the differential solubility of the desired target and impurities by using a series of chemical-mechanical processes to selectively and sequentially elute impurities and (ultimately) the desired target before passing either impurities and/or desired target through a filtration membrane.

By contrast, the fluid sample purification system works by adsorbing only the impurities and allowing the desired target (e.g., PCR amplicons) to pass directly and immediately through the filtration column in a single step.

In another embodiment of the fluid sample purification system, the filtration column is integrated into an array of multiple wells. More specifically, multiple columns (A) are integrated into a planar X-Y array (i.e., X number of columns in one direction and Y number of columns in an orthogonal direction), while the same number and X-Y orientation of multiple collection tubes (F) are incorporated into a separate planar array. The array of collection tubes is then placed below the array of columns, thereby creating X times Y number of filtration columns in a single, two-plate assembly.

### 6.5 Example 5

### Fluid Sample Purification System Comprising Column or Tube

This example describes additional embodiments of the fluid sample purification system and method in which samples to be purified do not need to be mixed with reagents and/or other particles prior to purification.

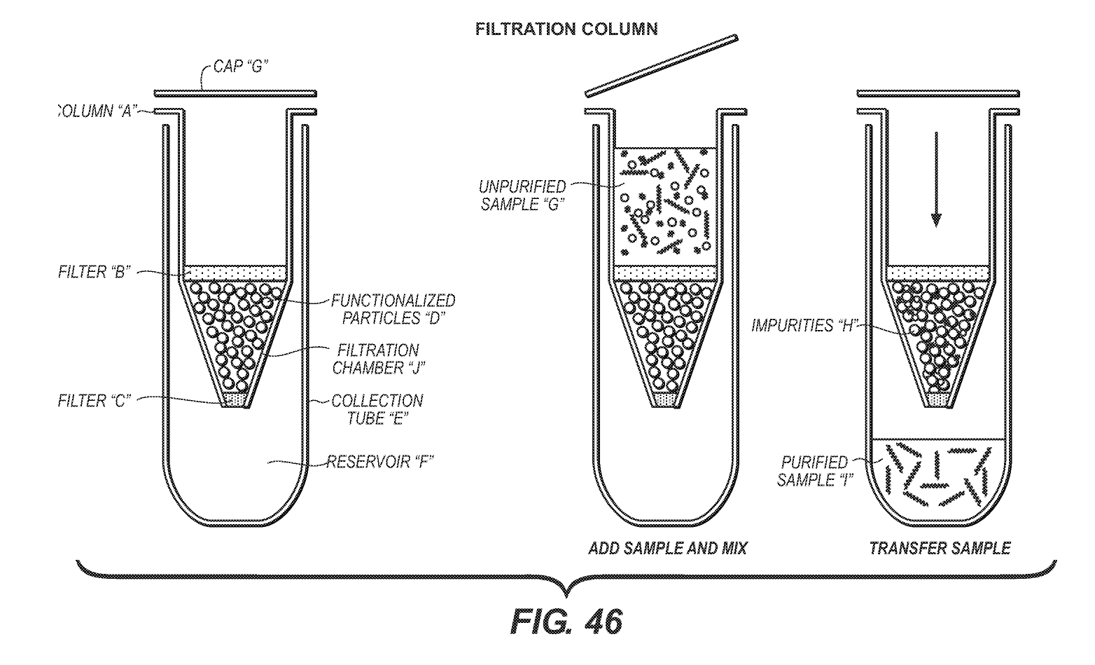

One embodiment of the fluid sample purification system (“column” or “tube” format) uses the column or tube shown in FIG. 46, wherein the filtration column is configured to include a filtration chamber J filled with immobilized adsorption particles D on the downstream side of filter B. Adsorption material or agents are packed into the filtration chamber J and retained in position by filters B (optional) and C. The filtration characteristics of filters B and C are chosen such that they do not pass the particles and therefore any materials bound to them, while allowing passage of desired materials not bound to the particles.

The sample to be purified is added to the column A. However, according to this embodiment, the mixing of the sample and particles is not required. The complete filtration column assembly containing the unpurified sample can be immediately centrifuged or applied to positive pressure or vacuum, to move the sample fluid through the immobilized bed of particles wherein impurities arc adsorbed onto the immobilized particles in filtration chamber J and desired target are simultaneously repelled by the immobilized particles and allowed to pass through. When necessary, the purified sample fluid is removed from the collection reservoir by opening cap G, removing column A and accessing the purified sample I, leaving impurities H in column A.

Prior art purification processes adsorb both the target (e.g., a molecule of interest) and impurities and exploit the differential solubility of the desired target and impurities by using a series of chemical-mechanical processes to selectively and sequentially elute impurities and desired target before passing either impurities and/or desired target through a filtration membrane. By comparison, the fluid sample purification system described in this example works by adsorbing only the impurities and allowing the desired target (e.g., PCR amplicons) to pass through the filtration column in a single step.

In another embodiment of the fluid sample purification system, the filtration column is integrated into an array of multiple wells. More specifically, multiple columns (A) are integrated into a planar X-Y array (i.e., X number of columns in one direction and Y number of columns in an orthogonal direction), while the same number and X-Y orientation of multiple collection tubes (E) are incorporated into a separate planar array. The array of collection tubes is then placed below the array of columns, thereby creating X times Y number of filtration columns in a single, two-plate assembly.

### 6.6 Example 6

### Thermal Denaturation of DNA while in the Presence of Loosely Packed Functional Particles

This example demonstrates an embodiment of the fluid sample purification system in which the pipette tip or filtration column is heated so as to denature the DNA and allow the adsorption of then single-stranded DNA occurring as a result of denaturing the ds-DNA.

The fluid sample is heated to a temperature that also permits preferential denaturization of double-stranded DNA containing base-pair mismatches, which as a result of these mismatches, lowers denaturation temperatures relative to other DNA sequences. The mismatched DNA adsorbed by the particles is then selectively removed from the solution.

Thus, according to this embodiment, the use of thermal denaturation can enable preferential adsorption of specific types of ds-DNA (e.g., hybridized DNA pairs containing base-pair mismatches).

### 6.7 Example 7

### Fluid Sample Purification System Comprising a Pipette Tip

This example describes yet another method for securing a filter into a pipette tip or other similar disposable. In this embodiment, illustrated in FIG. 23, a chisel, preferably self-centering, is forced into the pipette to mechanically form one or more barbs from material from the pipette wall. These barbs then serve to center the filter during the filter insertion process as well as provide mechanical interference to help contain the filter within the pipette tip. This embodiment is particularly advantageous in several respects. First, the small area of contact between the filter and the barbs, relative to the case where the periphery of the filter is in complete contact with the pipette tip, causes a higher level of (localized) contact pressure between the barb and the filter, thereby better securing the filter within the pipette tip. Second, the high (localized) contact pressure is obtained with relatively low filter insertion forces, in a manner analogous to that shown in FIG. 24. This lower force is then less likely to damage the filter, specifically by compressing filter pores and changing its fluid flow resistance. Third, the method eliminates the need for adhesives to secure the filter to the pipette tip, and the potential for these adhesives to interfere with the samples processed by the pipette tip; the filter securing means is the pipette tip material itself.

The invention is not to be limited in scope by the specific embodiments described herein. Indeed, various modifications of the invention in addition to those described herein will become apparent to those skilled in the art from the foregoing description. Such modifications are intended to fall within the scope of the appended claims.

All references cited herein are incorporated herein by reference in their entirety and fir all purposes to the same extent as if each individual publication, patent or patent application was specifically and individually indicated to be incorporated by reference in its entirety for all purposes.

The citation of any publication is for its disclosure prior to the filing date and should not be construed as an admission that the invention is not entitled to antedate such publication by virtue of prior invention.

1-121. (canceled)

122. A fluid sample purification system for isolating a molecule of interest in a fluid sample comprising:
a housing having a distal end with a distal opening adapted for the passage of a fluid and a proximal end with a proximal opening adapted for passage of a fluid;
a distal retainer inside the housing and above the distal opening;
a proximal retainer, wherein the proximal retainer is located:
inside the housing and between the distal retainer and the proximal opening, or adjacent to, in contact with, covering or sealing the proximal opening; and
adsorption material comprising a plurality of functionalized particles, wherein:
the plurality of functionalized particles is inside the housing and confined between the distal retainer and the proximal retainer, and
the functionalized particles of the plurality adsorb undesirable material.

123. The system of claim 122 wherein:
undesirable materials in the fluid sample are adsorbed, and
the molecule of interest or desirable materials in the fluid sample are simultaneously repelled or rejected.

124. The system of claim 122 wherein the functionalized particles of the plurality are differentially functionalized particles.

125. The system of claim 122 wherein the diameters of the functionalized particles are 10-250 μm.

126. The system of claim 122 wherein the plurality of functionalized particles comprise a plurality of diameters, wherein the plurality of diameters is 50-200 μm.

127. The system of claim 122 wherein the functionalized particles are porous.

128. The system of claim 127 wherein the pore sizes of the porous functionalized particles are 5 nm-150 nm.

129. The system of claim 127 wherein the internal surfaces or pore surfaces of the porous functionalized particles are functionalized to adsorb small molecules and the external surfaces are functionalized to inhibit adsorption of larger molecules.

130. The system of claim 122 wherein the functionalized particles comprise:
i) a single type of particle containing one specific impurity adsorption and target rejection chemistry,
ii) a single type of particle containing more than one impurity adsorption and/or target rejection chemistry,
iii) multiple types of particles each containing one impurity adsorption and target rejection chemistry, or
iv) multiple types of particles each containing more than one impurity adsorption and/or target rejection chemistry.

131. The system of claim 122 wherein the adsorption material or the functionalized particles have neutral or negative surface charge at pHs of 6.0 to 9.0.

132. The system of claim 122 wherein the functionalized particles have a specific gravity greater than 1.

133. The system of claim 122 wherein the distal retainer and the proximal retainer are spaced sufficiently far apart to form a void in the presence of the plurality of functionalized particles, and wherein the void is dimensioned so that functionalized particles can travel freely within the void allowing for thorough mixing between the functionalized particles and the fluid sample when the fluid sample is in the void.

134. The system of claim 122 wherein the distal retainer is 0.10 to 1.5 mm thick.

135. The system of claim 122 wherein the distal retainer diameter is 1-3 mm.

136. The system of claim 122 wherein distal retainer effective pore size is 5-250 μm.

137. The system of claim 122 wherein the distal retainer comprises a hydrophilic or wettable material.

138. The system of claim 122 wherein the distal retainer comprises a non-adsorbing material.

139. The system of claim 122 wherein the distal retainer comprises polypropylene or ceramic.

140. The system of claim 122 comprising fluid flow conduits, wherein the fluid flow conduits are associated with the distal retainer.

141. The system of claim 122 comprising:
axially-running fluid flow conduits on or in the inner surface of the housing; and
a solid insert,
wherein the axially-running fluid flow conduits and the solid insert form the distal retainer.

142. The system of claim 140 wherein the length of each conduit is less than the diameter of the conduit and wherein the length of the conduit is between 10% and 50% of the size of distal retainer.

143. The system of claim 141 wherein the length of each conduit is less than the diameter of the conduit and wherein the length of the conduit is between 10% and 50% of the size of distal retainer.

144. The system of claim 122 wherein:
the housing is smooth, or
the housing or the surface of the fluid sample is chemically treated.

145. The system of claim 144 wherein the smooth housing comprises a surface of 1.4 micro-inches RMS or less.

146. The system of claim 144 wherein the housing or the surface of the fluid sample is chemically treated with a negatively charged poly-electrolyte.

147. The system of claim 122 wherein the molecule of interest is a biomolecule.

148. The system of claim 147 wherein the biomolecule is a nucleic acid.

149. A method for isolating a molecule of interest in a fluid sample comprising the steps of:
a. providing a fluid sample purification system, wherein the system comprises:
a housing having a distal end with a distal opening adapted for the passage of a fluid and a proximal end with a proximal opening adapted for passage of a fluid;
adsorption material comprising a plurality of functionalized particles; and
a distal retainer and proximal retainer for retaining the adsorption material;
b. drawing a fluid sample into the fluid sample purification system;
c. contacting the plurality of functionalized particles with the fluid sample; and
d. aspirating and dispensing the fluid sample using an aspirating/dispensing cycle and fluid flow rate that maintains a consistent and consistently high rate of fluid flow, thereby expelling the fluid sample from the fluid sample purification system.

150. The method of claim 149 wherein the step of aspirating and dispensing comprises over-aspirating or over-dispensing the fluid sample.

151. The method of claim 149 wherein the step of aspirating and dispensing comprises two or more aspirating/dispensing cycles.
